(19) United States
(12) Patent Application Publication
(10) Pub. No.: US 2026/0247792 A1
(43) Pub. Date: Aug. 20, 2026

(54) ORGANIC COMPOUND, ELECTRONIC ELEMENT AND ELECTRONIC APPARATUS USING THE SAME

(71) Applicant: SHAANXI LIGHTE OPTOELECTRONICS MATERIAL CO., LTD., Xi'an, Shaanxi (CN)

(72) Inventors: Wenqiang LIU, Xi'an, Shaanxi (CN); Zhiyan JIA, Xi'an, Shaanxi (CN)

(21) Appl. No.: 19/161,769

(22) PCT Filed: Mar. 18, 2024
(86) PCT No.: PCT/CN2024/082251

§ 371 Date: Sep. 3, 2025

(30) Foreign Application Priority Data
Jun. 12, 2023 (CN) 202310693446.6

(51) Int. Cl. H10K 50/15; C07B 59/00; C09K 11/06; H10K 85/30; H10K 85/60

(52) U.S. Cl. CPC H10K 50/156; C07B 59/002; C09K 11/06; H10K 85/342; H10K 85/351; H10K 85/381; H10K 85/626; H10K 85/633; H10K 85/636; H10K 85/654; H10K 85/6572; H10K 85/6574; H10K 85/6576; C07B 2200/05; C09K 2211/1088

(57) ABSTRACT

The present application relates to an organic compound, an electronic element and electronic apparatus using the same. The organic compounds have structures as shown in Formula I, and applying the organic compounds to organic electroluminescent devices can significantly improve the performance of the devices.

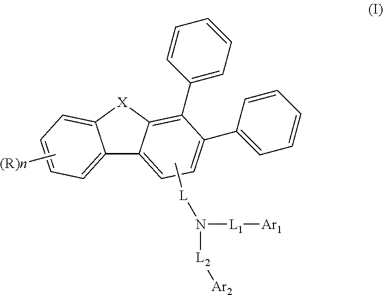

### CROSS-REFERENCE TO RELATED APPLICATIONS

The present application claims priority to Chinese Patent Application No. 202310693446.6 filed on Jun. 12, 2023, and the entire contents of the Chinese Patent Application are hereby incorporated in the present application.

### TECHNICAL FIELD

The present application relates to the field of organic electroluminescent technology, and in particular to an organic compound, electronic element and electronic apparatus using the same.

### BACKGROUND

With the development of electronic technology and the progress of material science, electronic elements and devices used to realize electroluminescence or photoelectric conversion have an increasingly wide range of applications. This kind of electronic element and device typically includes a cathode and an anode disposed opposite each other, and a functional layer disposed between the cathode and the anode. The functional layer is composed of multiple organic or inorganic film layers, and generally includes an energy conversion layer, a hole transport layer arranged between the energy conversion layer and the anode, and an electron transport layer arrange between the energy conversion layer and the cathode.

An organic electroluminescent device, for example, generally includes an anode, a hole transport layer, an electroluminescent layer as an energy conversion layer, an electron transport layer, and a cathode, which are sequentially stacked. When a voltage is applied to the cathode and the anode, an electric field is generated between the two electrodes, under the action of the electric field, electrons on the cathode side move towards the electroluminescent layer, and holes on the anode side also move towards the electroluminescent layer, electrons and holes combine in the electroluminescent layer to form excitons, which are in an excited state and release energy outward, thereby causing the electroluminescent layer to emit light externally.

However, the performances such as the efficiency and lifetime of the organic electroluminescent device still have room for further improvement compared to the product application requirements. Therefore, there is a necessity for the continued development of novel materials to further improve the performance of organic electroluminescent devices.

### SUMMARY

An object of the present application is to provide an organic compound and an electronic element, an electronic apparatus using the same, and the organic compound, when used in an organic electroluminescent device, can improve the performance of the device.

A first aspect of the present application provides an organic compound having a structure shown by a Formula I:

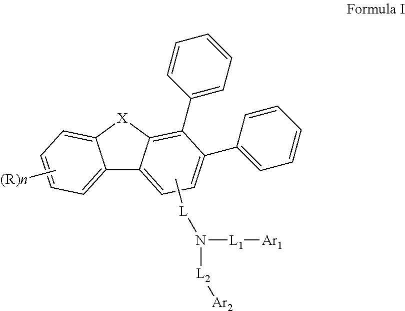

wherein, X is selected from O, S, or N(R₃);
R₃ is selected from an alkyl having 1 to 5 carbon atoms, a deuterated alkyl having 1 to 5 carbon atoms, a haloalkyl having 1 to 5 carbon atoms, an aryl having 6 to 20 carbon atoms, a deuterated aryl having 6 to 20 carbon atoms, or a heteroaryl having 3 to 20 carbon atoms;
L, L₁, and L₂ are the same or different, and are each independently selected from a single bond, a substituted or unsubstituted arylene having 6 to 30 carbon atoms, or a substituted or unsubstituted heteroarylene having 3 to 30 carbon atoms;
Ar₁ and Ar₂ are the same or different, and are each independently selected from a substituted or unsubstituted aryl having 6 to 30 carbon atoms, or a substituted or unsubstituted heteroaryl having 3 to 30 carbon atoms;
the substituent(s) of L, L₁, L₂, Ar₁, and Ar₂ are the same or different, and are each independently selected from a deuterium, a halogen, a cyano, an alkyl having 1 to 10 carbon atoms, a deuterated alkyl having 1 to 10 carbon atoms, a trialkylsilyl having 3 to 12 carbon atoms, a haloalkyl having 1 to 10 carbon atoms, an aryl having 6 to 20 carbon atoms, or a heteroaryl having 3 to 20 carbon atoms;
each R is independently selected from a deuterium, a halogen, a cyano, an alkyl having 1 to 10 carbon atoms, a haloalkyl having 1 to 10 carbon atoms, a deuterated alkyl having 1 to 10 carbon atoms, a trialkylsilyl having 3 to 12 carbon atoms, an aryl having 6 to 20 carbon atoms, or a heteroaryl having 3 to 20 carbon atoms;
n represents the number of R and n is selected from 0, 1, 2, 3 or 4; when n is greater than 1, any two Rs are the same or different.

A second aspect of the present application provides an electronic element, comprising an anode and a cathode disposed opposite each other, and a functional layer disposed between the anode and the cathode; wherein the functional layer comprises the organic compounds described above.

A third aspect of the present application provides an electronic apparatus, comprising the electronic element described in the second aspect.

The main structural feature of the organic compounds of the present application is a dibenzo five-membered heterocyclic ring

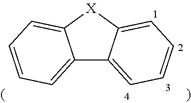

in which three substitutions are on the same benzene ring, wherein the substituents in positions 1 and 2 of the dibenzo five-membered heterocyclic ring are both phenyl groups, and either position 3 or 4 is connected to an arylamine group. In such a connection mode, on the one hand, the phenyl attached to positions 1 and 2 of the dibenzo five-membered heterocyclic ring can enhance the spatial stericity of the molecule and improve the carrier mobility of the compound; on the other hand, the arylamine attached to position 3 or 4 of the dibenzo five-membered heterocyclic ring increases the degree of molecular conjugation, which effectively improves the transport efficiency of the compound and makes the compound have a high Tg, and therefore prevents the compound from crystallizing when driven in the OLED device, and thus prolongs the lifetime of the device. When the compounds of the present application are used as hole transport materials, they can improve the carrier balance in the organic light-emitting layer, broaden the carrier recombination zone, enhance the efficiency of exciton generation and utilization, and improve the efficiency and lifetime of the device.

Other features and advantages of the present application will be described in detail in the subsequent detailed description.

### BRIEF DESCRIPTION OF THE DRAWINGS

The drawings are used for a further understanding of the present application and constitute a part of the specification and are used to explain the present application together with the following specific embodiments, but do not constitute a limitation of the present application.

FIG. 1 is a schematic structural diagram of an organic electroluminescent device according to one embodiment of the present application.

FIG. 2 is a schematic diagram of the first electronic apparatus according to one embodiment of the present application.

FIG. 3 is a schematic structural diagram of a photoelectric conversion device according to one embodiment of the present application.

FIG. 4 is a schematic diagram of the second electronic apparatus according to one embodiment of the present application.

### REFERENCE SIGNS

100, Anode; 200, Cathode; 300, Functional layer; 310, Hole injection layer; 320, Hole transport layer, 321, First hole transport layer; 322, Second hole transport layer; 330, Organic light-emitting layer; 340, Electron transport layer; 350, Electron injection layer; 360, Photoelectric conversion layer; 400, First electronic apparatus: 500, Second electronic apparatus.

### DETAILED DESCRIPTION OF THE EMBODIMENTS

Exemplary embodiments will now be described more comprehensively with reference to the accompanying drawings. The exemplary embodiments, however, can be implemented in a variety of forms and should not be interpreted as being limited to the examples set forth herein; On the contrary, these examples are provided to make the present application more comprehensive and complete, and to convey the concepts of these exemplary embodiments fully to those skill in the art. Features, structures, or characteristics described herein can be combined in one or more embodiment(s) in any suitable manner. In the following description, many specific details are provided to give a full understanding of the examples of the present application.

In a first aspect, the present application provides an organic compound having a structure shown by a Formula I below:

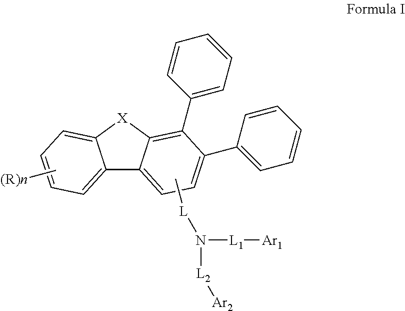

wherein, X is selected from O, S, or N(R₃);
R₃ is selected from an alkyl having 1 to 5 carbon atoms, a deuterated alkyl having 1 to 5 carbon atoms, a haloalkyl having 1 to 5 carbon atoms, an aryl having 6 to 20 carbon atoms, a deuterated aryl having 6 to 20 carbon atoms, or a heteroaryl having 3 to 20 carbon atoms;
L, L₁, and L₂ are the same or different, and are each independently selected from a single bond, a substituted or unsubstituted arylene having 6 to 30 carbon atoms, or a substituted or unsubstituted heteroarylene having 3 to 30 carbon atoms;
Ar₁ and Ar₂ are the same or different, and are each independently selected from a substituted or unsubstituted aryl having 6 to 30 carbon atoms, or a substituted or unsubstituted heteroaryl having 3 to 30 carbon atoms;
the substituent(s) of L, L₁, L₂, Ar₁, and Ar₂ are the same or different, and are each independently selected from a deuterium, a halogen, a cyano, an alkyl having 1 to 10 carbon atoms, a deuterated alkyl having 1 to 10 carbon atoms, a trialkylsilyl having 3 to 12 carbon atoms, a haloalkyl having 1 to 10 carbon atoms, an aryl having 6 to 20 carbon atoms, or a heteroaryl having 3 to 20 carbon atoms;
each R is independently selected from a deuterium, a halogen, a cyano, an alkyl having 1 to 10 carbon atoms, a haloalkyl having 1 to 10 carbon atoms, a deuterated alkyl having 1 to 10 carbon atoms, a trialkylsilyl having 3 to 12 carbon atoms, an aryl having 6 to 20 carbon atoms, or a heteroaryl having 3 to 20 carbon atoms;
n represents the number of R and n is selected from 0, 1, 2, 3 or 4; when n is greater than 1, any two Rs are the same or different.

In the present application, the terms “optional” and “optionally” mean that the event or circumstance described later may, but not necessarily, occur, and the description includes the circumstances under which the event or environment occurs or does not occur. For example, “optionally, two adjacent substituents form a ring” means that these two substituents may, but not necessarily, form a ring, including scenarios both where two adjacent substituents form a ring and where two adjacent substituents do not form a ring.

In the present application, the descriptive expressions “be each independently selected from” and “be respectively and independently selected from” may be interchanged and all these expressions should be interpreted in a broad sense. They may both refer to specific options expressed by the same symbol in separate groups are mutually non-influential, and to specific options expressed by the same symbols within the same group are mutually non-influential. For example,

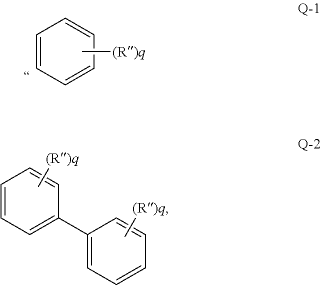

wherein each q is independently 0, 1, 2, or 3, and each R″ is independently selected from the group consisting of hydrogen, deuterium, fluorine, and chlorine” means that Formula Q-1 represents that there are q substituent R(s)″ on the benzene ring, and each R″ may be the same or different, with mutual non-influence between the options for each R″; Formula Q-2 represents that there are q substituent R(s)″ on each benzene ring of biphenyl, and the number q of R″ substituent(s) on the two benzene rings may be the same or different, and each R″ may be the same or different, with mutual non-influence between the options for each R″.

In the present application, the term “substituted or unsubstituted” means that the functional group defined by such term may or may not have a substituent (hereinafter referred to as Rc for ease of description). For example, “substituted or unsubstituted aryl” refers to aryl having a substituent Rc or unsubstituted aryl. Among them, the above substituent, i.e., Rc, may be, for example, deuterium, halogen, cyano, alkyl, trialkylsilyl, haloalkyl, deuterated alkyl, cycloalkyl, aryl, heteroaryl, etc.

In the present application, the number of carbon atoms of a substituted or unsubstituted functional group refers to the total number of carbon atoms. For example, if L₁ is substituted with an arylene having 12 carbon atoms, the total number of carbon atoms in the arylene and its substituents is 12.

In this application, substituent “D” represents deuterium.

In the present application, an aryl refers to an optional functional group or a substituent derived from an aromatic carbon ring. Aryl may be a monocyclic aryl (e.g., phenyl) or a polycyclic aryl. In other words, aryl may be a monocyclic aryl, a fused-ring aryl, two or more monocyclic aryls linked by carbon-carbon bond, a monocyclic aryl and a fused-ring aryl linked by carbon-carbon bond, or two or more fused-ring aryls linked by carbon-carbon bond. That is, unless otherwise specified, two or more aromatic groups linked by carbon-carbon bond may also be regarded as an aryl in the present application. Among them, a fused-ring aryl may include, for example, a bicyclic fused aryl (e.g., naphthyl), a tricyclic fused aryl (e.g., phenanthryl, fluorenyl, and anthryl), etc. The aryl does not contain heteroatoms such as B, N, O, S, P, Se and Si. For example, in the present application, biphenyl, terphenyl, and the like are aryl groups. Examples of aryl include, but are not limited to, phenyl, naphthyl, fluorenyl, anthracenyl, phenanthryl, biphenyl, terphenyl, benzo[9,10]phenanthryl, pyrenyl, benzofluoranthryl, chrysenyl, and the like. In the present application, “arylene” involved refers to a divalent group formed by further removing one hydrogen atom from aryl.

In the present application, substituted aryl may mean that one or more than two hydrogen atom(s) in the aryl are replaced by a group such as deuterium atom, halogen, cyano, aryl, heteroaryl, trialkylsilyl, alkyl, cycloalkyl, haloalkyl, deuterated alkyl etc. It should be understood that the number of carbon atoms in a substituted aryl refers to the total number of carbon atoms of an aryl and the substituents on the aryl. For example, substituted aryl having 18 carbon atoms, refers to the total number of carbon atoms of the aryl and the substituents thereon is 18.

In the present application, “heteroaryl” refers to a monovalent aromatic ring containing at least one heteroatom or a derivative thereof. The heteroatoms may be one or more of B, O, N, P, Si, Se, and S. A heteroaryl may be a monocyclic heteroaryl or a polycyclic heteroaryl. In other words, a heteroaryl may be a single aromatic ring system, or multiple aromatic ring systems connected through conjugated carbon-carbon bonds, with any of the aromatic ring systems being an aromatic monocyclic ring or an aromatic fused ring. Exemplarily, heteroaryl may include thienyl, furyl, pyrrolyl, imidazolyl, thiazolyl, oxazolyl, oxadiazolyl, triazolyl, pyridyl, dipyridyl, pyrimidinyl, triazinyl, acridinyl, pyridazinyl, pyrazinyl, quinolinyl, quinazolinyl, quinoxalinyl, phenoxazinyl, phthalazinyl, pyridopyrimidinyl, pyridopyrazinyl, pyrazinopyrazinyl, isoquinolinyl, indolyl, carbazolyl, benzoxazolyl, benzimidazolyl, benzothiazolyl, benzocarbazolyl, benzothienyl, dibenzothienyl, thienothienyl, benzofuranyl, phenanthrolinyl, isoxazolyl, thiadiazolyl, phenothiazinyl, silafluorenyl, dibenzofuranyl, as well as N-phenylcarbazolyl, N-pyridylcarbazolyl, N-methylcarbazolyl, etc., but not limited thereto. In the present application, “heteroarylene” involved refers to a divalent group formed by further removing one hydrogen atom from heteroaryl.

In the present application, substituted heteroaryl may mean that one or more than two hydrogen atom(s) in the heteroaryl are replaced by a group such as deuterium atom, halogen, cyano, aryl, heteroaryl, trialkylsilyl, alkyl, cycloalkyl, haloalkyl, deuterated alkyl etc. It should be understood that the number of carbon atoms in the substituted heteroaryl refers to the total number of carbon atoms in the heteroaryl and the substituents thereof.

In the present application, the number of carbon atoms of aryl as a substituent may be 6 to 20, for example, the number of carbon atoms may be 6, 7, 8, 9, 10, 11, 12, 13, 14, 15, 16, 17, 18, 19, or 20, and specific examples of aryl as a substituent include, but are not limited to, phenyl, biphenyl, naphthyl, anthracenyl, or chrysenyl.

In the present application, the number of carbon atoms of heteroaryl as a substituent may be 3 to 20, for example, the number of carbon atoms may be 3, 4, 5, 6, 7, 8, 9, 10, 11, 12, 13, 14, 15, 16, 17, 18, 19, or 20, and specific examples of heteroaryl as a substituent include, but are not limited to, pyridyl, pyrimidinyl, carbazolyl, dibenzofuranyl, dibenzothienyl, quinolinyl, quinazolinyl, quinoxalinyl, or isoquinolinyl.

In the present application, an alkyl having 1 to 10 carbon atoms may include a straight-chain alkyl having 1 to 10 carbon atoms, and a branched alkyl having 3 to 10 carbon atoms. The number of carbon atoms of alkyl may be, for example, 1, 2, 3, 4, 5, 6, 7, 8, 9, or 10, and the specific examples of alkyl include but are not limited to, methyl, ethyl, n-propyl, isopropyl, n-butyl, isobutyl, tert-butyl, n-pentyl, isopentyl, neopentyl, n-hexyl, n-heptyl, n-octyl, 2-ethylhexyl, nonyl, decyl, 3,7-dimethyloctyl, and the like.

In the present application, the halogen group may be, for example, fluorine, chlorine, bromine, or iodine.

In the present application, the specific examples of a trialkylsilyl include, but are not limited to, a trimethylsilyl, a triethylsilyl, etc.

In the present application, the specific examples of a haloalkyl include, but are not limited to, a trifluoromethyl.

In the present application, the specific examples of a deuterated alkyl include, but are not limited to, a trideuteromethyl.

In the present application, a non-positioned connection bond refers to a single bond

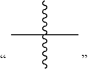

extending from a ring system, which represents that one end of the connection bond may connect to any position in the ring system through which the bond passes, and the other end connects to the rest of the compound molecule. For example, as shown in Formula (f) below, a naphthyl represented by Formula (f) is connected to other positions of the molecule through two non-positioned connection bonds passing through the bicyclic structure, which indicates any of possible connecting mode shown in Formulae (f-1) to (f-10):

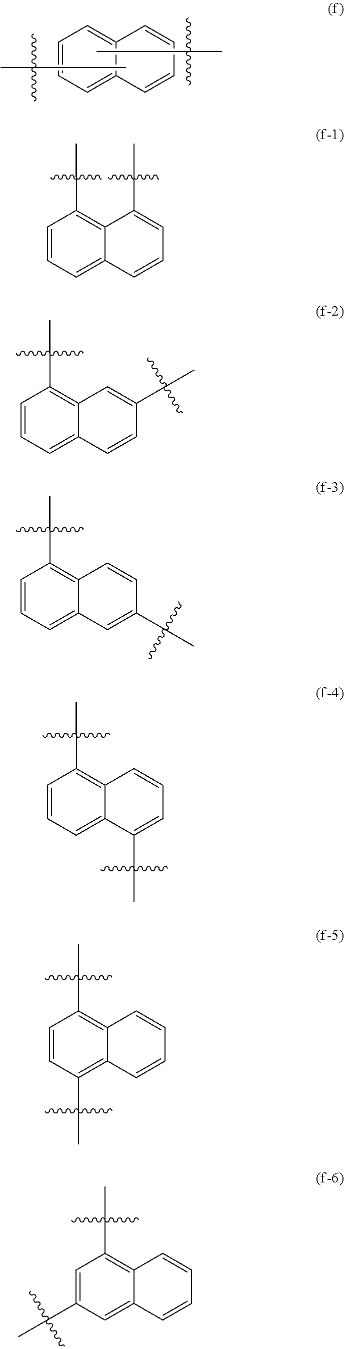

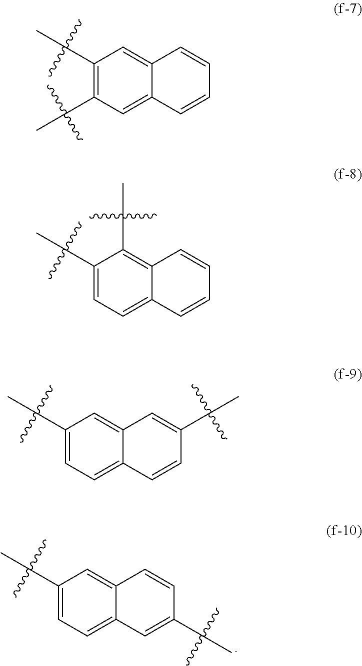 [SMILES: *c1cc2ccccc2cc1* ; *c1ccc2ccccc2c1* ; *c1ccc2ccc(*)cc2c1 ; *c1ccc2cc(*)ccc2c1]

As another example, as shown in Formula (X′) below, a dibenzofuranyl represented by Formula (X′) is connected to other positions of the molecule through a non-positioned connection bond extending from the center of benzene ring at one side, which indicates any of possible connecting mode shown in Formulae (X′-1) to (X′-4):

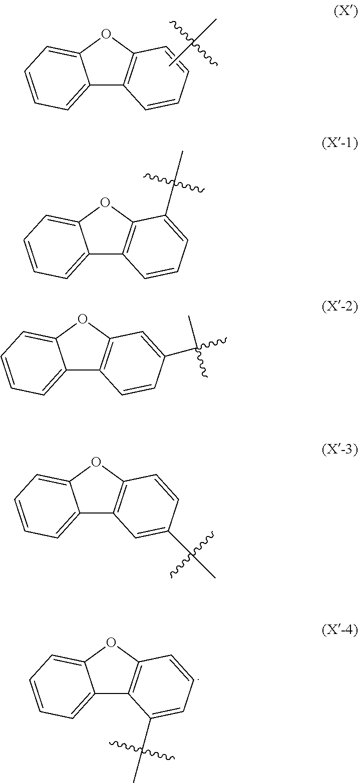

In some embodiments of the present application, the organic compound has the structure shown in Formula 1 to Formula 6 below:

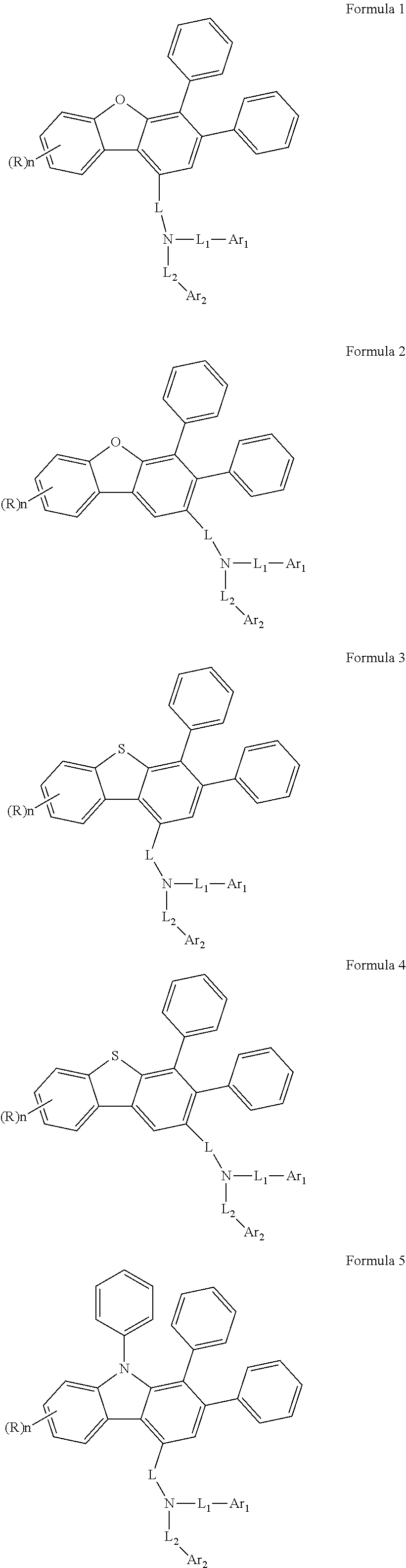

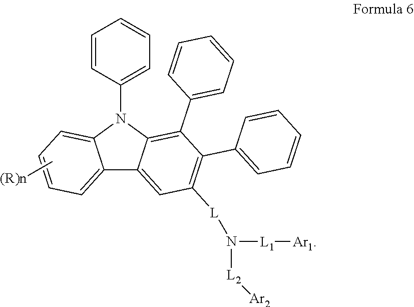

In some embodiments of the present application, R₃ is selected from a methyl, an ethyl, an isopropyl, a tert-butyl, a trideuteromethyl, a trifluoromethyl, a phenyl, a deuterophenyl, a naphthyl, a biphenyl, a dibenzofuranyl, a dibenzothienyl, or a carbazolyl.

In some embodiments of the present application, each R is independently selected from a deuterium, a fluorine, a cyano, a methyl, an ethyl, an isopropyl, a tert-butyl, a trifluoromethyl, a trimethylsilyl, a phenyl, a naphthyl, a biphenyl, a dibenzofuranyl, a dibenzothienyl, or a carbazolyl.

In some embodiments, L, L₁, and L₂ are each independently selected from a single bond, a substituted or unsubstituted arylene having 6 to 18 carbon atoms, or a substituted or unsubstituted heteroarylene having 12 to 18 carbon atoms. For example, L, L₁, and L₂ are each independently selected from a single bond, a substituted or unsubstituted arylene having 6, 7, 8, 9, 10, 11, 12, 13, 14, 15, 16, 17, or 18 carbon atoms, or a substituted or unsubstituted heteroarylene having 12, 13, 14, 15, 16, 17, or 18 carbon atoms.

Alternatively, the substituent(s) of L, L₁, and L₂ are each independently selected from a deuterium, a fluorine, a cyano, a trifluoromethyl, a trimethylsilyl, an alkyl having 1 to 5 carbon atoms, or a phenyl.

In some embodiments of the present application, L, L₁, and L₂ are each independently selected from a single bond, a substituted or unsubstituted phenylene, a substituted or unsubstituted naphthylene, a substituted or unsubstituted biphenylene, a substituted or unsubstituted dibenzofuranylene, a substituted or unsubstituted dibenzothienylene, or a substituted or unsubstituted carbazolylene.

Alternatively, the substituent(s) of L, L₁, and L₂ are each independently selected from a deuterium, a fluorine, a cyano, a trifluoromethyl, a trimethylsilyl, a methyl, an ethyl, an isopropyl, a tert-butyl, or a phenyl.

In some embodiments of the present application, L, L₁, and L₂ are each independently selected from a single bond or the following groups:

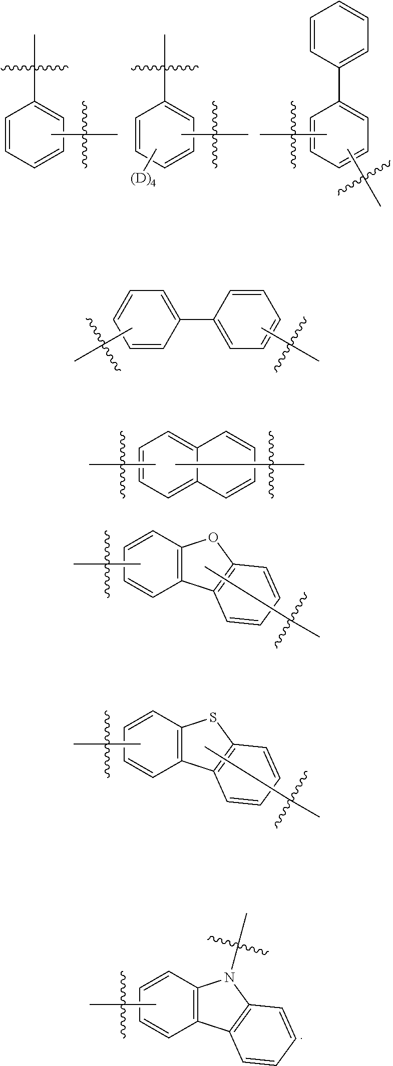

In some embodiments of the present application, L is selected from a single bond or the following groups:

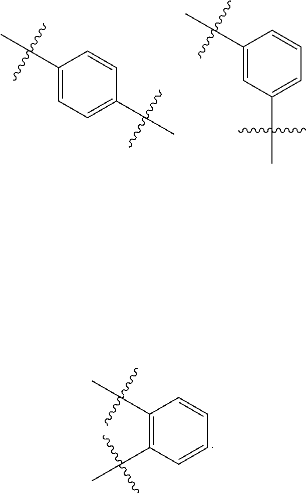 [SMILES: *c1ccc(*)cc1 ; *c1cccc(*)c1 ; *c1ccccc1* ; "."]

In some embodiments of the present application, L₁ and L₂ are each independently selected from a single bond or the following groups:

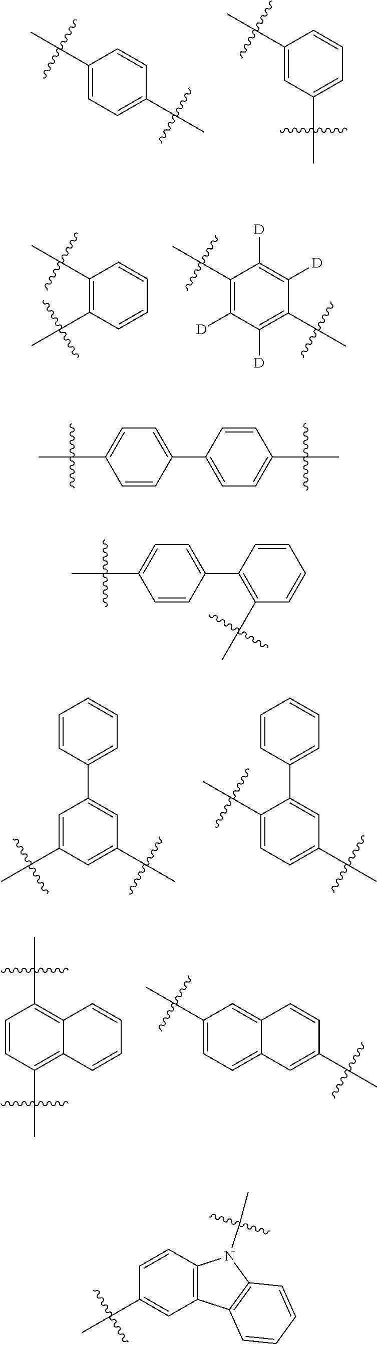

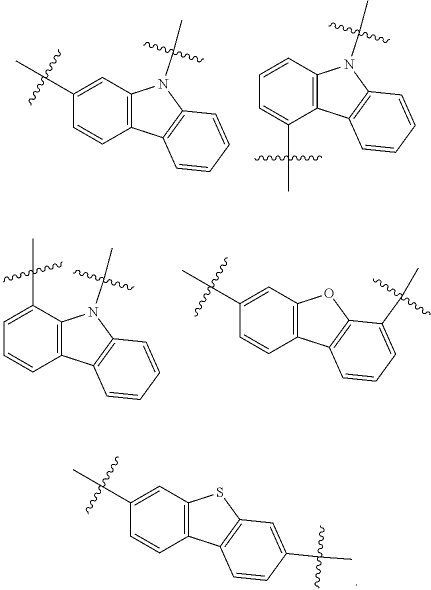

In some embodiments of the present application, Ar₁ and Ar₂ are each independently selected from a substituted or unsubstituted aryl having 6 to 25 carbon atoms, or a substituted or unsubstituted heteroaryl having 12 to 18 carbon atoms. For example, Ar₁ and Ar₂, are each independently selected from a substituted or unsubstituted aryl having 6, 7, 8, 9, 10, 11, 12, 13, 14, 15, 16, 17, 18, 10, 20, 21, 22, 23, 24, or 25 carbon atoms, or a substituted or unsubstituted heteroaryl having 12, 13, 14, 15, 16, 17, or 18 carbon atoms.

Alternatively, the substituent(s) of Ar₁ and Ar₂ are each independently selected from a deuterium, a fluorine, a cyano, a trialkylsilyl having 3 to 6 carbon atoms, a haloalkyl having 1 to 5 carbon atoms, a deuterated alkyl having 1 to 5 carbon atoms, an alkyl having 1 to 5 carbon atoms, an aryl having 6 to 12 carbon atoms, or a heteroaryl having 5 to 12 carbon atoms.

In some embodiments of the present application, Ar₁ and Ar₂ are each independently selected from a substituted or unsubstituted phenyl, a substituted or unsubstituted naphthyl, a substituted or unsubstituted biphenyl, a substituted or unsubstituted terphenyl, a substituted or unsubstituted fluorenyl, a substituted or unsubstituted 9,9-spirobifluorenyl, a substituted or unsubstituted triphenylene, a substituted or unsubstituted phenanthryl, a substituted or unsubstituted cyclopentanespirofluorene, a substituted or unsubstituted cyclohexanespirofluorene, a substituted or unsubstituted dibenzofuranyl, a substituted or unsubstituted dibenzothienyl, or a substituted or unsubstituted carbazolyl.

Alternatively, the substituent(s) of Ar₁ and Ar₂ are each independently selected from a deuterium, a fluorine, a cyano, a trimethylsilyl, a trifluoromethyl, a methyl, an ethyl, an isopropyl, a tert-butyl, a trideuteromethyl, a phenyl, a naphthyl, a biphenyl, a dibenzofuranyl, a dibenzothienyl, or a carbazolyl.

In some embodiments of the present application, Ar₁ and Ar₂ are each independently selected from the group consisting of the following groups:

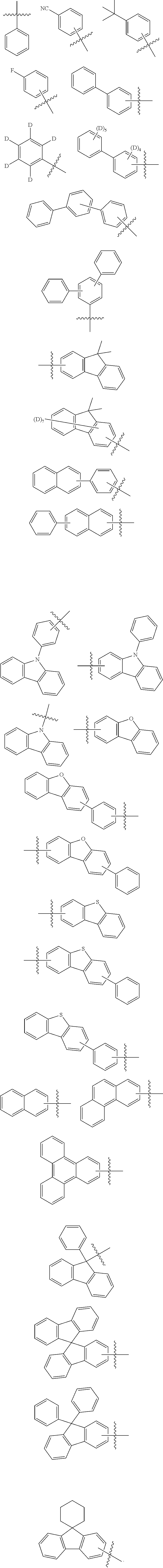

Further alternatively, Ar₁ and Ar₂ are each independently selected from the group consisting of the following groups:

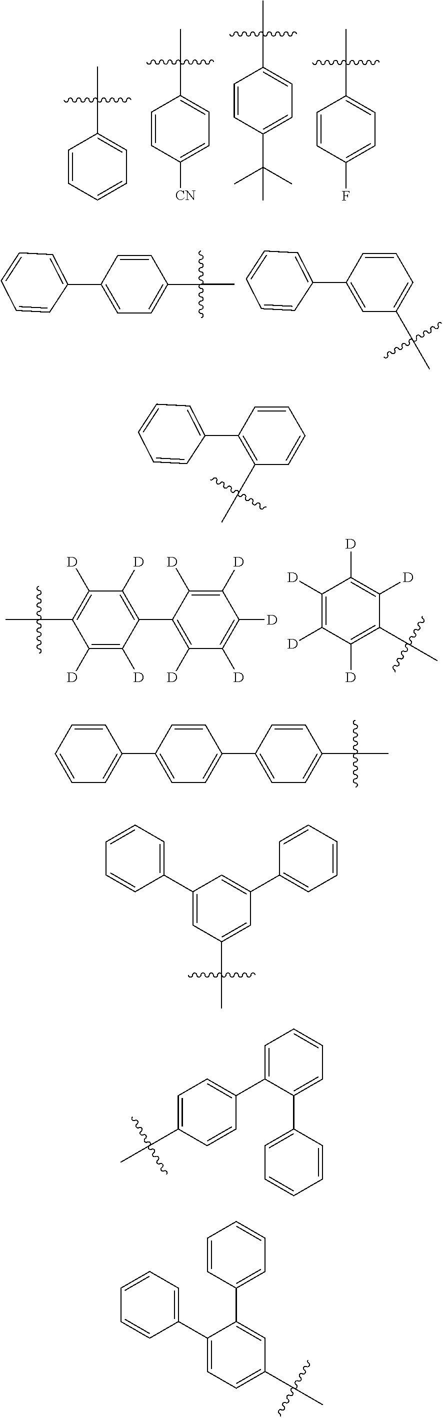

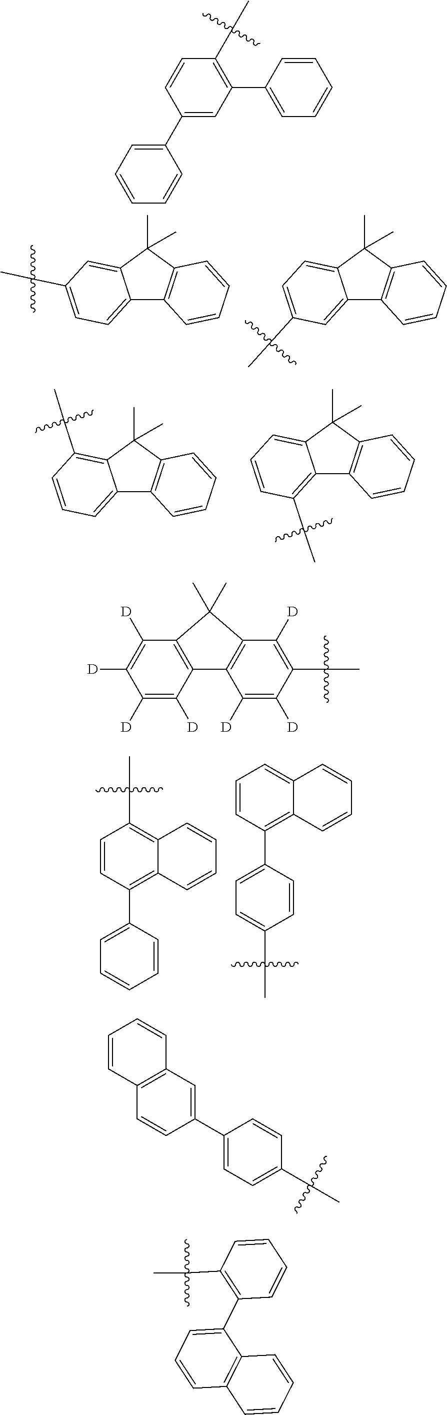

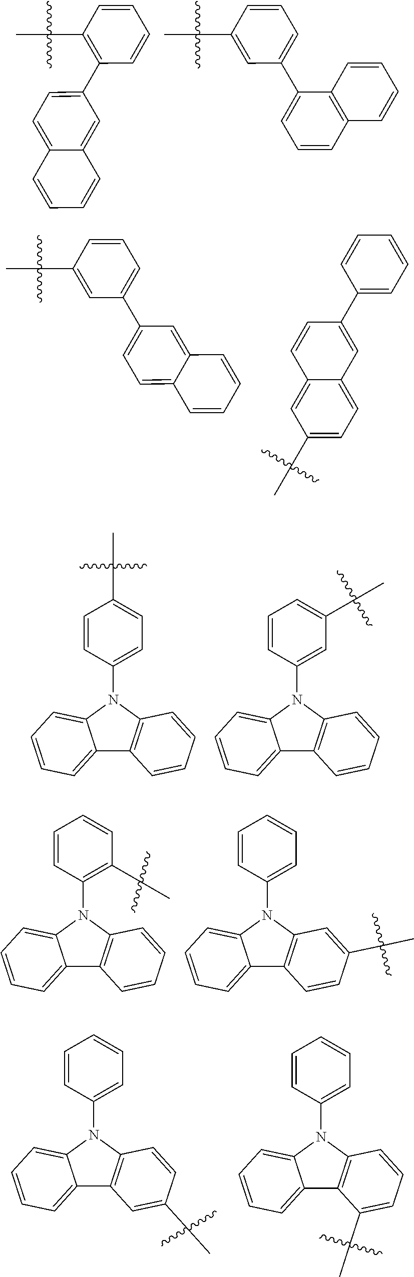

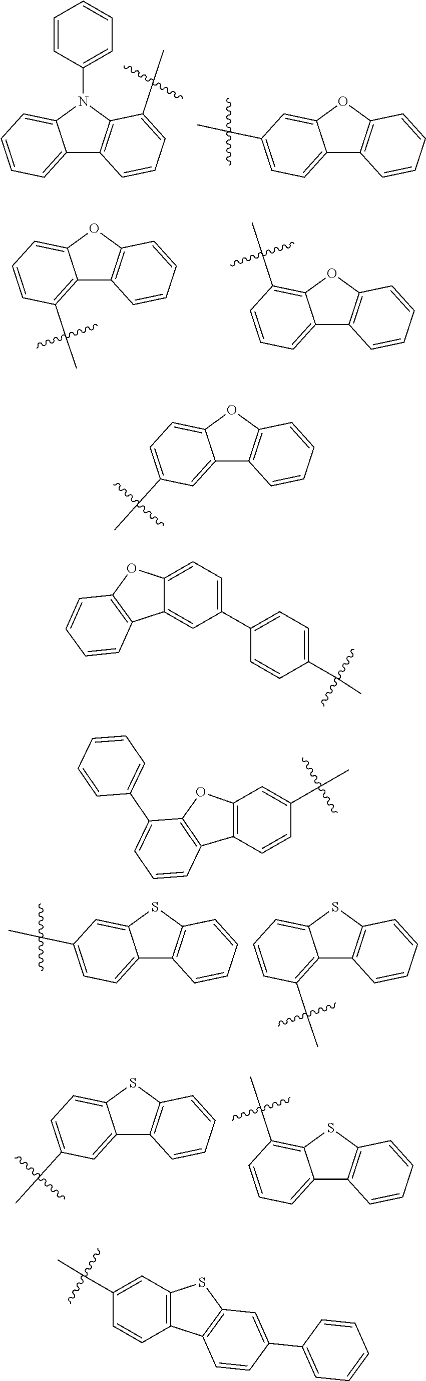

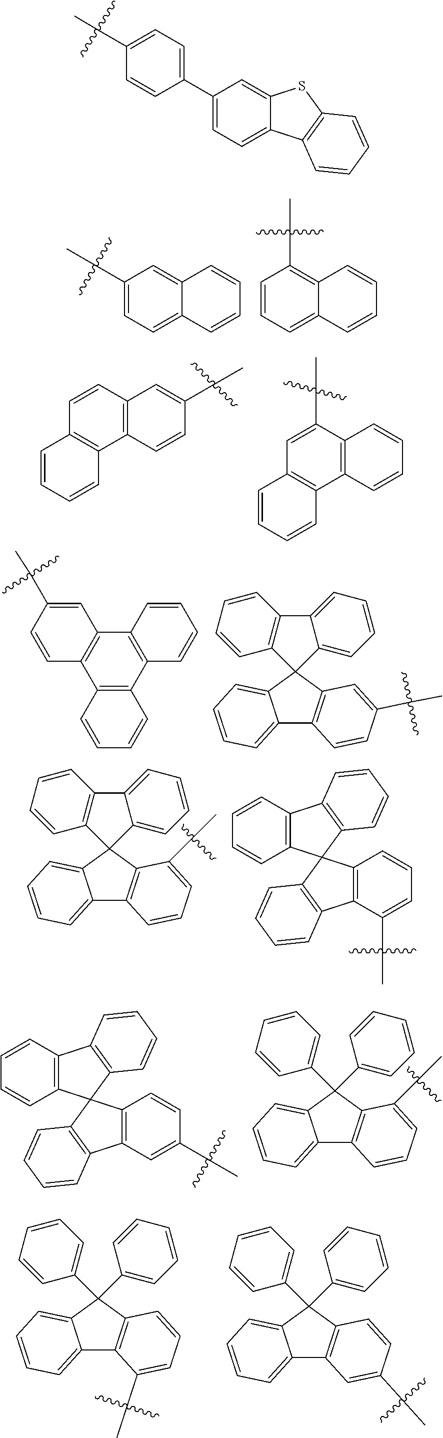

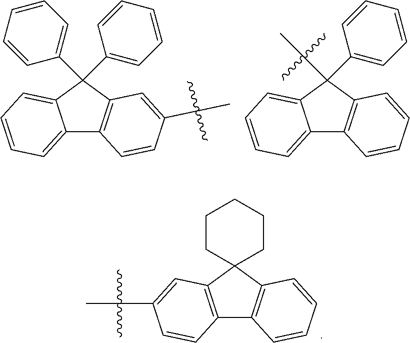 [SMILES: *c1ccc2c(c1)C(c1ccccc1)(c1ccccc1)c1ccccc1-2 ; *C1(c2ccccc2)c2ccccc2-c2ccccc21 ; *c1ccc2c(c1)C1(CCCCC1)c1ccccc1-2]

In some embodiments of the present application,

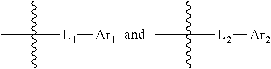

are each independently selected from the group consisting of the following groups:

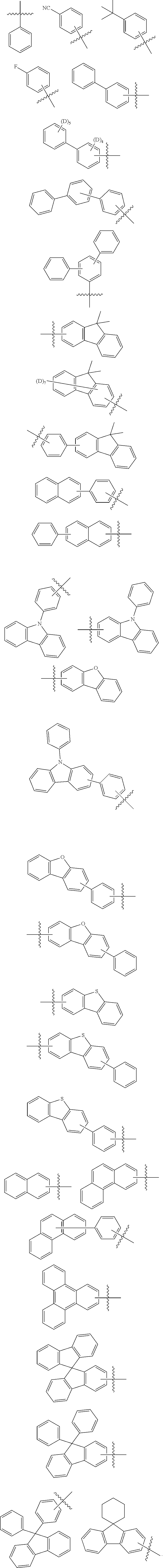

In some embodiments of the present application,

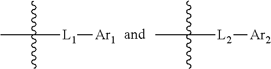

are each independently selected from the group consisting of the following groups:

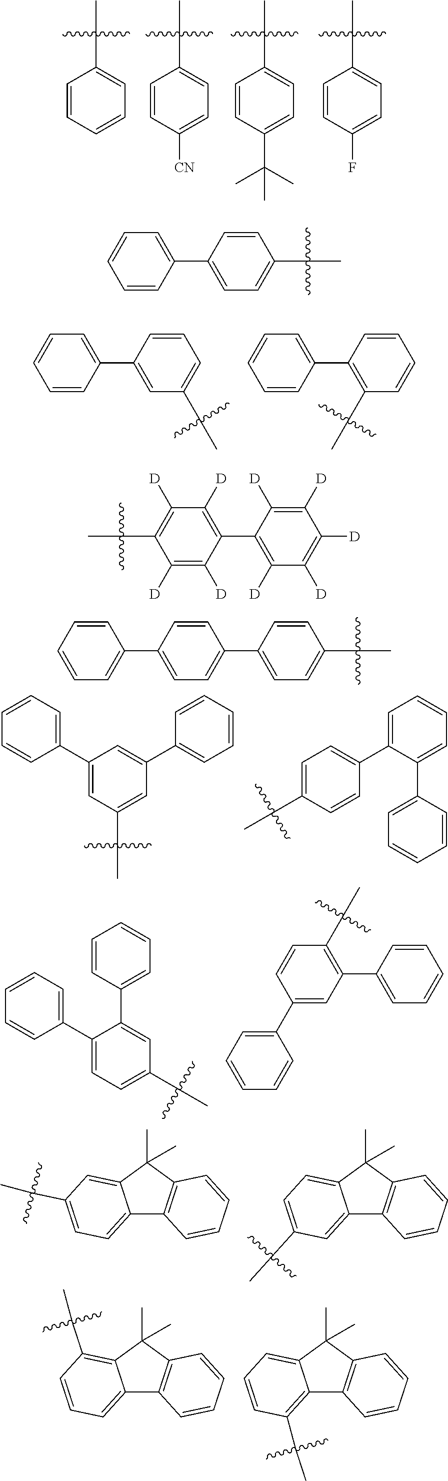

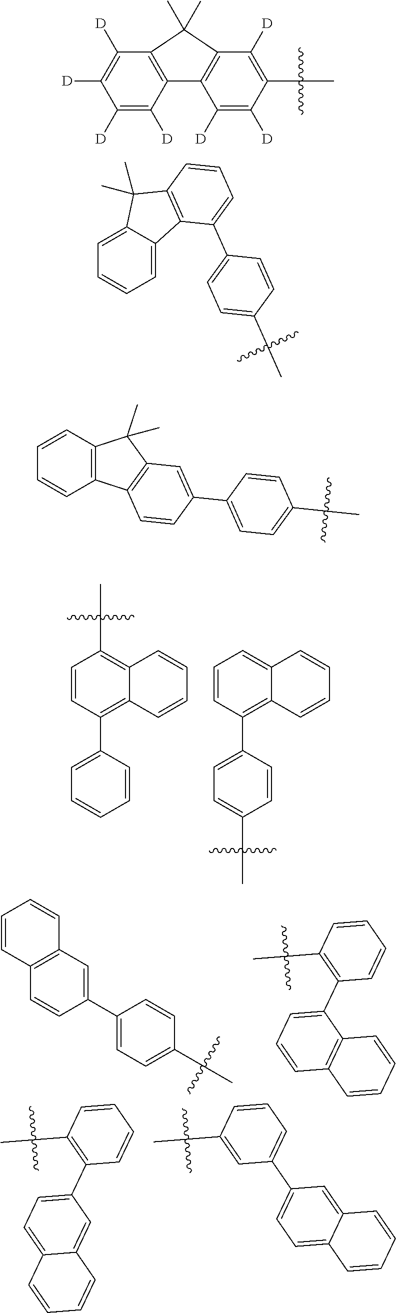

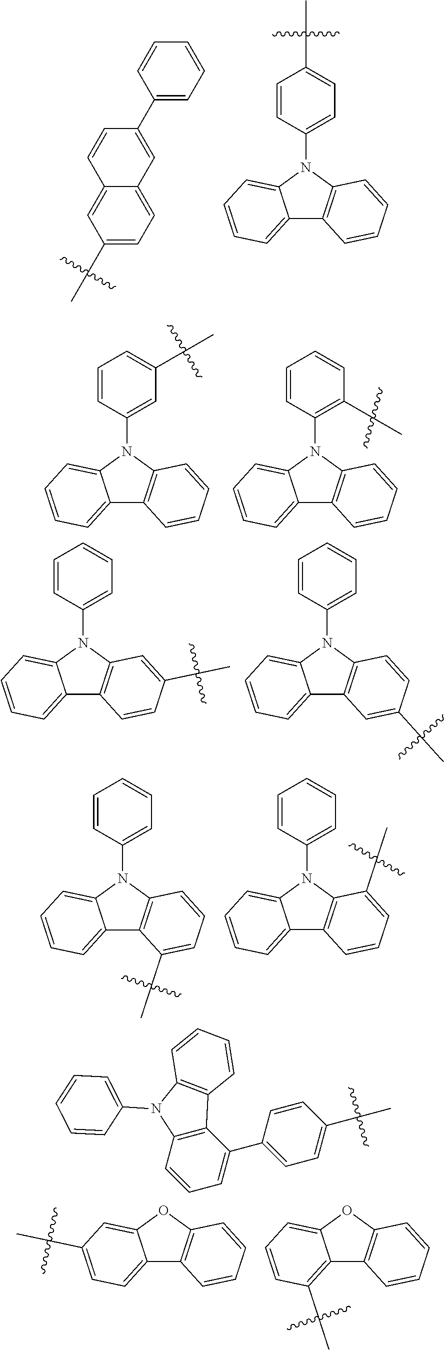

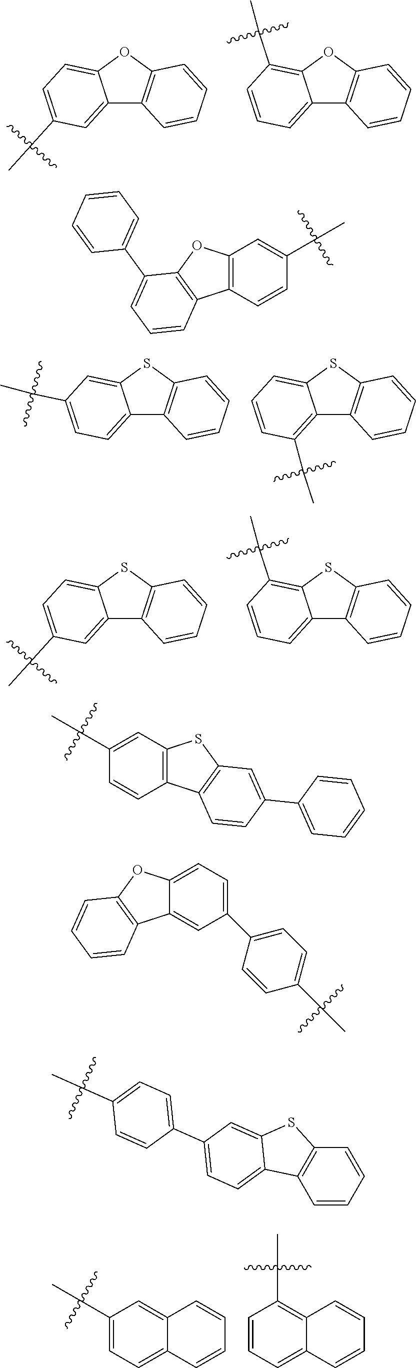

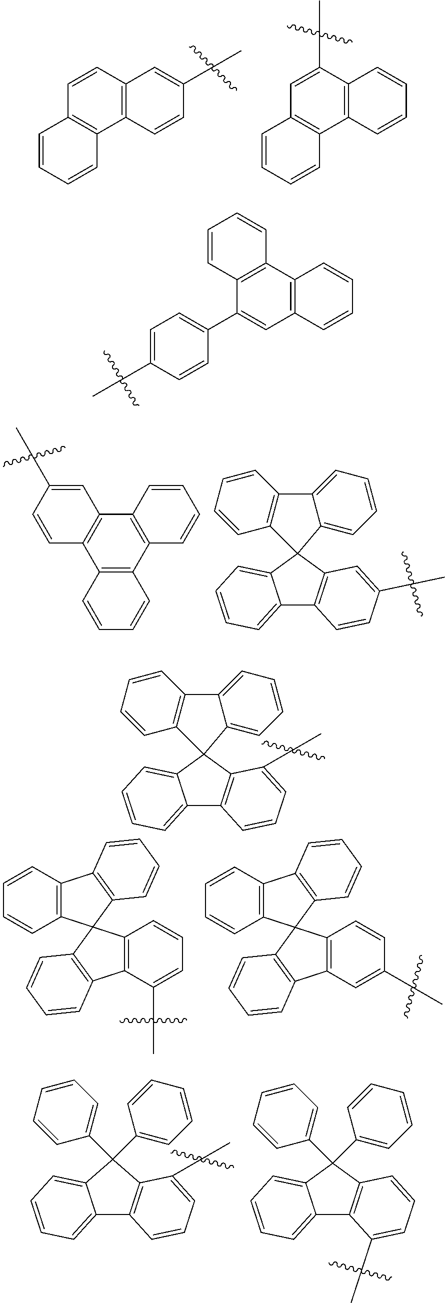

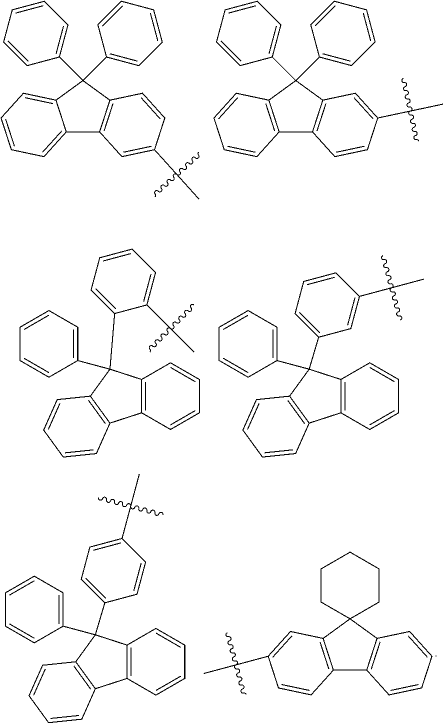

Specifically, the organic compound is selected from the group consisting of the following compounds:

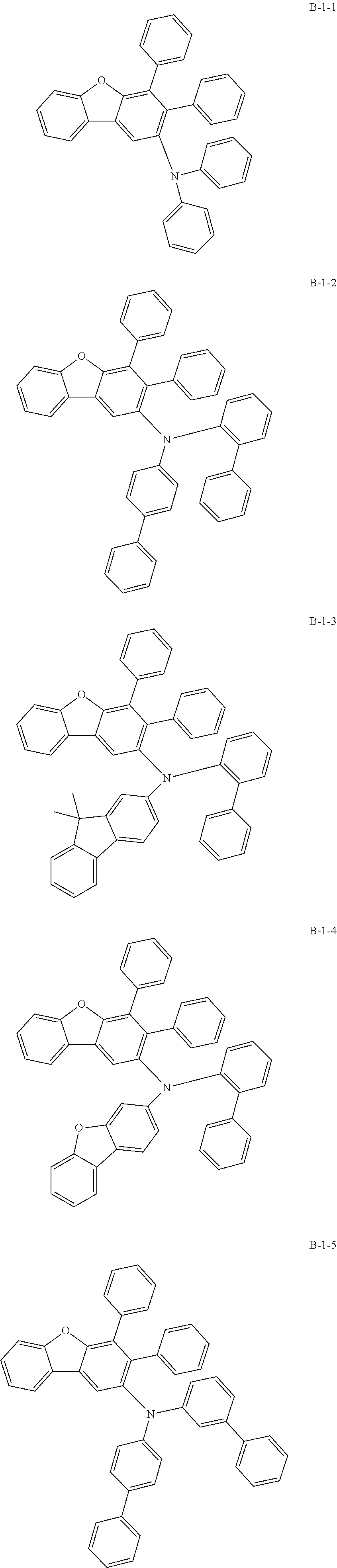 [SMILES: c1ccc(-c2c(N(c3ccccc3)c3ccccc3)cc3c(oc4ccccc43)c2-c2ccccc2)cc1 ; c1ccc(-c2ccc(N(c3ccccc3-c3ccccc3)c3cc4c(oc5ccccc54)c(-c4ccccc4)c3-c3ccccc3)cc2)cc1 ; CC1(C)c2ccccc2-c2ccc(N(c3ccccc3-c3ccccc3)c3cc4c(oc5ccccc54)c(-c4ccccc4)c3-c3ccccc3)cc21 ; c1ccc(-c2ccccc2N(c2ccc3c(c2)oc2ccccc23)c2cc3c(oc4ccccc43)c(-c3ccccc3)c2-c2ccccc2)cc1 ; c1ccc(-c2ccc(N(c3cccc(-c4ccccc4)c3)c3cc4c(oc5ccccc54)c(-c4ccccc4)c3-c3ccccc3)cc2)cc1]

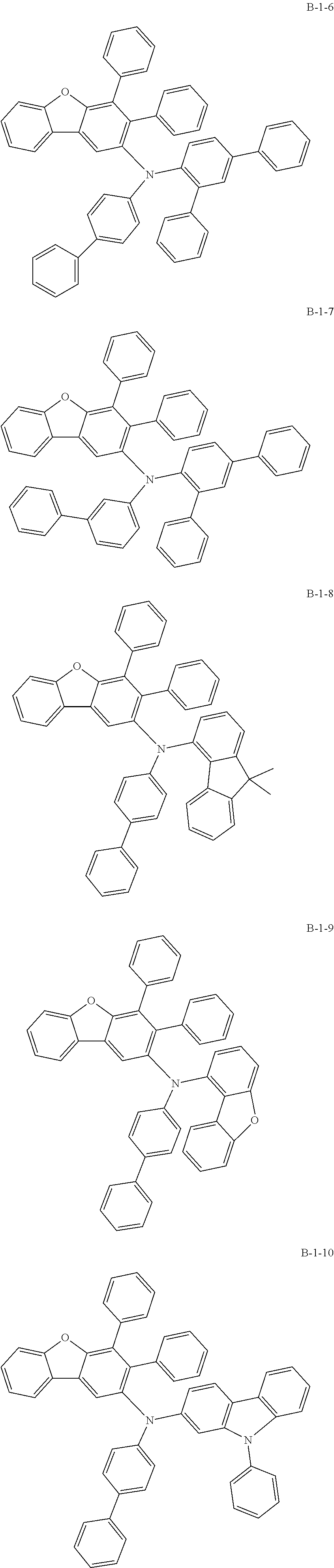 [SMILES: c1ccc(-c2ccc(N(c3ccc(-c4ccccc4)cc3-c3ccccc3)c3cc4c(oc5ccccc54)c(-c4ccccc4)c3-c3ccccc3)cc2)cc1 ; c1ccc(-c2cccc(N(c3ccc(-c4ccccc4)cc3-c3ccccc3)c3cc4c(oc5ccccc54)c(-c4ccccc4)c3-c3ccccc3)c2)cc1 ; CC1(C)c2ccccc2-c2c(N(c3ccc(-c4ccccc4)cc3)c3cc4c(oc5ccccc54)c(-c4ccccc4)c3-c3ccccc3)cccc21 ; c1ccc(-c2ccc(N(c3cc4c(oc5ccccc54)c(-c4ccccc4)c3-c3ccccc3)c3cccc4oc5ccccc5c34)cc2)cc1 ; c1ccc(-c2ccc(N(c3ccc4c5ccccc5n(-c5ccccc5)c4c3)c3cc4c(oc5ccccc54)c(-c4ccccc4)c3-c3ccccc3)cc2)cc1]

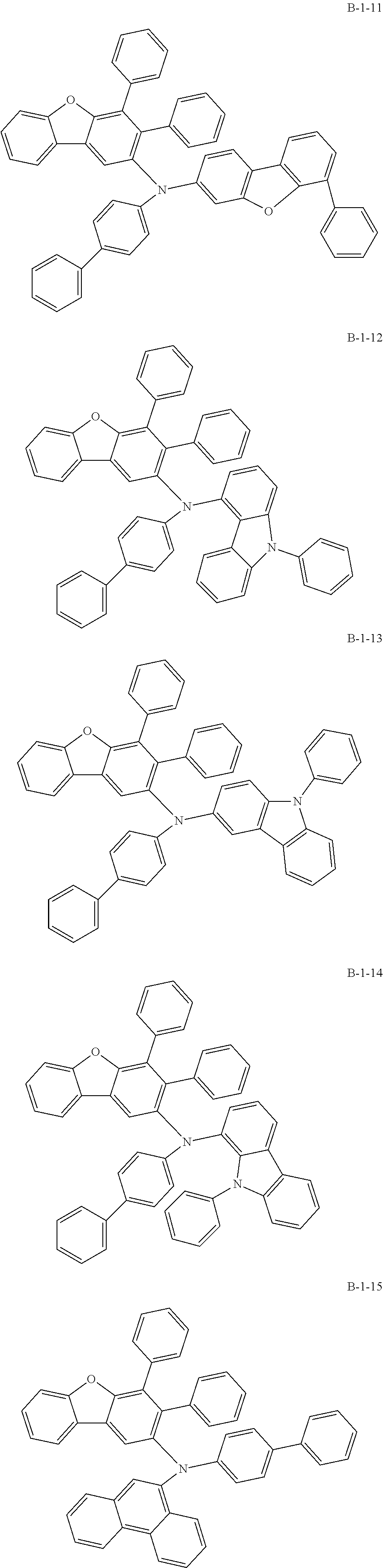 [SMILES: c1ccc(-c2ccc(N(c3ccc4c(c3)oc3c(-c5ccccc5)cccc34)c3cc4c(oc5ccccc54)c(-c4ccccc4)c3-c3ccccc3)cc2)cc1 ; c1ccc(-c2ccc(N(c3cc4c(oc5ccccc54)c(-c4ccccc4)c3-c3ccccc3)c3cccc4c3c3ccccc3n4-c3ccccc3)cc2)cc1 ; c1ccc(-c2ccc(N(c3ccc4c(c3)c3ccccc3n4-c3ccccc3)c3cc4c(oc5ccccc54)c(-c4ccccc4)c3-c3ccccc3)cc2)cc1 ; c1ccc(-c2ccc(N(c3cc4c(oc5ccccc54)c(-c4ccccc4)c3-c3ccccc3)c3cccc4c5ccccc5n(-c5ccccc5)c34)cc2)cc1 ; c1ccc(-c2ccc(N(c3cc4c(oc5ccccc54)c(-c4ccccc4)c3-c3ccccc3)c3cc4ccccc4c4ccccc34)cc2)cc1]

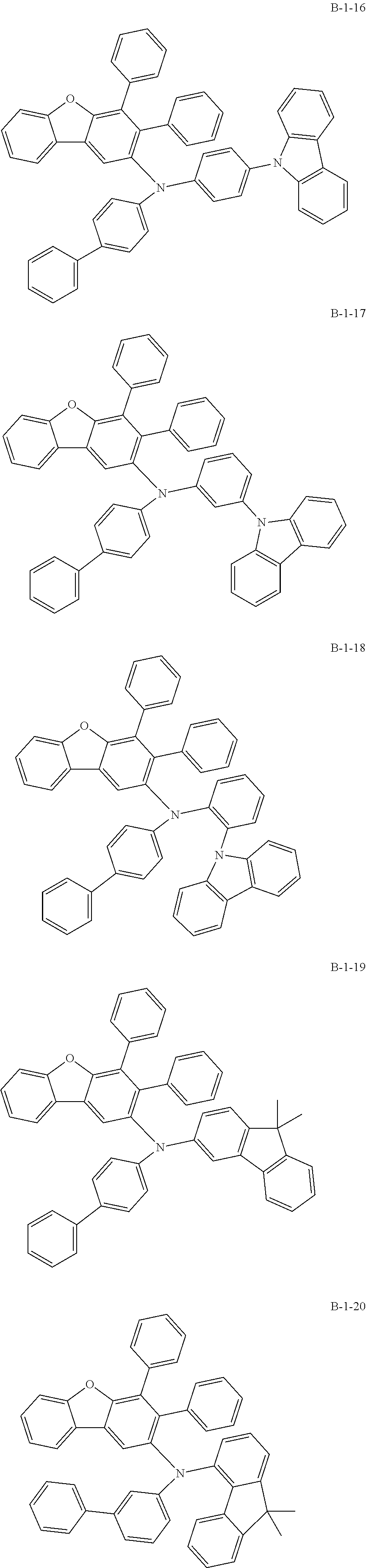 [SMILES: c1ccc(-c2ccc(N(c3ccc(-n4c5ccccc5c5ccccc54)cc3)c3cc4c(oc5ccccc54)c(-c4ccccc4)c3-c3ccccc3)cc2)cc1 ; c1ccc(-c2ccc(N(c3cccc(-n4c5ccccc5c5ccccc54)c3)c3cc4c(oc5ccccc54)c(-c4ccccc4)c3-c3ccccc3)cc2)cc1 ; c1ccc(-c2ccc(N(c3ccccc3-n3c4ccccc4c4ccccc43)c3cc4c(oc5ccccc54)c(-c4ccccc4)c3-c3ccccc3)cc2)cc1 ; CC1(C)c2ccccc2-c2cc(N(c3ccc(-c4ccccc4)cc3)c3cc4c(oc5ccccc54)c(-c4ccccc4)c3-c3ccccc3)ccc21 ; CC1(C)c2ccccc2-c2c(N(c3cccc(-c4ccccc4)c3)c3cc4c(oc5ccccc54)c(-c4ccccc4)c3-c3ccccc3)cccc21]

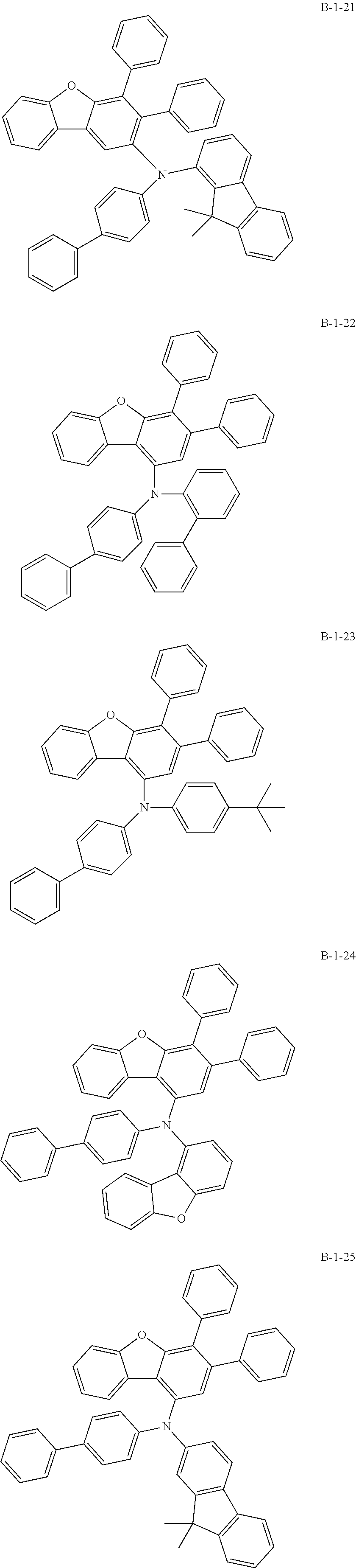 [SMILES: CC1(C)c2ccccc2-c2cccc(N(c3ccc(-c4ccccc4)cc3)c3cc4c(oc5ccccc54)c(-c4ccccc4)c3-c3ccccc3)c21 ; c1ccc(-c2ccc(N(c3ccccc3-c3ccccc3)c3cc(-c4ccccc4)c(-c4ccccc4)c4oc5ccccc5c34)cc2)cc1 ; CC(C)(C)c1ccc(N(c2ccc(-c3ccccc3)cc2)c2cc(-c3ccccc3)c(-c3ccccc3)c3oc4ccccc4c23)cc1 ; c1ccc(-c2ccc(N(c3cccc4oc5ccccc5c34)c3cc(-c4ccccc4)c(-c4ccccc4)c4oc5ccccc5c34)cc2)cc1 ; CC1(C)c2ccccc2-c2ccc(N(c3ccc(-c4ccccc4)cc3)c3cc(-c4ccccc4)c(-c4ccccc4)c4oc5ccccc5c34)cc21]

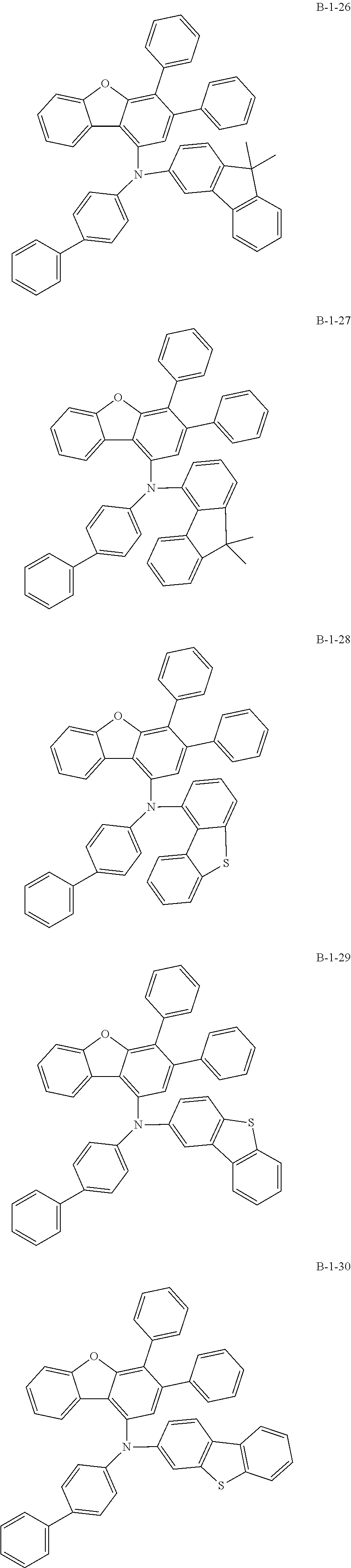 [SMILES: CC1(C)c2ccccc2-c2cc(N(c3ccc(-c4ccccc4)cc3)c3cc(-c4ccccc4)c(-c4ccccc4)c4oc5ccccc5c34)ccc21 ; CC1(C)c2ccccc2-c2c(N(c3ccc(-c4ccccc4)cc3)c3cc(-c4ccccc4)c(-c4ccccc4)c4oc5ccccc5c34)cccc21 ; c1ccc(-c2ccc(N(c3cccc4sc5ccccc5c34)c3cc(-c4ccccc4)c(-c4ccccc4)c4oc5ccccc5c34)cc2)cc1 ; c1ccc(-c2ccc(N(c3ccc4sc5ccccc5c4c3)c3cc(-c4ccccc4)c(-c4ccccc4)c4oc5ccccc5c34)cc2)cc1 ; c1ccc(-c2ccc(N(c3ccc4c(c3)sc3ccccc34)c3cc(-c4ccccc4)c(-c4ccccc4)c4oc5ccccc5c34)cc2)cc1]

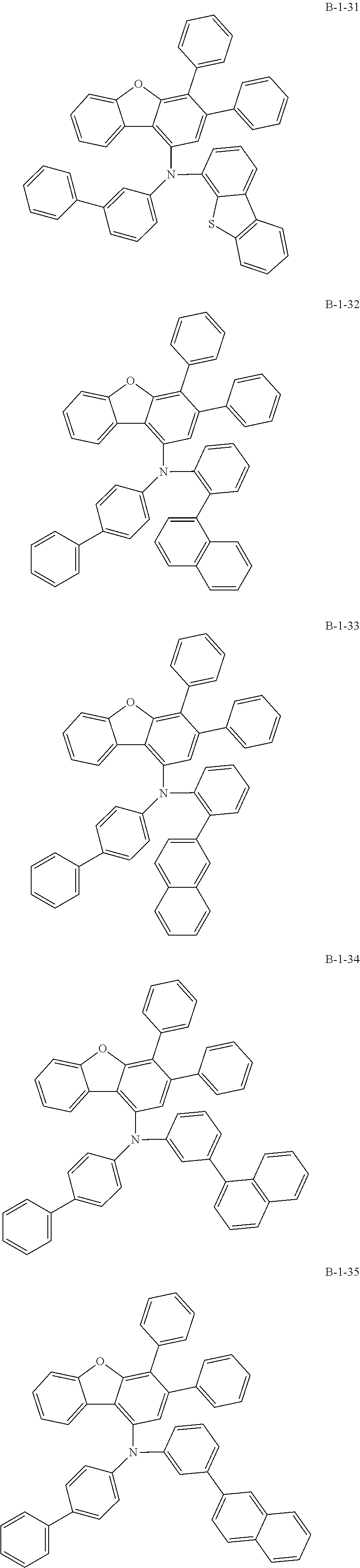 [SMILES: c1ccc(-c2cccc(N(c3cccc4c3sc3ccccc34)c3cc(-c4ccccc4)c(-c4ccccc4)c4oc5ccccc5c34)c2)cc1 ; c1ccc(-c2ccc(N(c3ccccc3-c3cccc4ccccc34)c3cc(-c4ccccc4)c(-c4ccccc4)c4oc5ccccc5c34)cc2)cc1 ; c1ccc(-c2ccc(N(c3ccccc3-c3ccc4ccccc4c3)c3cc(-c4ccccc4)c(-c4ccccc4)c4oc5ccccc5c34)cc2)cc1 ; c1ccc(-c2ccc(N(c3cccc(-c4cccc5ccccc45)c3)c3cc(-c4ccccc4)c(-c4ccccc4)c4oc5ccccc5c34)cc2)cc1 ; c1ccc(-c2ccc(N(c3cccc(-c4ccc5ccccc5c4)c3)c3cc(-c4ccccc4)c(-c4ccccc4)c4oc5ccccc5c34)cc2)cc1]

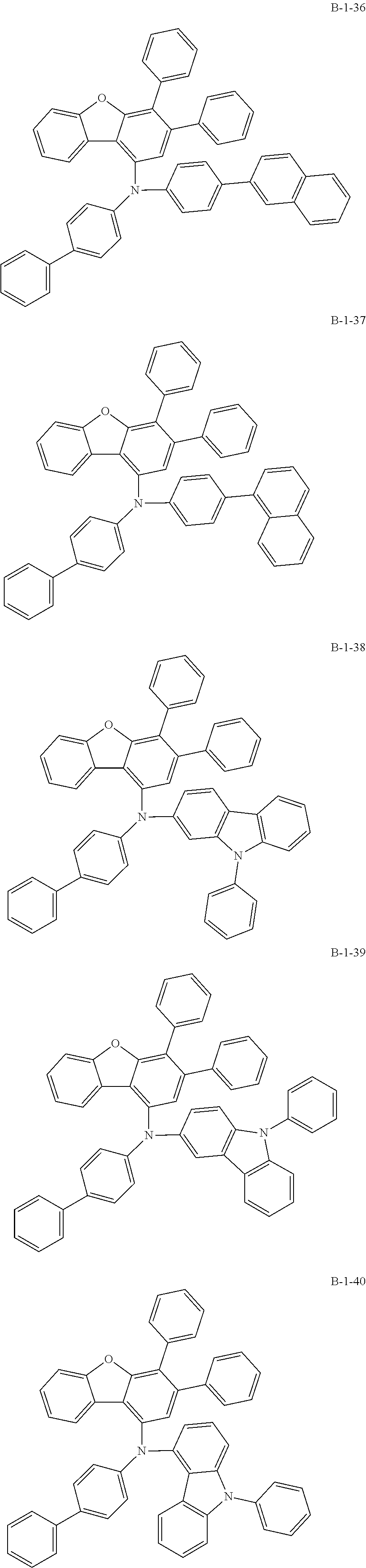 [SMILES: c1ccc(-c2ccc(N(c3ccc(-c4ccc5ccccc5c4)cc3)c3cc(-c4ccccc4)c(-c4ccccc4)c4oc5ccccc5c34)cc2)cc1 ; c1ccc(-c2ccc(N(c3ccc(-c4cccc5ccccc45)cc3)c3cc(-c4ccccc4)c(-c4ccccc4)c4oc5ccccc5c34)cc2)cc1 ; c1ccc(-c2ccc(N(c3ccc4c5ccccc5n(-c5ccccc5)c4c3)c3cc(-c4ccccc4)c(-c4ccccc4)c4oc5ccccc5c34)cc2)cc1 ; c1ccc(-c2ccc(N(c3ccc4c(c3)c3ccccc3n4-c3ccccc3)c3cc(-c4ccccc4)c(-c4ccccc4)c4oc5ccccc5c34)cc2)cc1 ; c1ccc(-c2ccc(N(c3cc(-c4ccccc4)c(-c4ccccc4)c4oc5ccccc5c34)c3cccc4c3c3ccccc3n4-c3ccccc3)cc2)cc1]

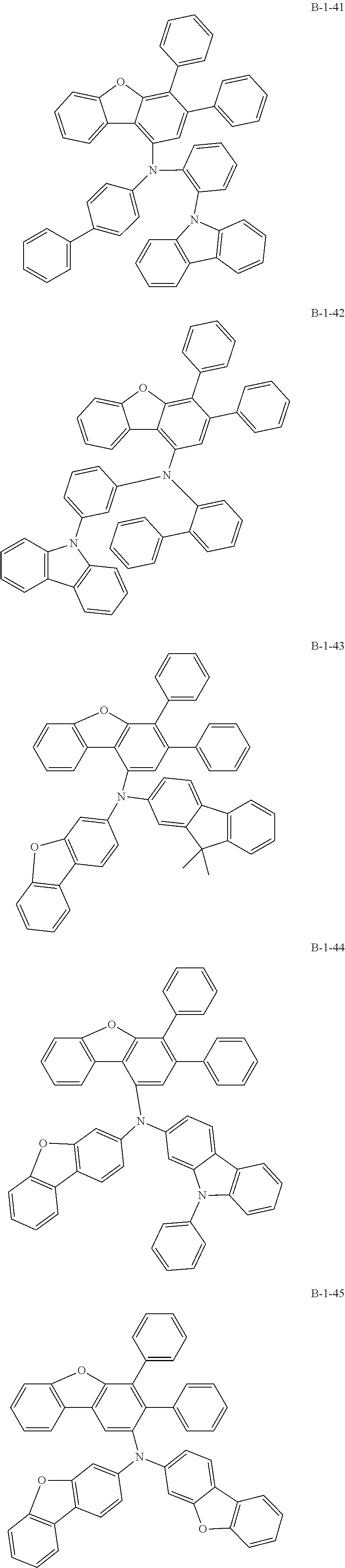 [SMILES: c1ccc(-c2ccc(N(c3ccccc3-n3c4ccccc4c4ccccc43)c3cc(-c4ccccc4)c(-c4ccccc4)c4oc5ccccc5c34)cc2)cc1 ; c1ccc(-c2ccccc2N(c2cccc(-n3c4ccccc4c4ccccc43)c2)c2cc(-c3ccccc3)c(-c3ccccc3)c3oc4ccccc4c23)cc1 ; CC1(C)c2ccccc2-c2ccc(N(c3ccc4c(c3)oc3ccccc34)c3cc(-c4ccccc4)c(-c4ccccc4)c4oc5ccccc5c34)cc21 ; c1ccc(-c2cc(N(c3ccc4c(c3)oc3ccccc34)c3ccc4c5ccccc5n(-c5ccccc5)c4c3)c3c(oc4ccccc43)c2-c2ccccc2)cc1 ; c1ccc(-c2c(N(c3ccc4c(c3)oc3ccccc34)c3ccc4c(c3)oc3ccccc34)cc3c(oc4ccccc43)c2-c2ccccc2)cc1]

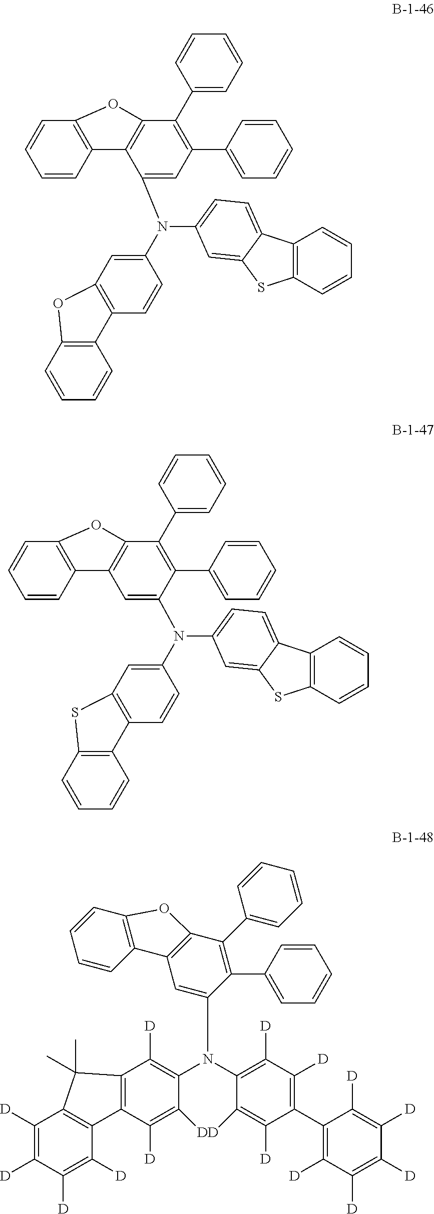 [SMILES: c1ccc(-c2cc(N(c3ccc4c(c3)oc3ccccc34)c3ccc4c(c3)sc3ccccc34)c3c(oc4ccccc43)c2-c2ccccc2)cc1 ; c1ccc(-c2c(N(c3ccc4c(c3)sc3ccccc34)c3ccc4c(c3)sc3ccccc34)cc3c(oc4ccccc43)c2-c2ccccc2)cc1 ; [2H]c1c([2H])c([2H])c(-c2c([2H])c([2H])c(N(c3cc4c(oc5ccccc54)c(-c4ccccc4)c3-c3ccccc3)c3c([2H])c([2H])c4c(c3[2H])C(C)(C)c3c([2H])c([2H])c([2H])c([2H])c3-4)c([2H])c2[2H])c([2H])c1[2H]]

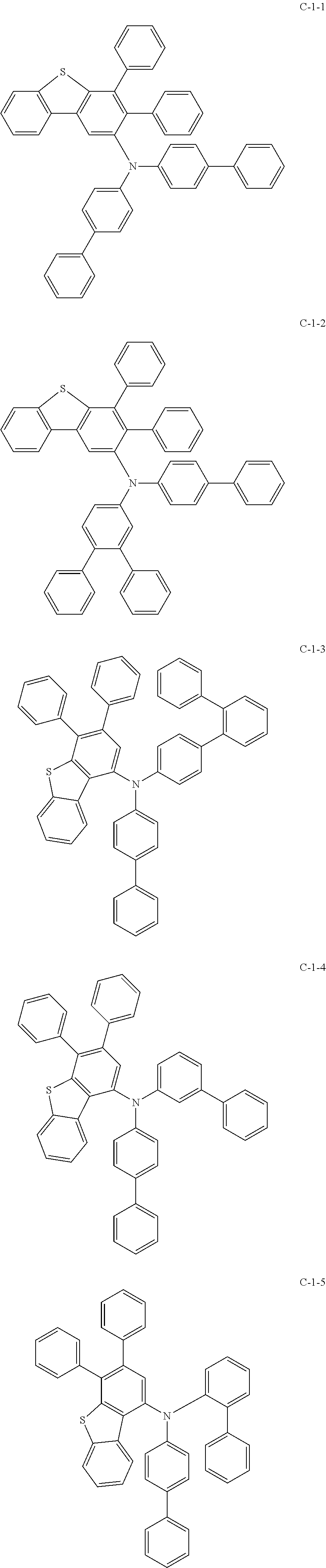 [SMILES: c1ccc(-c2ccc(N(c3ccc(-c4ccccc4)cc3)c3cc4c(sc5ccccc54)c(-c4ccccc4)c3-c3ccccc3)cc2)cc1 ; c1ccc(-c2ccc(N(c3ccc(-c4ccccc4)c(-c4ccccc4)c3)c3cc4c(sc5ccccc54)c(-c4ccccc4)c3-c3ccccc3)cc2)cc1 ; c1ccc(-c2ccc(N(c3ccc(-c4ccccc4-c4ccccc4)cc3)c3cc(-c4ccccc4)c(-c4ccccc4)c4sc5ccccc5c34)cc2)cc1 ; c1ccc(-c2ccc(N(c3cccc(-c4ccccc4)c3)c3cc(-c4ccccc4)c(-c4ccccc4)c4sc5ccccc5c34)cc2)cc1 ; c1ccc(-c2ccc(N(c3ccccc3-c3ccccc3)c3cc(-c4ccccc4)c(-c4ccccc4)c4sc5ccccc5c34)cc2)cc1]

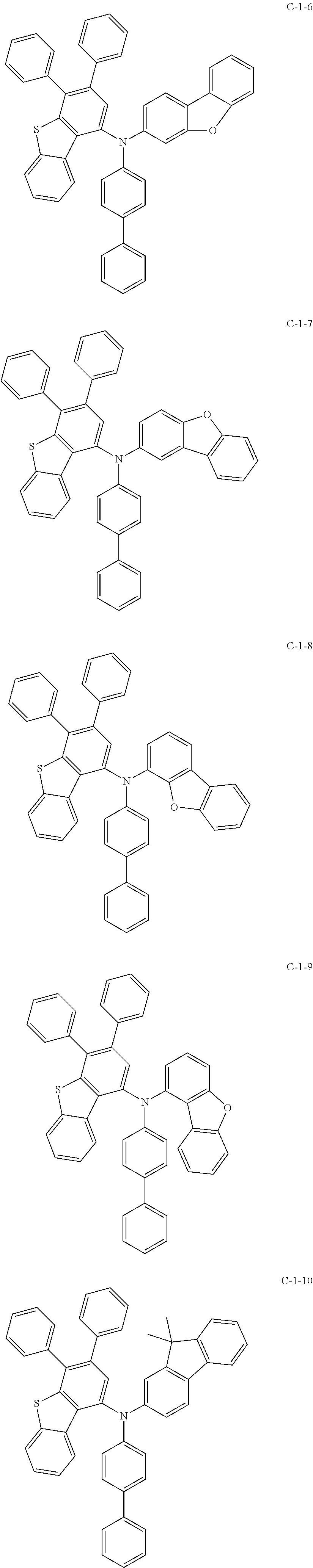 [SMILES: c1ccc(-c2ccc(N(c3ccc4c(c3)oc3ccccc34)c3cc(-c4ccccc4)c(-c4ccccc4)c4sc5ccccc5c34)cc2)cc1 ; c1ccc(-c2ccc(N(c3ccc4oc5ccccc5c4c3)c3cc(-c4ccccc4)c(-c4ccccc4)c4sc5ccccc5c34)cc2)cc1 ; c1ccc(-c2ccc(N(c3cccc4c3oc3ccccc34)c3cc(-c4ccccc4)c(-c4ccccc4)c4sc5ccccc5c34)cc2)cc1 ; c1ccc(-c2ccc(N(c3cccc4oc5ccccc5c34)c3cc(-c4ccccc4)c(-c4ccccc4)c4sc5ccccc5c34)cc2)cc1 ; CC1(C)c2ccccc2-c2ccc(N(c3ccc(-c4ccccc4)cc3)c3cc(-c4ccccc4)c(-c4ccccc4)c4sc5ccccc5c34)cc21]

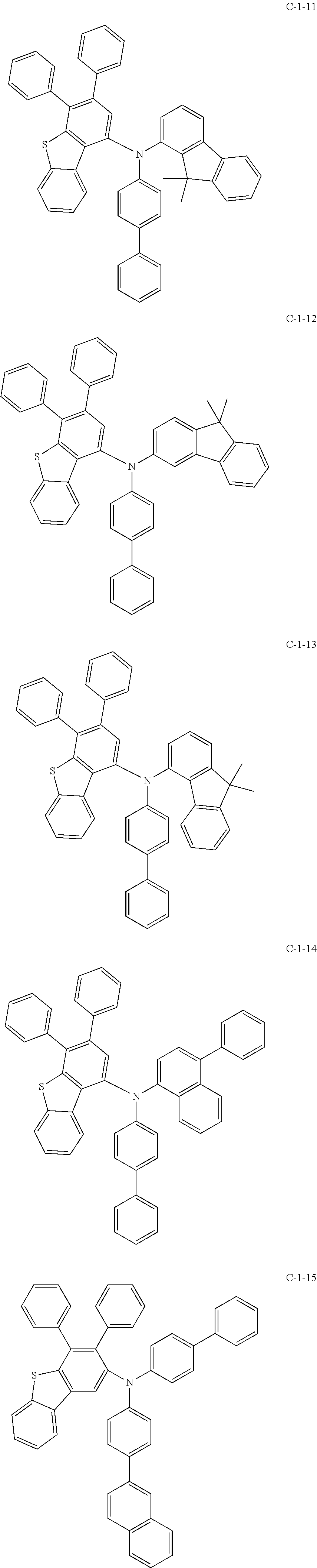 [SMILES: CC1(C)c2ccccc2-c2cccc(N(c3ccc(-c4ccccc4)cc3)c3cc(-c4ccccc4)c(-c4ccccc4)c4sc5ccccc5c34)c21 ; CC1(C)c2ccccc2-c2cc(N(c3ccc(-c4ccccc4)cc3)c3cc(-c4ccccc4)c(-c4ccccc4)c4sc5ccccc5c34)ccc21 ; CC1(C)c2ccccc2-c2c(N(c3ccc(-c4ccccc4)cc3)c3cc(-c4ccccc4)c(-c4ccccc4)c4sc5ccccc5c34)cccc21 ; c1ccc(-c2ccc(N(c3ccc(-c4ccccc4)c4ccccc34)c3cc(-c4ccccc4)c(-c4ccccc4)c4sc5ccccc5c34)cc2)cc1 ; c1ccc(-c2ccc(N(c3ccc(-c4ccc5ccccc5c4)cc3)c3cc4c(sc5ccccc54)c(-c4ccccc4)c3-c3ccccc3)cc2)cc1]

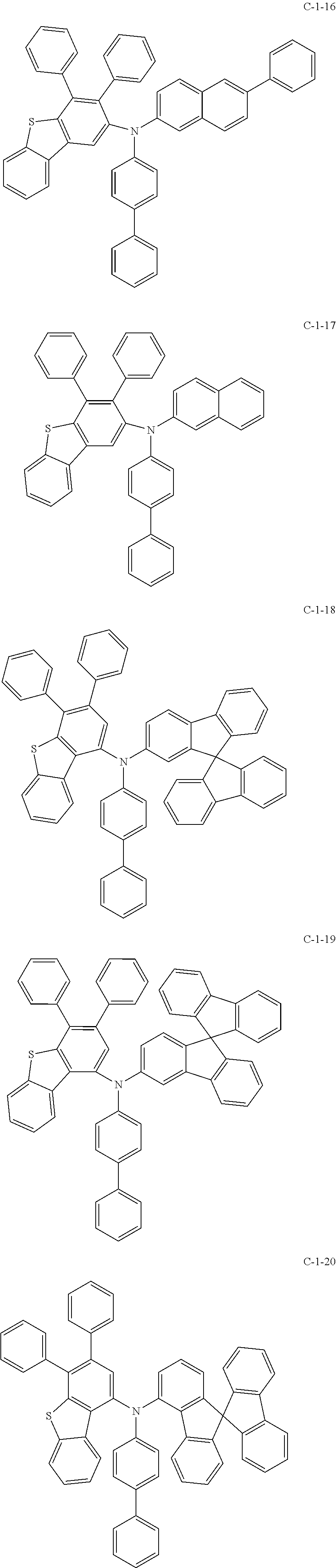 [SMILES: c1ccc(-c2ccc(N(c3ccc4cc(-c5ccccc5)ccc4c3)c3cc4c(sc5ccccc54)c(-c4ccccc4)c3-c3ccccc3)cc2)cc1 ; c1ccc(-c2ccc(N(c3ccc4ccccc4c3)c3cc4c(sc5ccccc54)c(-c4ccccc4)c3-c3ccccc3)cc2)cc1 ; c1ccc(-c2ccc(N(c3ccc4c(c3)C3(c5ccccc5-c5ccccc53)c3ccccc3-4)c3cc(-c4ccccc4)c(-c4ccccc4)c4sc5ccccc5c34)cc2)cc1 ; c1ccc(-c2ccc(N(c3ccc4c(c3)-c3ccccc3C43c4ccccc4-c4ccccc43)c3cc(-c4ccccc4)c(-c4ccccc4)c4sc5ccccc5c34)cc2)cc1 ; c1ccc(-c2ccc(N(c3cccc4c3-c3ccccc3C43c4ccccc4-c4ccccc43)c3cc(-c4ccccc4)c(-c4ccccc4)c4sc5ccccc5c34)cc2)cc1]

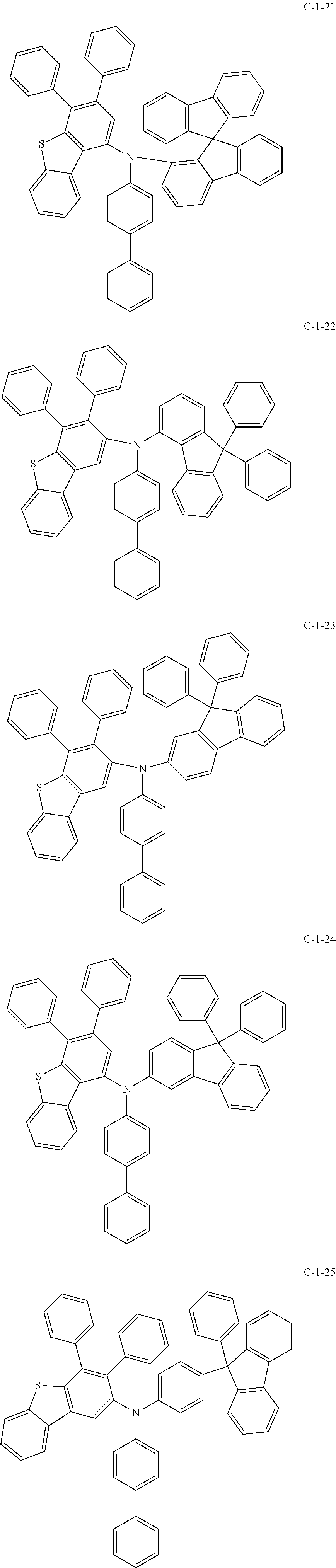

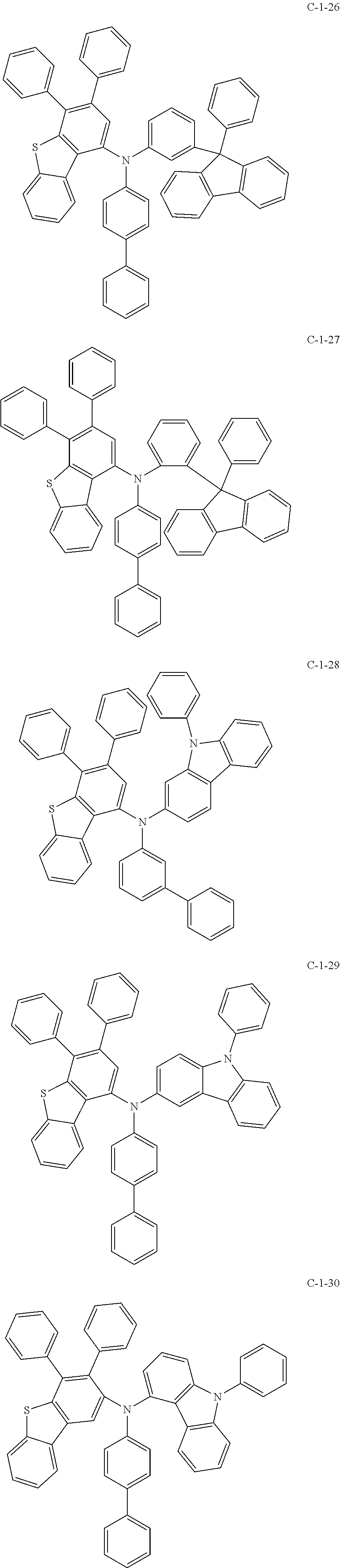

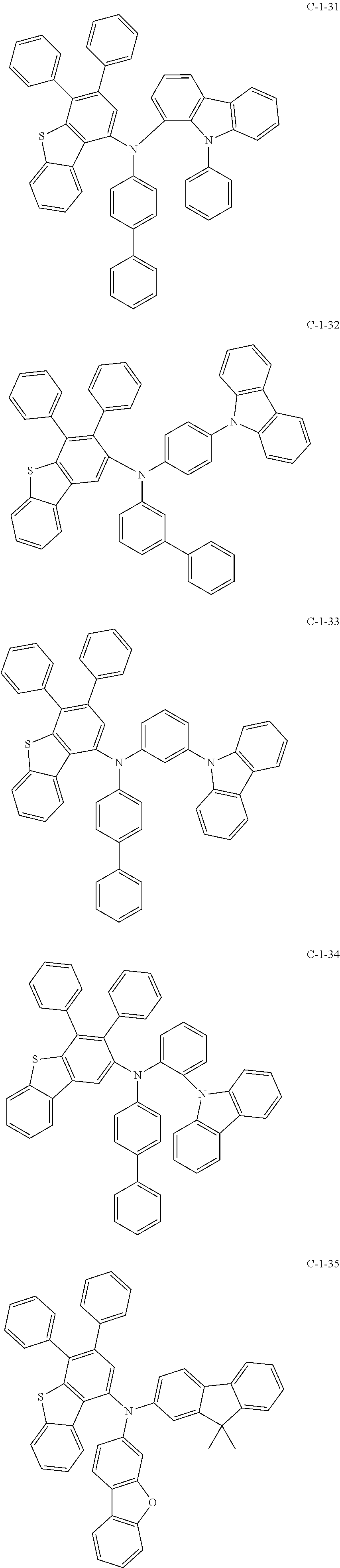 [SMILES: c1ccc(-c2ccc(N(c3cc(-c4ccccc4)c(-c4ccccc4)c4sc5ccccc5c34)c3cccc4c5ccccc5n(-c5ccccc5)c34)cc2)cc1 ; c1ccc(-c2cccc(N(c3ccc(-n4c5ccccc5c5ccccc54)cc3)c3cc4c(sc5ccccc54)c(-c4ccccc4)c3-c3ccccc3)c2)cc1 ; c1ccc(-c2ccc(N(c3cccc(-n4c5ccccc5c5ccccc54)c3)c3cc(-c4ccccc4)c(-c4ccccc4)c4sc5ccccc5c34)cc2)cc1 ; c1ccc(-c2ccc(N(c3ccccc3-n3c4ccccc4c4ccccc43)c3cc4c(sc5ccccc54)c(-c4ccccc4)c3-c3ccccc3)cc2)cc1 ; CC1(C)c2ccccc2-c2ccc(N(c3ccc4c(c3)oc3ccccc34)c3cc(-c4ccccc4)c(-c4ccccc4)c4sc5ccccc5c34)cc21]

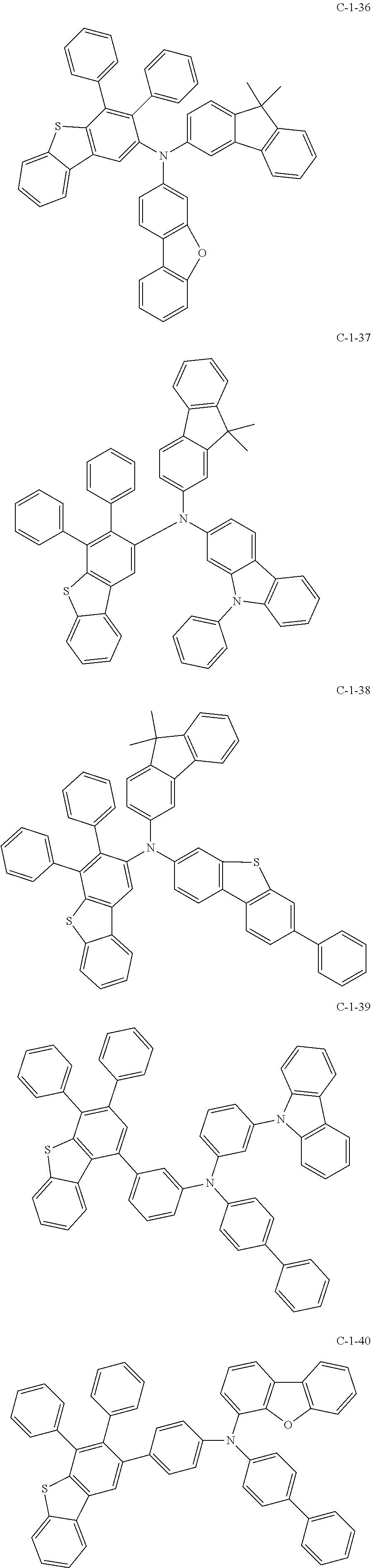

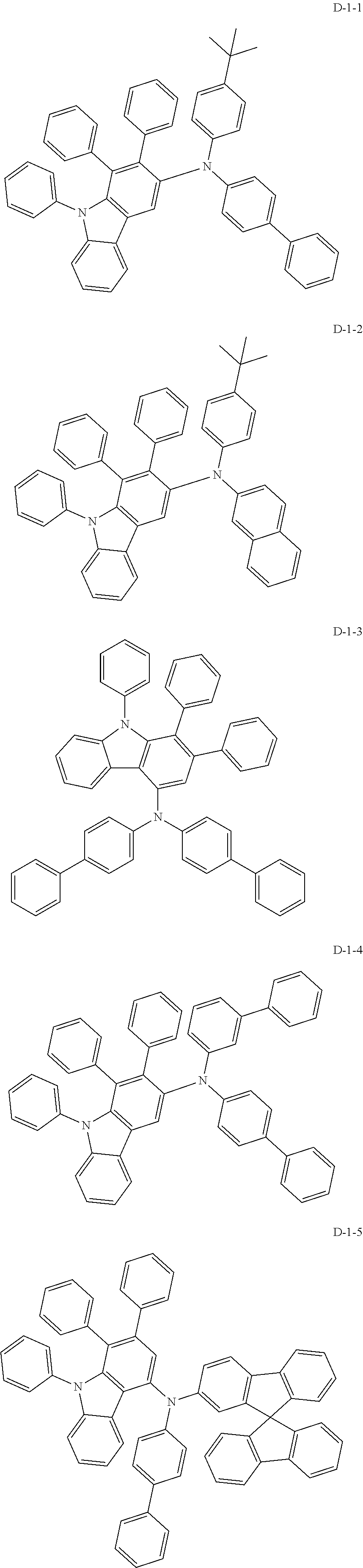

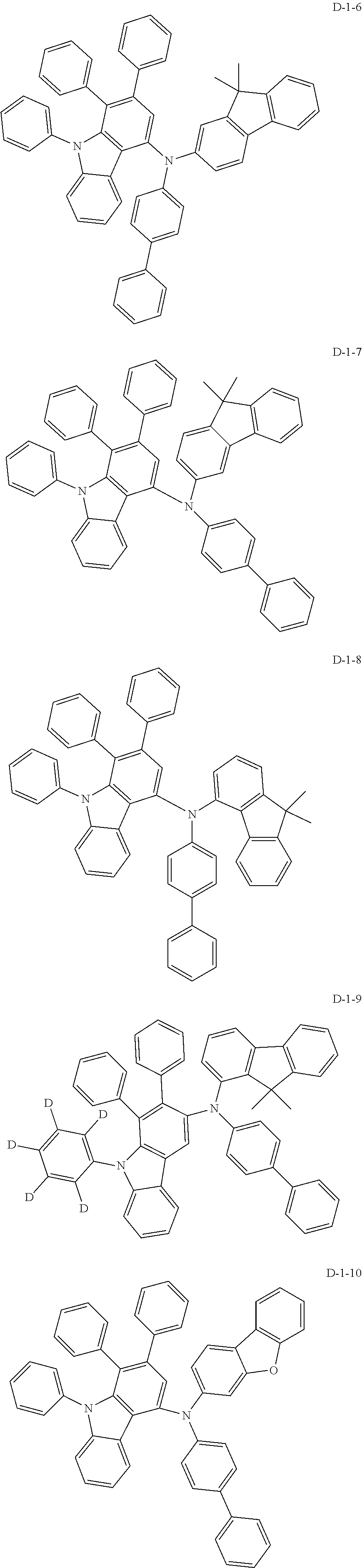

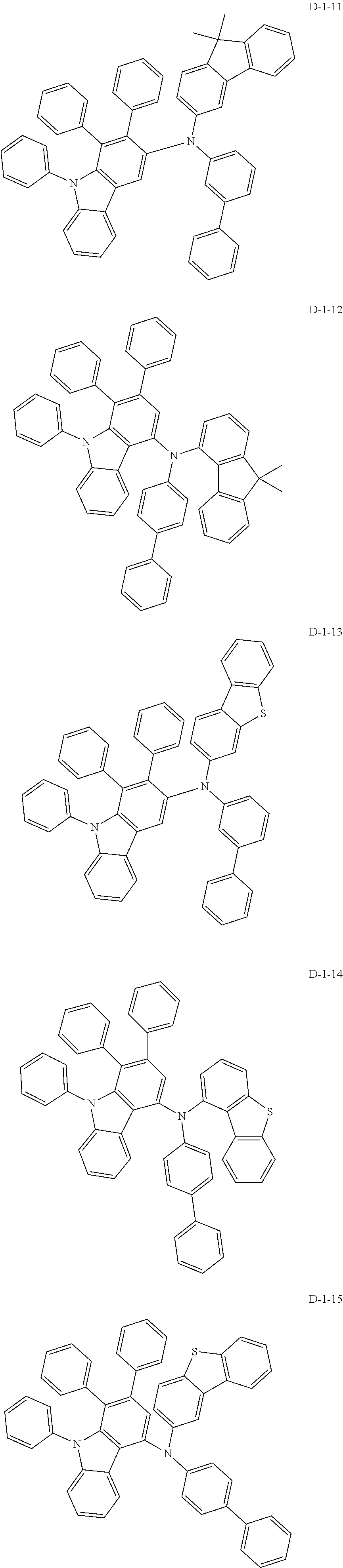 [SMILES: CC1(C)c2ccccc2-c2cc(N(c3cccc(-c4ccccc4)c3)c3cc4c5ccccc5n(-c5ccccc5)c4c(-c4ccccc4)c3-c3ccccc3)ccc21 ; CC1(C)c2ccccc2-c2c(N(c3ccc(-c4ccccc4)cc3)c3cc(-c4ccccc4)c(-c4ccccc4)c4c3c3ccccc3n4-c3ccccc3)cccc21 ; c1ccc(-c2cccc(N(c3ccc4c(c3)sc3ccccc34)c3cc4c5ccccc5n(-c5ccccc5)c4c(-c4ccccc4)c3-c3ccccc3)c2)cc1 ; c1ccc(-c2ccc(N(c3cccc4sc5ccccc5c34)c3cc(-c4ccccc4)c(-c4ccccc4)c4c3c3ccccc3n4-c3ccccc3)cc2)cc1 ; c1ccc(-c2ccc(N(c3ccc4sc5ccccc5c4c3)c3cc(-c4ccccc4)c(-c4ccccc4)c4c3c3ccccc3n4-c3ccccc3)cc2)cc1]

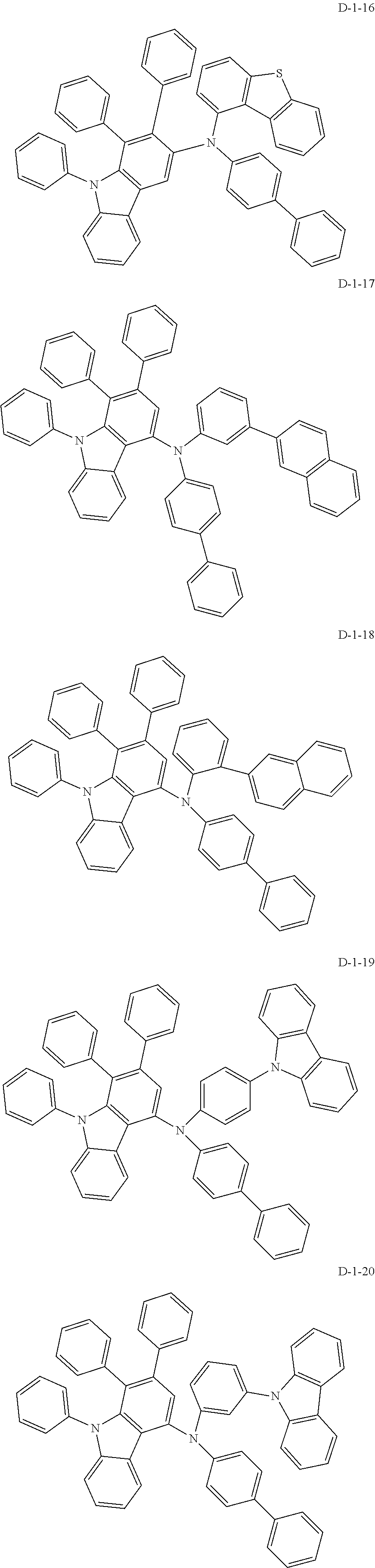

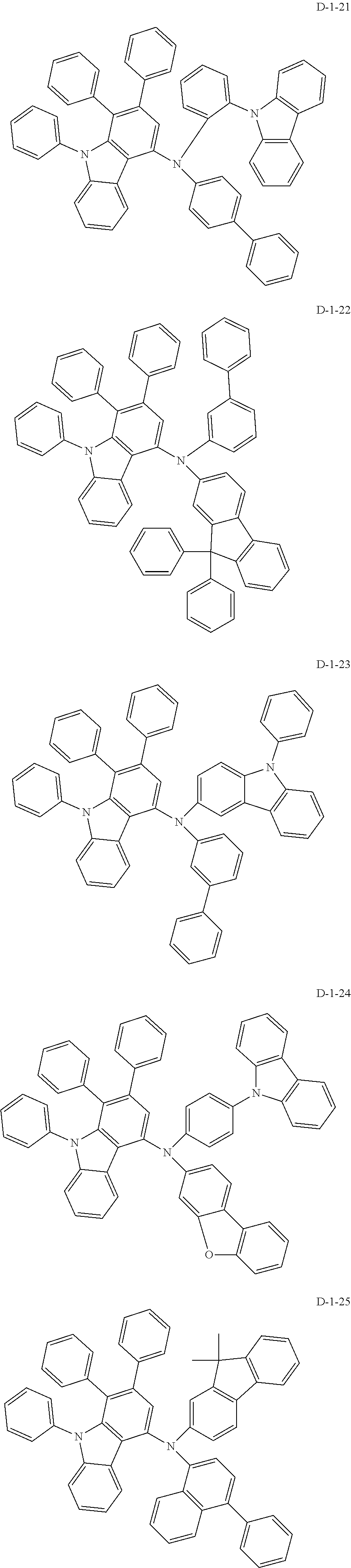

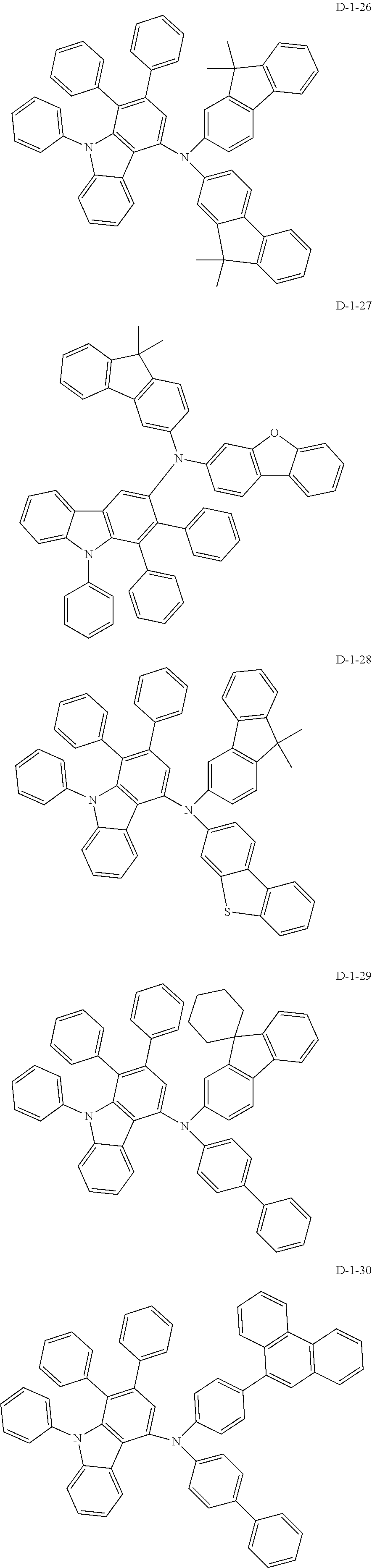

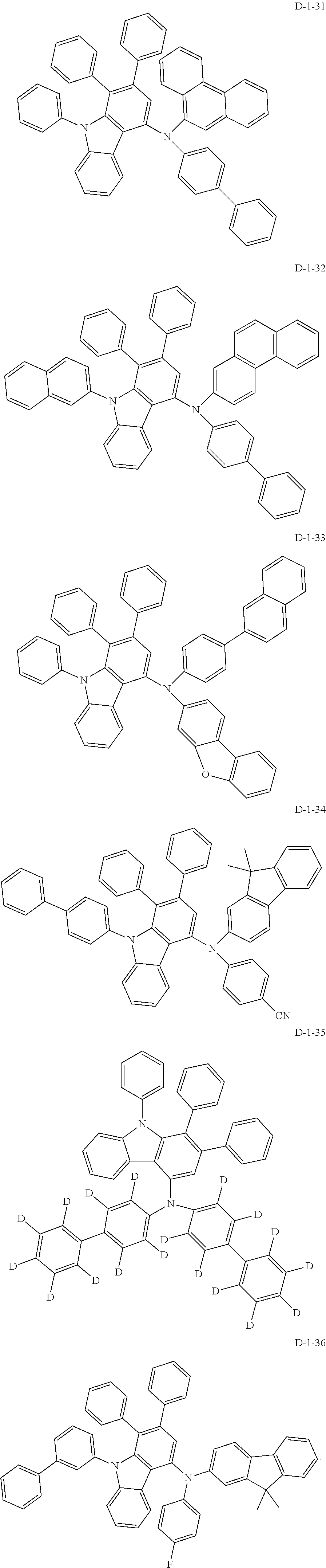

In a second aspect, the present application provides an electronic element, comprising an anode and a cathode disposed opposite each other, and a functional layer disposed between the anode and the cathode; wherein the functional layer comprises the organic compounds of the present application.

Alternatively, the functional layer comprises a hole transport layer, the hole transport layer comprising an organic compound of the present application.

Alternatively, the electronic element is an organic electroluminescent device or a photoelectric conversion device. Further alternatively, the organic electroluminescent device is a red-light organic electroluminescent device.

Further alternatively, the hole transport layer comprises a first hole transport layer and a second hole transport layer, the first hole transport layer being closer to the anode with respect to the second hole transport layer, wherein the second hole transport layer comprises an organic compound of the present application.

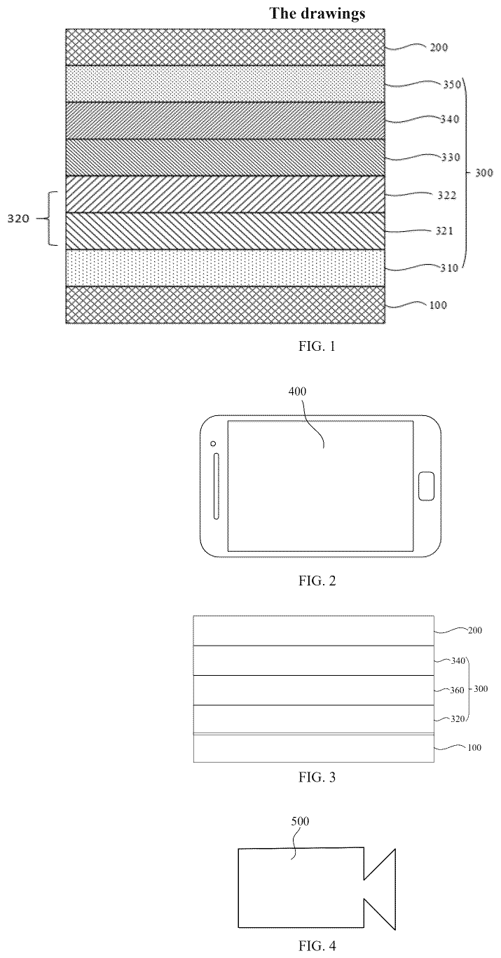

In one embodiment, the electronic element is an organic electroluminescent device. As shown in FIG. 1, the organic electroluminescent device may include an anode 100, a first hole transport layer 321, a second bole transport layer 322, an organic light-emitting layer 330, an electron transport layer 340 and a cathode 200 that are stacked. Among them, the first hole transport layer 321 and the second hole transport layer 322 constitute the hole transport layer 320.

Alternatively, the anode 100 comprises an anode material below, which is preferably a high work function material contributing to injection of holes into the functional layer. The specific examples of the anode material include: metals such as nickel, platinum, vanadium, chromium, copper, zinc, and gold, or alloys thereof; metal oxides such as zinc oxide, indium oxide, indium tin oxide (ITO), and indium zinc oxide (IZO); combinations of metals and oxides, such as ZnO:Al or SnO₂:Sb; or conductive polymers such as poly(3-methylthiophene), poly[3,4-(ethylene-1,2-dioxy)thiophene] (PEDT), polypyrrole, and polyaniline, but are not limited thereto. Preferably, a transparent electrode comprising indium tin oxide (TTO) as the anode is included.

Alternatively, the hole transport layer comprises one or more hole transport materials, and the hole transport materials may be selected from carbazole multimers, carbazole-linked triarylamine based compounds, or other types of compounds, which are not specifically limited in the present application. For example, the material of the first hole transport layer is selected from the group consisting of the following compounds:

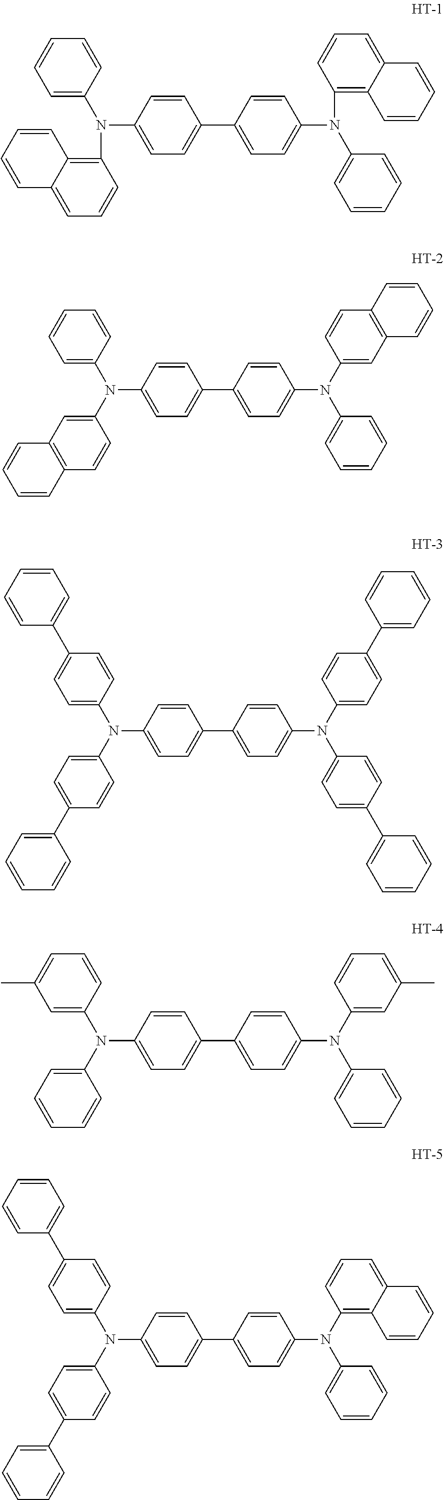

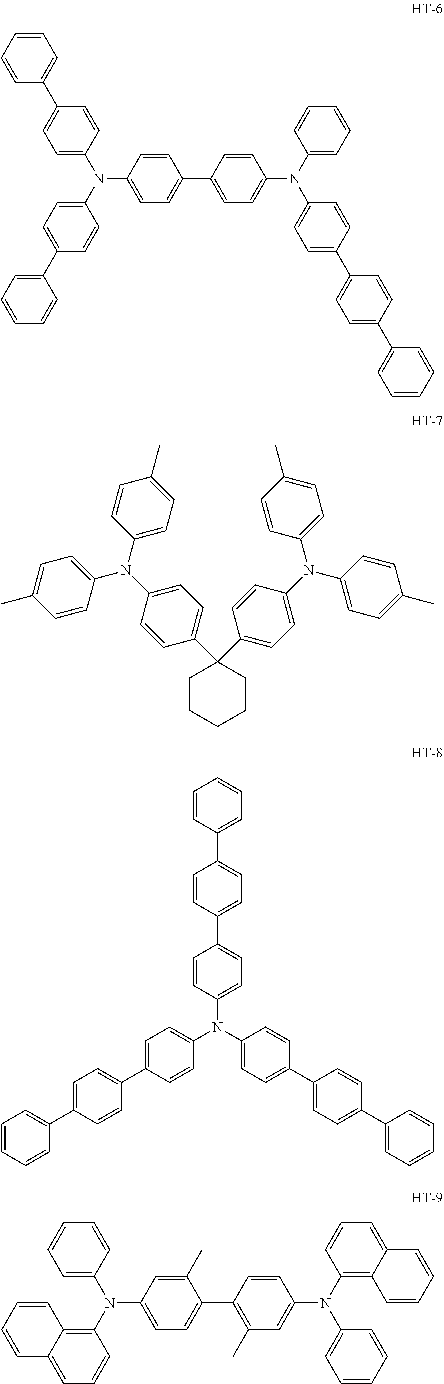

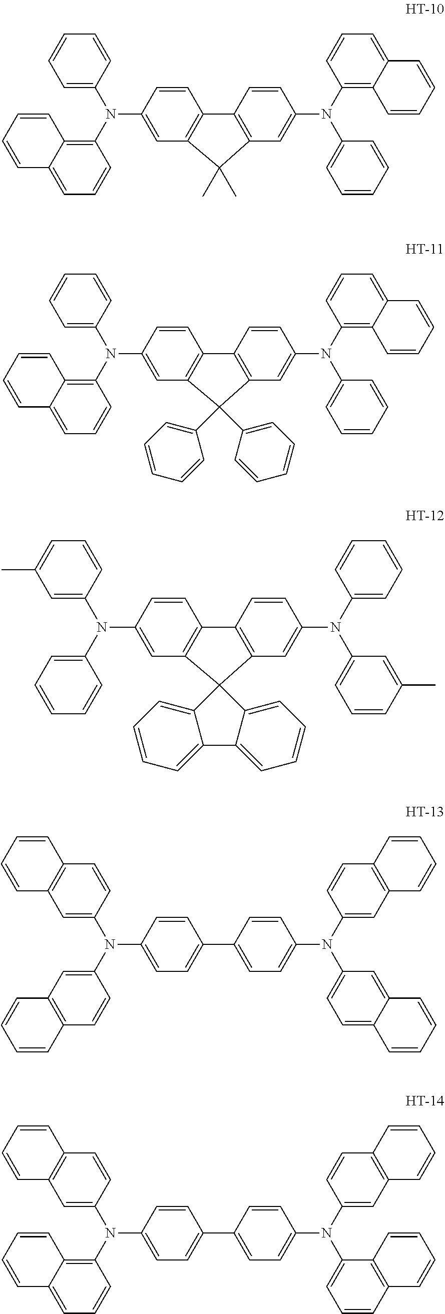

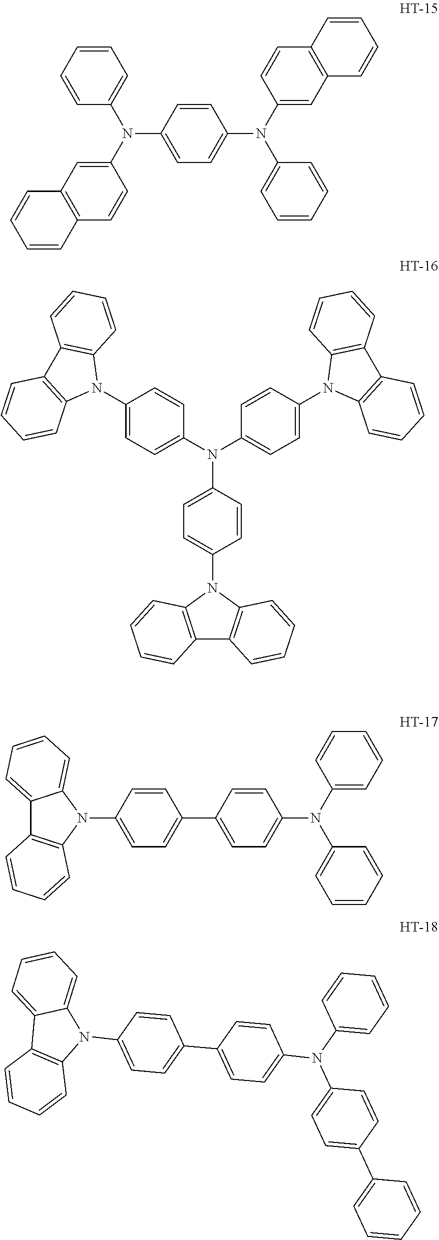

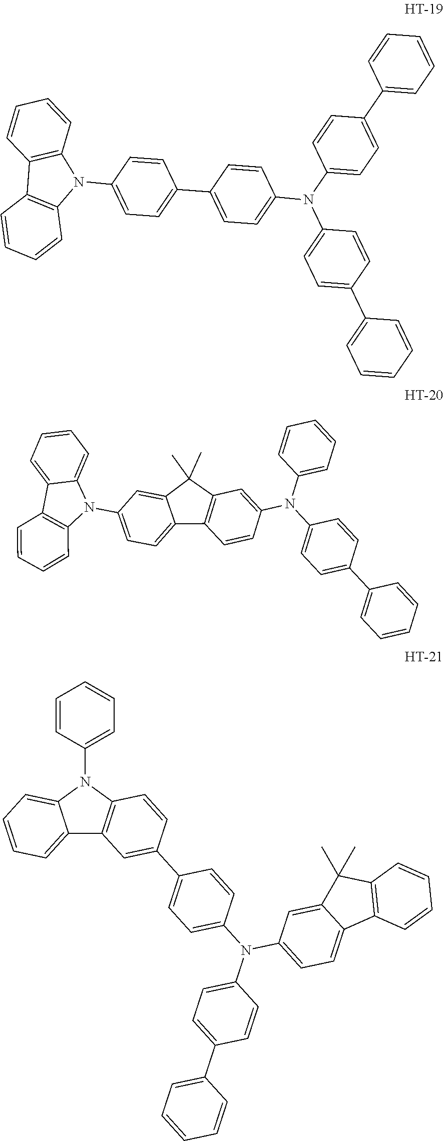 [SMILES: c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2cc(N(c3ccccc3)c3ccc(-c4ccccc4)cc3)ccc2-c2ccc(-n3c4ccccc4c4ccccc43)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21]

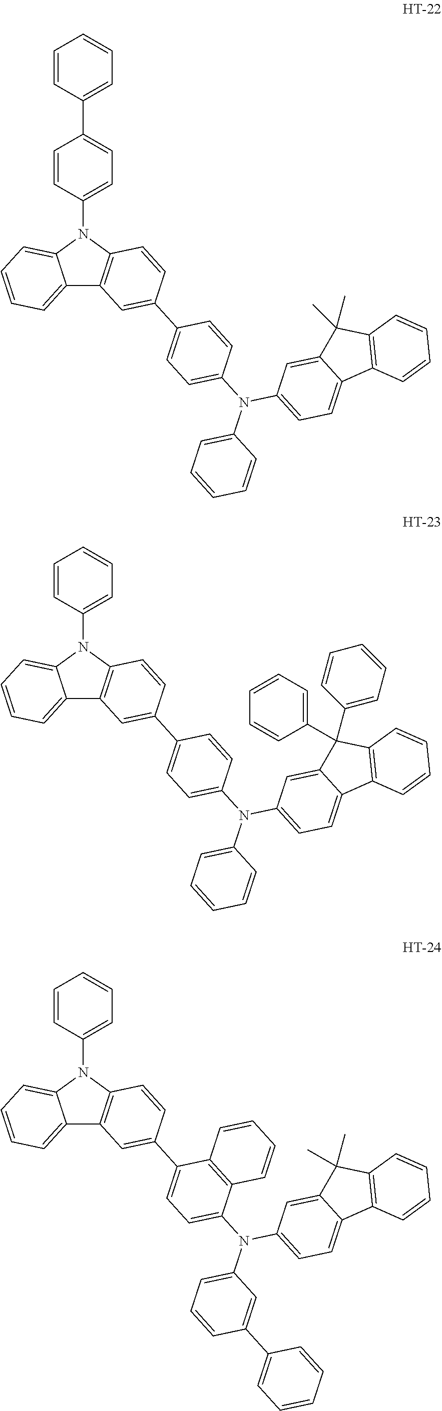 [SMILES: CC1(C)c2ccccc2-c2ccc(N(c3ccccc3)c3ccc(-c4ccc5c(c4)c4ccccc4n5-c4ccc(-c5ccccc5)cc4)cc3)cc21 ; c1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1 ; CC1(C)c2ccccc2-c2ccc(N(c3cccc(-c4ccccc4)c3)c3ccc(-c4ccc5c(c4)c4ccccc4n5-c4ccccc4)c4ccccc34)cc21]

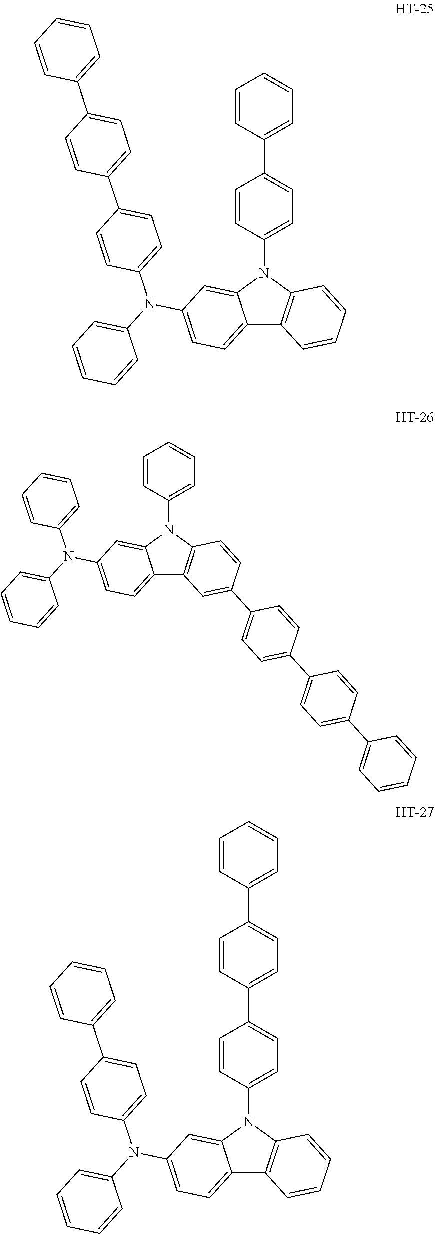 [SMILES: c1ccc(-c2ccc(-c3ccc(N(c4ccccc4)c4ccc5c6ccccc6n(-c6ccc(-c7ccccc7)cc6)c5c4)cc3)cc2)cc1 ; c1ccc(-c2ccc(-c3ccc(-c4ccc5c(c4)c4ccc(N(c6ccccc6)c6ccccc6)cc4n5-c4ccccc4)cc3)cc2)cc1 ; c1ccc(-c2ccc(-c3ccc(-n4c5ccccc5c5ccc(N(c6ccccc6)c6ccc(-c7ccccc7)cc6)cc54)cc3)cc2)cc1]

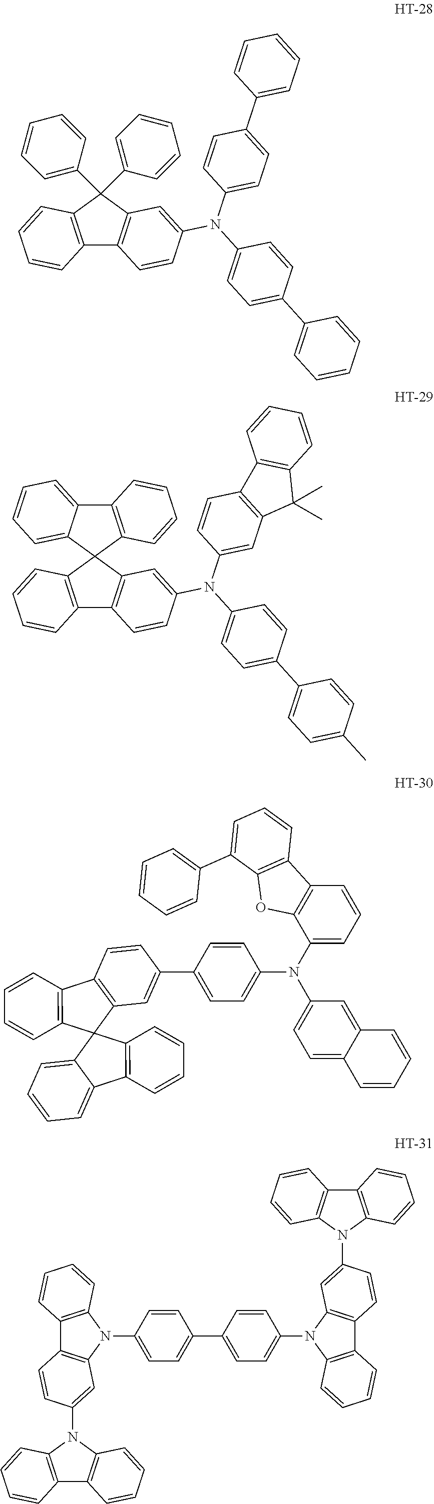

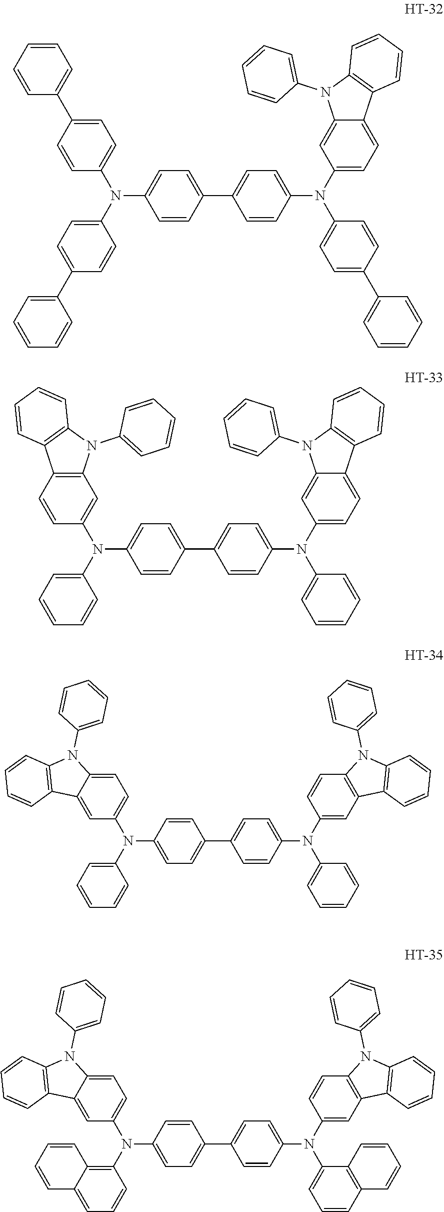 [SMILES: c1ccc(-c2ccc(N(c3ccc(-c4ccccc4)cc3)c3ccc(-c4ccc(N(c5ccc(-c6ccccc6)cc5)c5ccc6c7ccccc7n(-c7ccccc7)c6c5)cc4)cc3)cc2)cc1 ; c1ccc(N(c2ccc(-c3ccc(N(c4ccccc4)c4ccc5c6ccccc6n(-c6ccccc6)c5c4)cc3)cc2)c2ccc3c4ccccc4n(-c4ccccc4)c3c2)cc1 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; c1ccc(-n2c3ccccc3c3cc(N(c4ccc(-c5ccc(N(c6ccc7c(c6)c6ccccc6n7-c6ccccc6)c6cccc7ccccc67)cc5)cc4)c4cccc5ccccc45)ccc32)cc1]

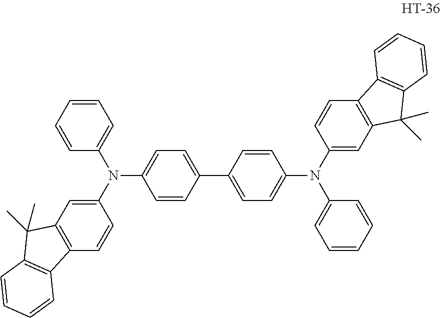 [SMILES: CC1(C)c2ccccc2-c2ccc(N(c3ccccc3)c3ccc(-c4ccc(N(c5ccccc5)c5ccc6c(c5)C(C)(C)c5ccccc5-6)cc4)cc3)cc21]

In one specific embodiment, the first hole transport layer 321 is compound HT-21.

Alternatively, the second hole transport layer 322 is an organic compound of the present application.

Alternatively, the organic light-emitting layer 330 may be composed of a single light-emitting material, or may comprise a host material and a guest material. Alternatively, the organic light-emitting layer 330 is composed of a host material and a guest material. The holes injected into the organic light-emitting layer 330 and the electrons injected into the organic light-emitting layer 330 can recombine in the organic light-emitting layer 330 to form excitons. The excitons transmit energy to the host material, and the host material transmits the energy to the guest material, thereby enabling the guest material to emit light.

The host material of the organic light-emitting layer 330 may be metal chelating compounds, stilbene-based derivatives, aromatic amine derivatives, dibenzofuran derivatives, or other types of materials, which are not specifically limited in the present application. The host material may be a single host material or a mixture of host materials. In one embodiment of the present application, the host material of the organic light-emitting layer 330 is CBP.

The guest material of the organic light-emitting layer 330 may be selected with reference to the prior art, and may be selected, for example, from an iridium (III) organometallic complex, a platinum (II) organometallic complex, a ruthenium (II) complex, and the like. Specific examples of the guest material include but are not limited to.

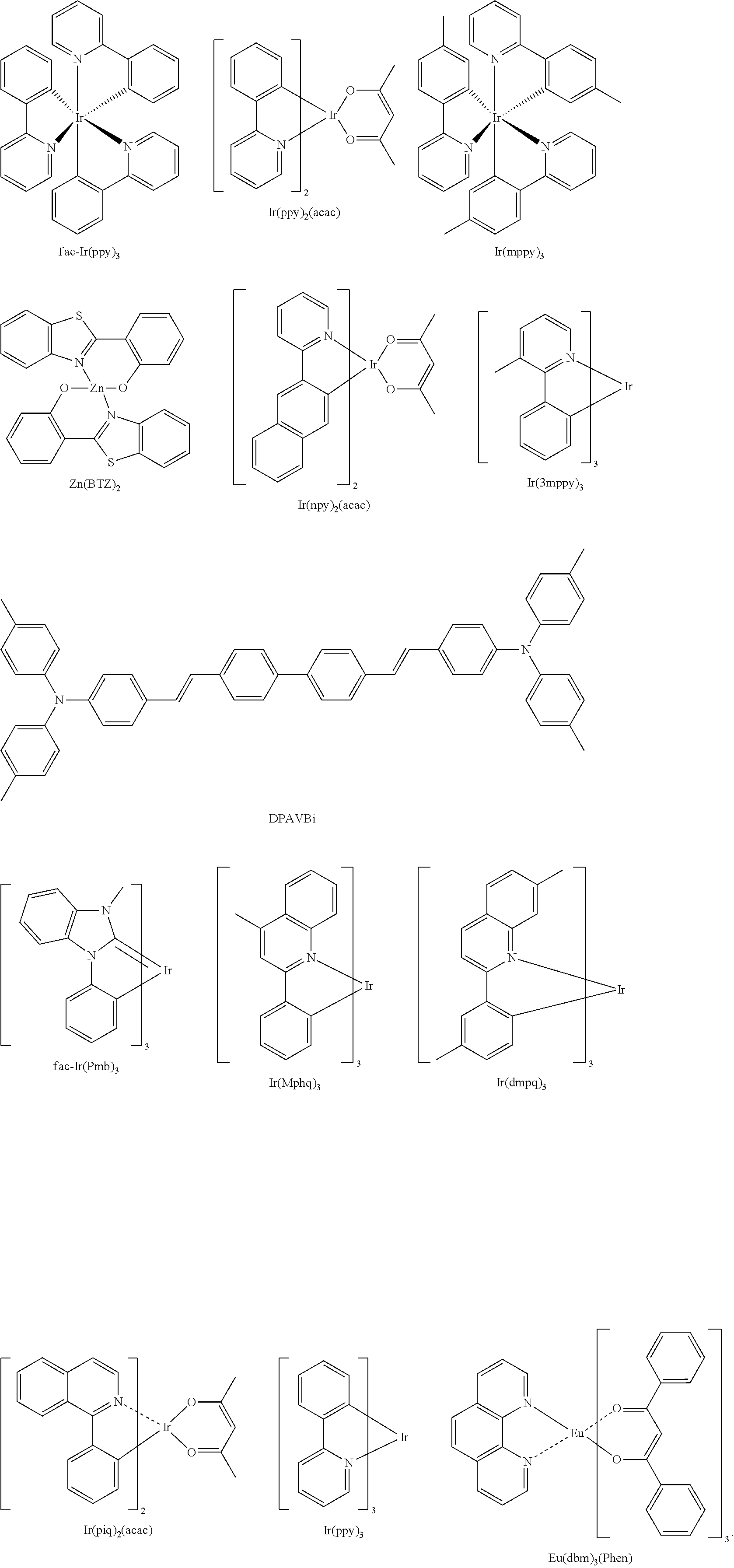

In one embodiment of the present application, the guest material of the organic light-emitting layer 330 is Ir(piq)₂(acac).

Alternatively, the electron transport layer 340 may be a single-layer structure or a multilayer structure, which may comprise one or more electron transport materials, and the electron transport material may typically comprise a metal complex and/or a nitrogen-containing heterocyclic derivative, wherein the metal complex material may be selected, for example, from LiQ, Alq₃ and the like; the nitrogen-containing heterocyclic derivative may be an aromatic ring having a nitrogen-containing six-membered ring or five-membered ring skeleton, a fused aromatic ring compound having a nitrogen-containing six-membered ring or five-membered ring skeleton, and the like; specific examples include, but are not limited to, 1,10-phenanthroline based compounds such as Bphen, NBphen, ET-21, BimiBphen; or anthracene based, triazine based or pyrimidine based compounds containing heteroaryl containing nitrogen having the structure shown below. In one embodiment of the present application, the electronic transport layer 340 is composed of ET-21 and LiQ.

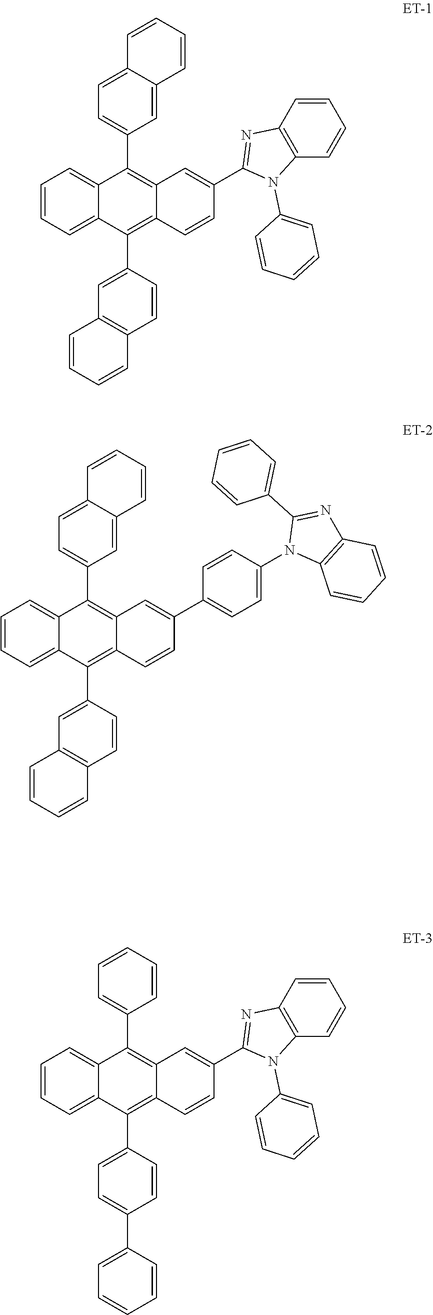 [SMILES: c1ccc(-n2c(-c3ccc4c(-c5ccc6ccccc6c5)c5ccccc5c(-c5ccc6ccccc6c5)c4c3)nc3ccccc32)cc1 ; c1ccc(-c2nc3ccccc3n2-c2ccc(-c3ccc4c(-c5ccc6ccccc6c5)c5ccccc5c(-c5ccc6ccccc6c5)c4c3)cc2)cc1 ; c1ccc(-c2ccc(-c3c4ccccc4c(-c4ccccc4)c4cc(-c5nc6ccccc6n5-c5ccccc5)ccc34)cc2)cc1]

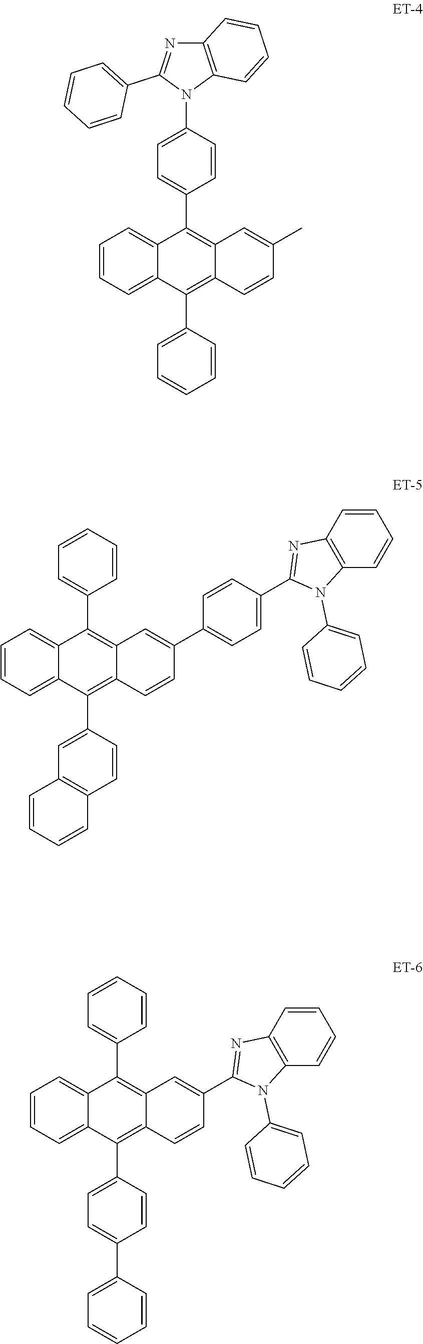 [SMILES: Cc1ccc2c(-c3ccccc3)c3ccccc3c(-c3ccc(-n4c(-c5ccccc5)nc5ccccc54)cc3)c2c1 ; c1ccc(-c2c3ccccc3c(-c3ccc4ccccc4c3)c3ccc(-c4ccc(-c5nc6ccccc6n5-c5ccccc5)cc4)cc23)cc1 ; c1ccc(-c2ccc(-c3c4ccccc4c(-c4ccccc4)c4cc(-c5nc6ccccc6n5-c5ccccc5)ccc34)cc2)cc1]

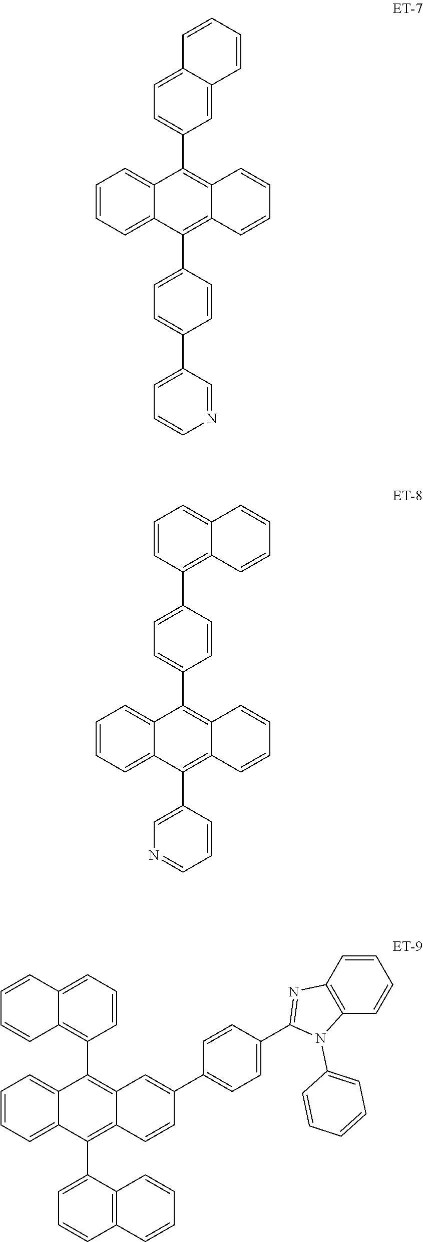 [SMILES: c1cncc(-c2ccc(-c3c4ccccc4c(-c4ccc5ccccc5c4)c4ccccc34)cc2)c1 ; c1cncc(-c2c3ccccc3c(-c3ccc(-c4cccc5ccccc45)cc3)c3ccccc23)c1 ; c1ccc(-n2c(-c3ccc(-c4ccc5c(-c6cccc7ccccc67)c6ccccc6c(-c6cccc7ccccc67)c5c4)cc3)nc3ccccc32)cc1]

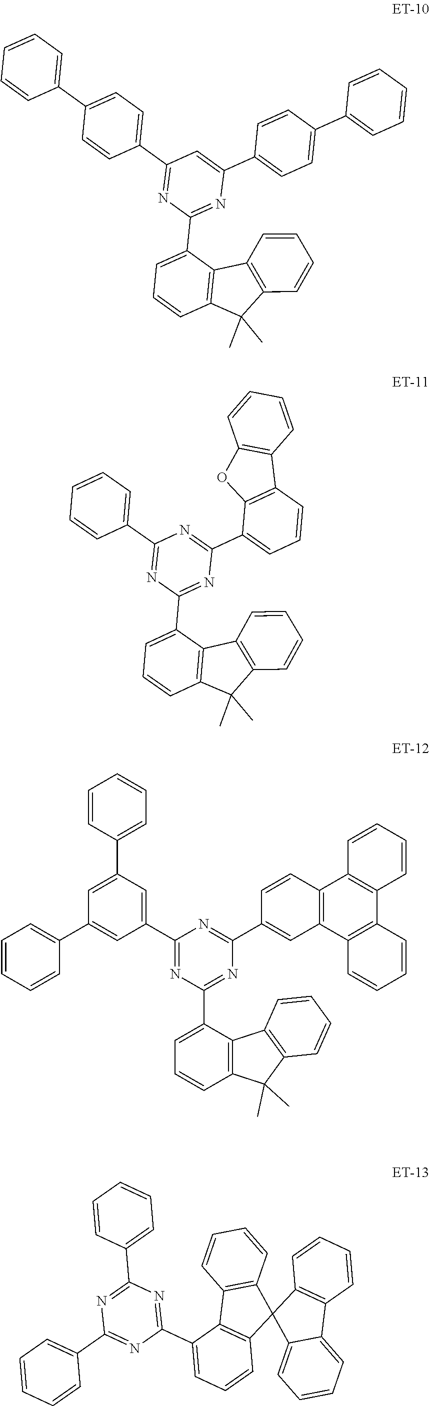 [SMILES: CC1(C)c2ccccc2-c2c(-c3nc(-c4ccc(-c5ccccc5)cc4)cc(-c4ccc(-c5ccccc5)cc4)n3)cccc21 ; CC1(C)c2ccccc2-c2c(-c3nc(-c4ccccc4)nc(-c4cccc5c4oc4ccccc45)n3)cccc21 ; CC1(C)c2ccccc2-c2c(-c3nc(-c4cc(-c5ccccc5)cc(-c5ccccc5)c4)nc(-c4ccc5c6ccccc6c6ccccc6c5c4)n3)cccc21 ; c1ccc(-c2nc(-c3ccccc3)nc(-c3cccc4c3-c3ccccc3C43c4ccccc4-c4ccccc43)n2)cc1]

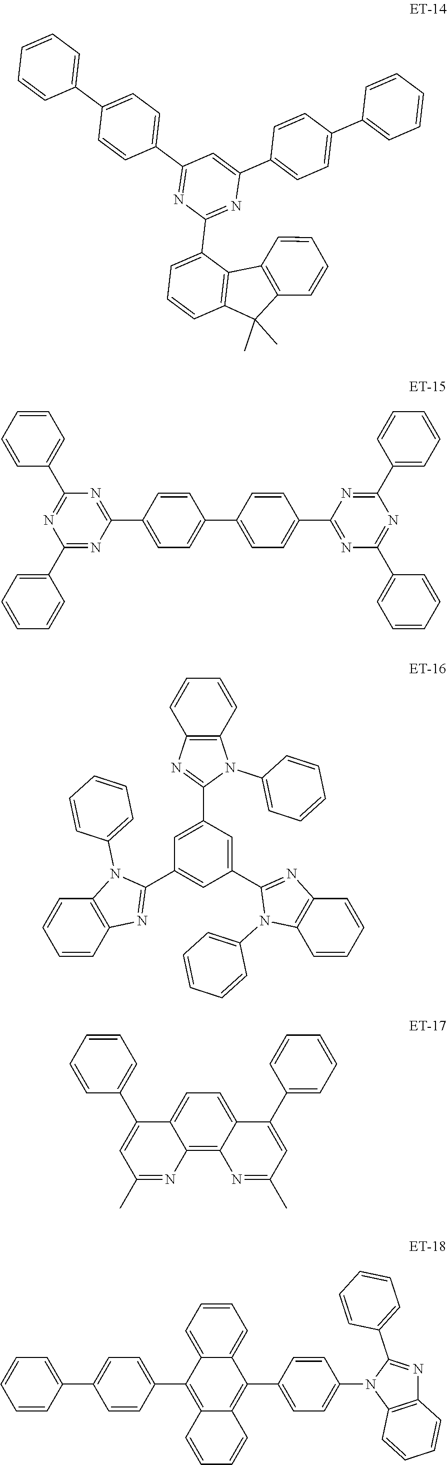 [SMILES: CC1(C)c2ccccc2-c2c(-c3nc(-c4ccc(-c5ccccc5)cc4)cc(-c4ccc(-c5ccccc5)cc4)n3)cccc21 ; c1ccc(-c2nc(-c3ccccc3)nc(-c3ccc(-c4ccc(-c5nc(-c6ccccc6)nc(-c6ccccc6)n5)cc4)cc3)n2)cc1 ; c1ccc(-n2c(-c3cc(-c4nc5ccccc5n4-c4ccccc4)cc(-c4nc5ccccc5n4-c4ccccc4)c3)nc3ccccc32)cc1 ; Cc1cc(-c2ccccc2)c2ccc3c(-c4ccccc4)cc(C)nc3c2n1 ; c1ccc(-c2ccc(-c3c4ccccc4c(-c4ccc(-n5c(-c6ccccc6)nc6ccccc65)cc4)c4ccccc34)cc2)cc1]

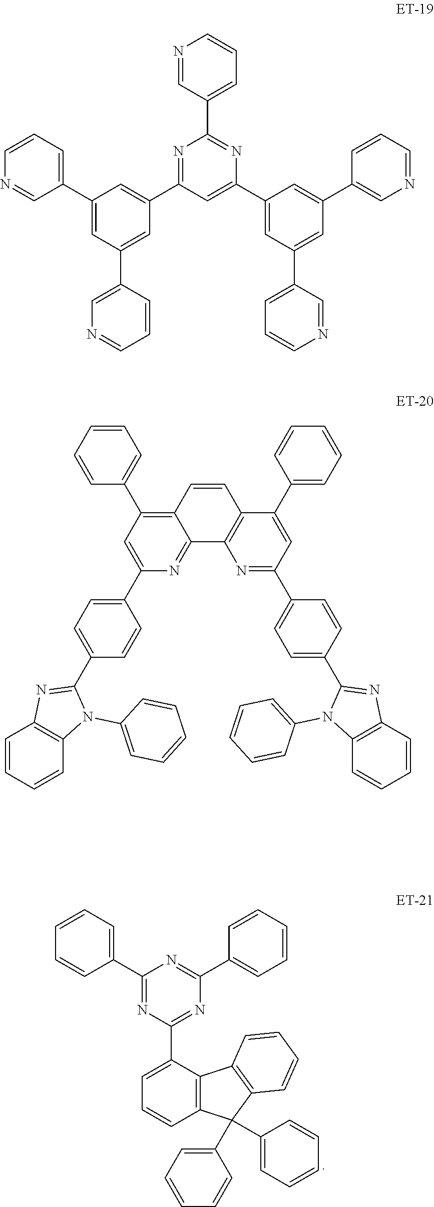 [SMILES: c1cncc(-c2cc(-c3cccnc3)cc(-c3cc(-c4cc(-c5cccnc5)cc(-c5cccnc5)c4)nc(-c4cccnc4)n3)c2)c1 ; c1ccc(-c2cc(-c3ccc(-c4nc5ccccc5n4-c4ccccc4)cc3)nc3c2ccc2c(-c4ccccc4)cc(-c4ccc(-c5nc6ccccc6n5-c5ccccc5)cc4)nc23)cc1 ; c1ccc(-c2nc(-c3ccccc3)nc(-c3cccc4c3-c3ccccc3C4(c3ccccc3)c3ccccc3)n2)cc1]

In the present application, the cathode 200 may comprise a cathode material, which is a low work function material contributing to injection of electrons into the functional layer. Specific examples of the cathode material include, but are not limited to, metals such as magnesium, calcium, sodium, potassium, titanium, indium, yttrium, lithium, gadolinium, aluminum, silver, tin, and lead, or alloys thereof; or multilayer materials such as LiF/Al, Liq/Al, LiO₂/Al, LiF/Ca, LiF/Al and BaF₂/Ca. Preferably, a metal electrode comprising magnesium and silver as the cathode is included.

Alternatively, as shown in FIG. 1, a hole injection layer 310 may be further provided between the anode 100 and the first hole transport layer 321 to enhance the ability to inject holes into the first hole transport layer 321. The hole injection layer 310 may choose to use a benzidine derivative, a starburst arylamine-based compound, a phthalocyanine derivative or other materials. It is not particularly limited in the present application. For example, the hole injection layer 310 contains a compound selected from the group consisting of the following compounds:

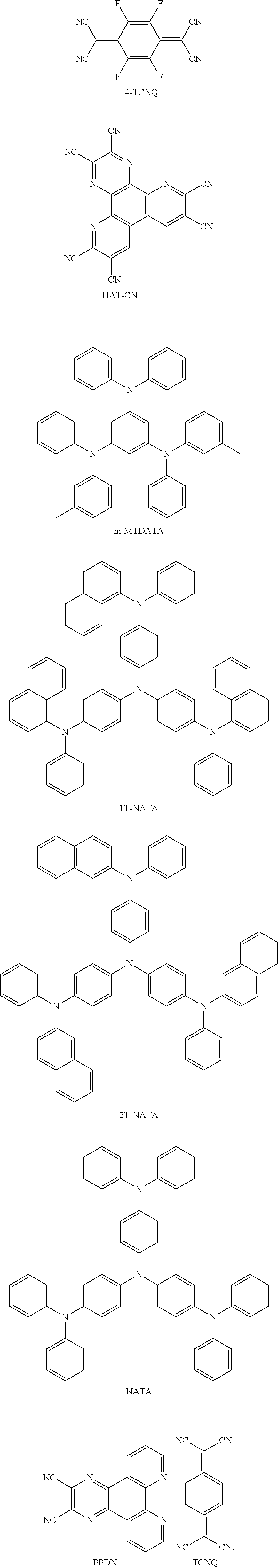

In one specific embodiment of the present application, the hole injection layer 310 is F4-TCNQ.

Alternatively, as shown in FIG. 1, an electron injection layer 350 may be further provided between the cathode 200 and the electron transport layer 340 to enhance the ability to inject electrons into the electron transport layer 340. The electron injection layer 350 may include an inorganic material such as an alkali metal sulfide and an alkali metal halide or may comprise a complex of an alkali metal and an organic compound. For example, the electron injection layer 350 includes LiQ.

According to another embodiment, the electronic element is a photoelectric conversion device. As shown in FIG. 3, the photoelectric conversion device may include an anode 100 and a cathode 200 disposed opposite each other, and a functional layer 300 disposed between the anode 100 and the cathode 200; the functional layer 300 comprises an organic compound provided in the present application.

According to one specific embodiment, as shown in FIG. 3, the photoelectric conversion device includes an anode 100, a bole transport layer 320, a photoelectric conversion layer 360, an electron transport layer 340, and a cathode 200 disposed that are stacked sequentially. Alternatively, the hole transport layer 320 comprises an organic compound of the present application.

Alternatively, the photoelectric conversion device may be a solar cell, in particular may be an organic thin film solar cell. By way of example, in one embodiment of the present application, the solar cell comprises an anode, a bole transport layer, a photoelectric conversion layer, an electron transport layer, and a cathode that are stacked sequentially, wherein the hole transport layer comprises an organic compound of the present application.

In a third aspect, the present application provides an electronic apparatus, comprising the electronic element described in the second aspect.

According to one embodiment, as shown in FIG. 2, the electronic apparatus is a first electronic apparatus 400 comprising the above-described organic electroluminescent device. The first electronic apparatus 400 may be, for example, a display apparatus, a lighting apparatus, an optical communication apparatus, or other type of electronic apparatus, examples of which may include, for example but be not limited to, computer screens, mobile phone screens, televisions, electronic paper, emergency lamps, optical modules, etc.

According to another embodiment, as shown in FIG. 4, the electronic apparatus is a second electronic apparatus 500 comprising the above-described photoelectric conversion device. The second electronic apparatus 500 may be, for example, a solar power supply equipment, a light detector, a fingerprint identification equipment, an optical module, a CCD camera, or other type of electronic apparatus.

The synthesis method of the organic compounds of the present application will be demonstrated in detail with the following synthetic examples, but the present application is not limited in any way by this.

Compounds for which synthesis methods are not mentioned in the present application are all commercially available as raw material products.

### SYNTHETIC EXAMPLE

### 1. Synthesis of IM e-1

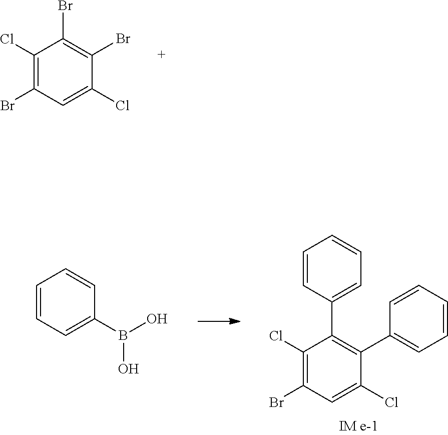 [SMILES: Clc1cc(Br)c(Cl)c(Br)c1Br ; OB(O)c1ccccc1 ; Clc1cc(Br)c(Cl)c(-c2ccccc2)c1-c1ccccc1]

A three-necked flask equipped with a mechanical stirrer, thermometer, and spherical reflux condenser was purged with nitrogen gas (0.100 L/min) for 15 min. Raw materials of 1,3,4-tribromo-2,5-dichlorobenzene (50 g, 130.3 mmol), phenylboronic acid (35 g, 286.7 mmol), tetrakis(triphenylphosphine) palladium (0.75 g, 0.65 mmol), and potassium carbonate (71.9 g, 520.2 mmol) were sequentially added, followed by the addition of a mixed solvent of toluene (400 mL), ethanol (200 mL), and water (100 mL). The mixture was stirred and heated to 78° C. to 80° C. for 12 h of reaction. After the reaction was completed, it was cooled to room temperature. The reaction solution was washed with water, and then the organic phase was separated and dried over anhydrous magnesium sulfate. The mixture was filtered, and the filtrate was distillated under reduced pressure to remove the solvent, thereby to afford a crude product. The crude product was purified via silica gel column chromatography using a dichloromethane/n-heptane system, to afford IM e-1 (20.7 g, yield: 42%).

IM e-2 listed in Table 1 was synthesized by the same method as IM e-1, except that Raw material 1 was used instead of 1,3,4-tribromo-2,5-dichlorobenzene, wherein the main raw materials used, the intermediates synthesized, and their yields are shown in Table 1:

TABLE 1

| Raw material 1 | IM e-2 | Yield/% |
|---|---|---|
| 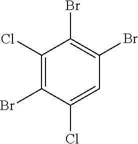 [SMILES: Clc1cc(Br)c(Br)c(Cl)c1Br] | 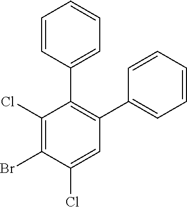 [SMILES: Clc1cc(-c2ccccc2)c(-c2ccccc2)c(Cl)c1Br] | 39 |

### 2. Synthesis of IM f-1

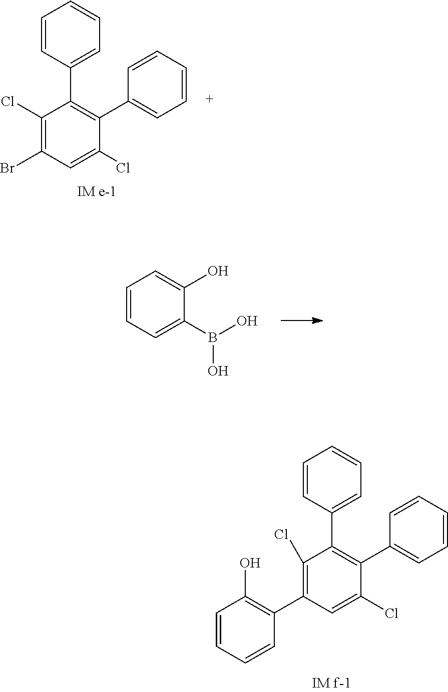 [SMILES: Clc1cc(Br)c(Cl)c(-c2ccccc2)c1-c1ccccc1 ; OB(O)c1ccccc1O ; Oc1ccccc1-c1cc(Cl)c(-c2ccccc2)c(-c2ccccc2)c1Cl]

A three-necked flask equipped with a mechanical stirrer, thermometer, and spherical reflux condenser was purged with nitrogen gas (0.100 L/min) for 15 min. IM e-1 (20 g, 52.9 mmol), o-hydroxyphenylboronic acid (8 g, 58.0 mmol), tetrakis(triphenylphosphine) palladium (0.61 g, 0.53 mmol), and potassium carbonate (14.6 g, 105.8 mmol) were sequentially added, followed by the addition of a mixed solvent of toluene (160 mL), ethanol (80 mL), and water (40 mL). The mixture was stirred and heated to 78° C. to 80° C. for 8 h of reaction. After the reaction was completed, it was cooled to room temperature. The reaction solution was washed with water, and then the organic phase was separated and dried over anhydrous magnesium sulfate. The mixture was filtered, and the filtrate was distillated under reduced pressure to remove the solvent, thereby to afford a crude product. The crude product was purified via silica gel column chromatography using a dichloromethane/n-heptane system, to afford IM f-1 (17 g, yield: 82%).

IM f-x listed in Table 2 was synthesized by the same method as IM f-1, except that Raw material 2 was used instead of IM e-1 and Raw material 3 was used instead of o-bydroxyphenylboronic acid, wherein the main raw materials used, the intermediates synthesized and their yields are shown in Table 2:

TABLE 2

| Raw material 2 | Raw material 3 | IM f-x | Yield/% |
|---|---|---|---|
| 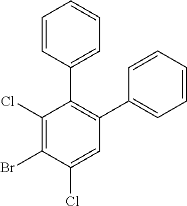 [SMILES: Clc1cc(-c2ccccc2)c(-c2ccccc2)c(Cl)c1Br] | 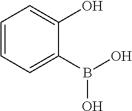 | 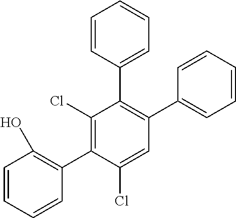 [SMILES: Oc1ccccc1-c1c(Cl)cc(-c2ccccc2)c(-c2ccccc2)c1Cl] | 73 |
| 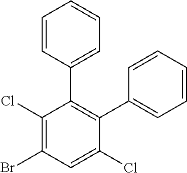 [SMILES: Clc1cc(Br)c(Cl)c(-c2ccccc2)c1-c1ccccc1] | 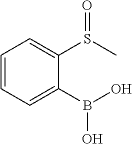 | 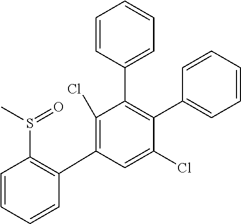 [SMILES: CS(=O)c1ccccc1-c1cc(Cl)c(-c2ccccc2)c(-c2ccccc2)c1Cl] | 70 |
| 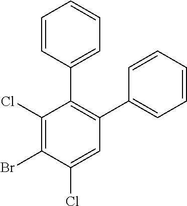 [SMILES: Clc1cc(-c2ccccc2)c(-c2ccccc2)c(Cl)c1Br] | 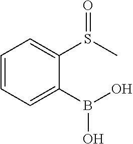 | 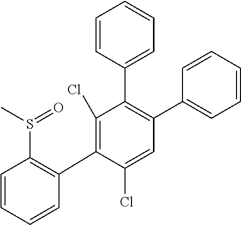 [SMILES: CS(=O)c1ccccc1-c1c(Cl)cc(-c2ccccc2)c(-c2ccccc2)c1Cl] | 69 |

### 3. Synthesis of IM G-1

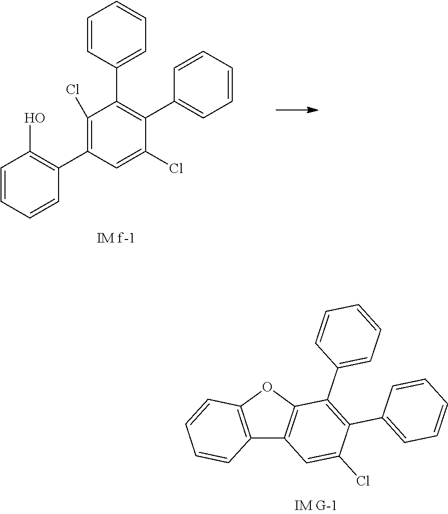 [SMILES: Oc1ccccc1-c1cc(Cl)c(-c2ccccc2)c(-c2ccccc2)c1Cl ; Clc1cc2c(oc3ccccc32)c(-c2ccccc2)c1-c1ccccc1]

A three-necked flask equipped with a mechanical stirrer, thermometer, and spherical reflux condenser was purged with nitrogen gas (0.200 L/min) for 15 min, and IM f-1 (17 g, 43.4 mmol), potassium carbonate (12 g, 86.8 mmol), and acetonitrile (150 mL) were added sequentially, the reaction was refluxed for 5 h. At the end of the reaction, the reaction was cooled to room temperature. After the organic phase was extracted with dichloromethane and water, it was dried over anhydrous magnesium sulfate. After filtering, the filtrate was distillated under reduced pressure to remove the solvent to afford a crude product. The crude product was purified via silica gel column chromatography using a dichloromethane/n-heptane system, to afford IM G-1 (13.6 g, yield: 88%).

IM G-2 listed in Table 3 was synthesized by the same method as IM G-1, except that IM f-2 was used instead of IM f-1, wherein the main raw materials used, the intermediates synthesized, and their yields are shown in Table 3:

TABLE 3

| IM f-2 | IM G-2 | Yield/% |
|---|---|---|
| 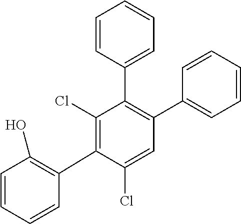 [SMILES: Oc1ccccc1-c1c(Cl)cc(-c2ccccc2)c(-c2ccccc2)c1Cl] | 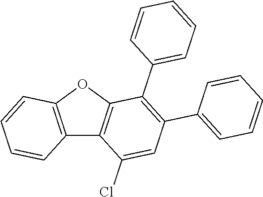 [SMILES: Clc1cc(-c2ccccc2)c(-c2ccccc2)c2oc3ccccc3c12] | 43 |

### 4. Synthesis of IM H-1

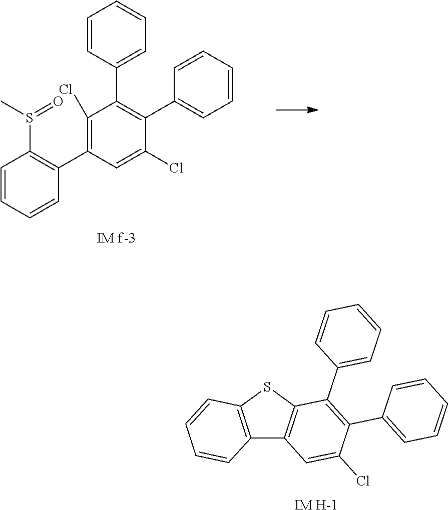

A three-necked flask equipped with a mechanical stirrer, thermometer, and spherical reflux condenser was purged with nitrogen gas (0.100 L/min) for 15 min, and IM f-3 (15 g, 34.3 mmol) and sulfuric acid (50 mL) were added sequentially, and the reaction was refluxed for 6 h. At the end of the reaction, 500 mL of 1 mol/L NaOH solution was added and stirred for 1 h, and the resulting solid was filtered and recrystallized with toluene to afford IM H-1 (11 g, yield: 86%).

IM H-2 listed in Table 4 was synthesized by the same method as IM H-1, except that IM f-4 was used instead of IM f-3, wherein the main raw materials used, the intermediates synthesized, and their yields are shown in Table 4:

TABLE 4

| IM f-4 | IM H-2 | Yield/% |
|---|---|---|
| 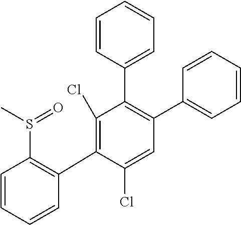 [SMILES: CS(=O)c1ccccc1-c1c(Cl)cc(-c2ccccc2)c(-c2ccccc2)c1Cl] | 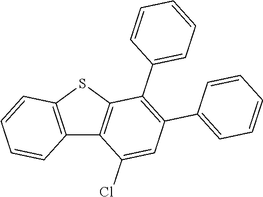 [SMILES: Clc1cc(-c2ccccc2)c(-c2ccccc2)c2sc3ccccc3c12] | 46 |

### 5. Synthesis of IM i-1

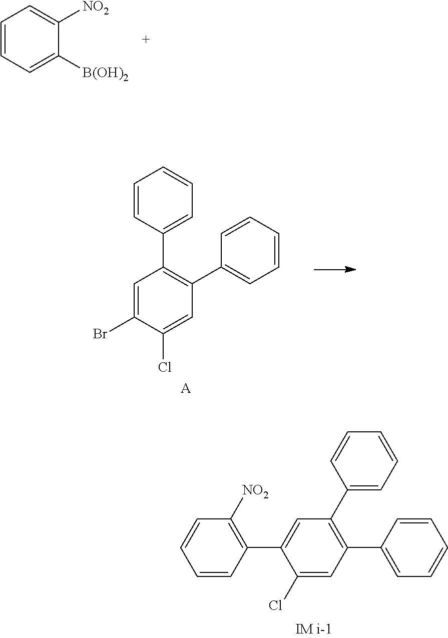

A three-necked flask equipped with a mechanical stirrer, thermometer, and spherical reflux condenser was purged with nitrogen gas (0.100 L/min) for 15 min, and the raw materials of 2-nitrophenylboronic acid (50.0 g, 277.82 mmol), Reactant A (95.47 g, 277.82 mmol), bis(triphenylphosphine) palladium dichloride (0.975 g, 1.4 mmol) and potassium carbonate (95.99 g, 694.56 mmol) were added sequentially, followed by the addition of a solvent mixture of ethylene glycol dimethyl ether (400 mL) and water (100 mL). The mixture was stirred and heated to 78° C. to 80° C. for 15 h of reaction. After the reaction was completed, it was cooled to room temperature. The reaction solution was washed with water, and then the organic phase was separated and dried over anhydrous magnesium sulfate. The mixture was filtered, and the filtrate was distillated under reduced pressure to remove the solvent, thereby to afford a crude product. The crude product was purified via silica gel column chromatography using a dichloromethane/n-heptane system, to afford IM i-1 (72.03 g, yield: 65%) as a withe solid.

IM j-1 listed in Table 5 was synthesized by the same method as IM i-1, except that Raw material 4 was used instead of Reactant A, wherein the main raw materials used, the intermediates synthesized, and their yields are shown in Table 5:

TABLE 5

| Raw material 4 | IM j-1 | Yield/% |
|---|---|---|
| 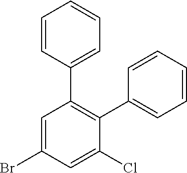 [SMILES: Clc1cc(Br)cc(-c2ccccc2)c1-c1ccccc1] | 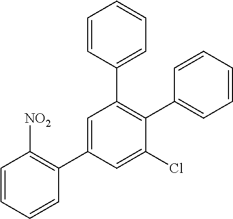 [SMILES: O=[N+]([O-])c1ccccc1-c1cc(Cl)c(-c2ccccc2)c(-c2ccccc2)c1] | 60 |

### 6. Synthesis of IM i-2

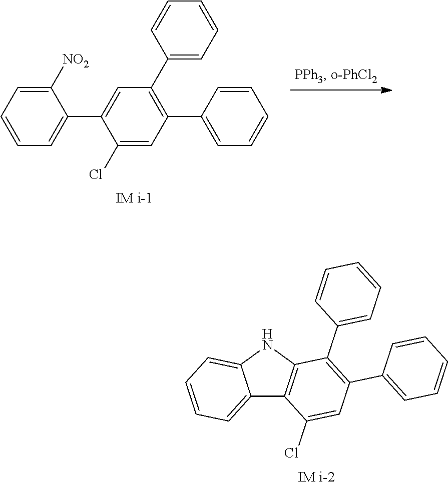 [SMILES: O=[N+]([O-])c1ccccc1-c1cc(-c2ccccc2)c(-c2ccccc2)cc1Cl ; Clc1cc(-c2ccccc2)c(-c2ccccc2)c2[nH]c3ccccc3c12]

To a flask were added IM i-1 (72 g, 186.6 mmol), triphenylphosphine (122.35 g, 466.5 mmol) and o-dichlorobenzene (720 mL). The mixture was heated to 175° C. under nitrogen protection, stirred for 18 h and then cooled to room temperature. The reaction solution was washed with water and separated, the organic phase was washed with water and then dried over anhydrous magnesium sulfate, and solvents were removed at high temperature and reduced pressure to afford a crude product. The crude product was purified via silica gel column chromatography using an ethyl acetate/n-heptane system, to afford IM i-2 (56.78 g, yield: 88%) as a white solid.

IM j-2 listed in Table 6 was synthesized by the same method as IM i-2, except that IM j-1 was used instead of IM i-1, wherein the main raw materials used, the intermediates synthesized, and their yields are shown in Table 6:

TABLE 6

| IM j-1 | IM j-2 | Yield/% |
|---|---|---|
| 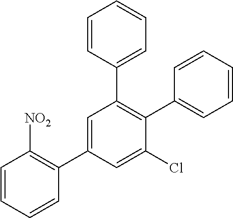 [SMILES: O=[N+]([O-])c1ccccc1-c1cc(Cl)c(-c2ccccc2)c(-c2ccccc2)c1] | 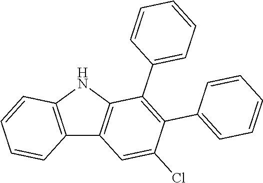 [SMILES: Clc1cc2c([nH]c3ccccc32)c(-c2ccccc2)c1-c1ccccc1] | 83 |

### 7. Synthesis of IM I-1

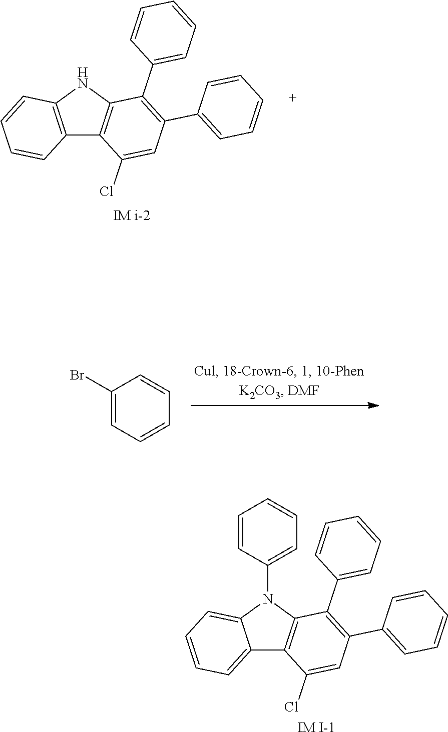

To a flask were added IM i-2 (50 g, 141.3 mmol), bromobenzene (22.18 g, 141.3 mmol), cuprous iodide (2.69 g, 14.1 mmol), potassium carbonate (48.8 g, 353.3 mmol), 1,10-phenanthroline (5.6 g, 28.3 mmol), 18-crown-6-ether (3.7 g, 14.1 mmol) and dimethylformamide (50 mL). The mixture heated to 130° C. under nitrogen protection and stirred for 12 h. After cooling to room temperature, the reaction solution was added with dichloromethane (500 mL) and water and separated, the organic phase was washed with water and then dried over anhydrous magnesium sulfate, and solvents were removed under reduced pressure to afford a crude product. After the crude product was purified by silica gel column chromatography using dichloromethane/n-heptane system, it was purified by recrystallization using toluene/n-heptane system to obtain IM. I-1 (30.37 g, yield: 50%) as a white solid.

IM J-1 listed in Table 7 was synthesized by the same method as IM I-1, except that IM j-2 was used instead of IM i-2, wherein the main raw materials used, the intermediates synthesized, and their yields are shown in Table 7:

TABLE 7

| IM j-2 | IM J-1 | Yield/% |
|---|---|---|
| 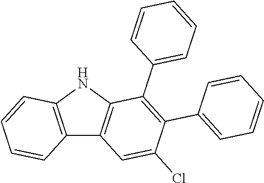 [SMILES: Clc1cc2c([nH]c3ccccc32)c(-c2ccccc2)c1-c1ccccc1] | 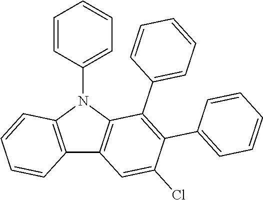 [SMILES: Clc1cc2c3ccccc3n(-c3ccccc3)c2c(-c2ccccc2)c1-c1ccccc1] | 83 |

### 8. Synthesis of Compound A-1-3

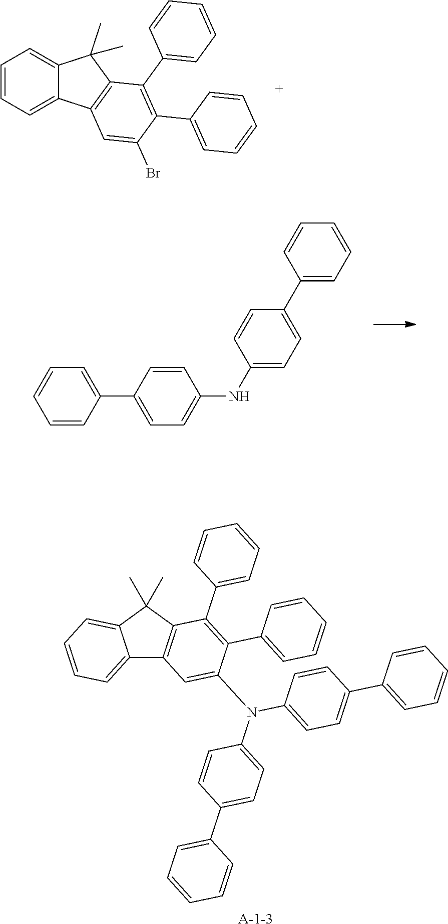

A three-necked flask equipped with a mechanical stirrer, thermometer, and spherical reflux condenser was purged with nitrogen gas (0.200 L/min) for 15 min, and 3-bromo-9,9-dimethyl-1,2-diphenyl-9H-fluorene (5 g, 11.8 mmol), bis(4-biphenylyl)amine (3.8 g, 11.8 mmol), tris(dibenzylideneacetone) dipalladium (0.11 g, 0.12 mmol), 2-bis-dicyclohexylphosphino-2′,6′-dimethoxybiphenyl (0.1 g, 0.24 mmol), sodium tert-butoxide (1.7 g, 17.7 mmol), and toluene (50 mL) were added sequentially, and the reaction was refluxed at 105° C. to 110° C. for 4 h, and at the end of the reaction, it was cooled to room temperature. After the organic phase was washed with water, it was dried over anhydrous magnesium sulfate. After filtering, the filtrate was distillated under reduced pressure to remove the solvent to afford a crude product. The crude product was purified via silica gel column chromatography using a dichloromethane/n-heptane system, to afford compound A-1-3 (5.4 g, yield: 69%) as a white solid. Mass spectrometry (m/z)=666.31 [M+H]⁺.

The compounds listed in Table 8 below was synthesized by the same method as Compound A-1-3, except that Raw material 5 was used instead of 3-bromo-9,9-dimethyl-1,2-diphenyl-9H-fluorene, and Raw material 6 was used instead of bis(4-biphenylyl)amine, wherein the main raw materials used, the compounds synthesized and their yields, and mass spectrometry are shown in Table 8:

TABLE 8

| | | | | Mass |
|---|---|---|---|---|
| | | | | spectrometry |
| Raw | Raw | Compound | Yield/ | (m/z) |
| material 5 | material 6 | structure | % | [M + H]⁺ |
| 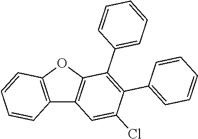 [SMILES: Clc1cc2c(oc3ccccc32)c(-c2ccccc2)c1-c1ccccc1] IM G-1 | 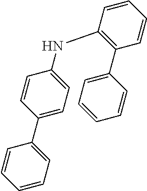 [SMILES: c1ccc(-c2ccc(Nc3ccccc3-c3ccccc3)cc2)cc1] | 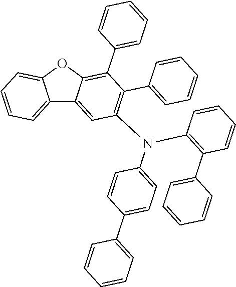 [SMILES: c1ccc(-c2ccc(N(c3ccccc3-c3ccccc3)c3cc4c(oc5ccccc54)c(-c4ccccc4)c3-c3ccccc3)cc2)cc1] | 55 | 640.26 |
| | | B-1-2 | | |
| 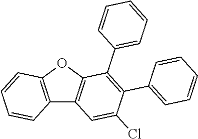 [SMILES: Clc1cc2c(oc3ccccc32)c(-c2ccccc2)c1-c1ccccc1] IM G-1 | 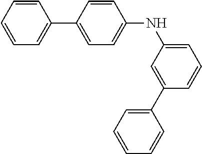 [SMILES: c1ccc(-c2ccc(Nc3cccc(-c4ccccc4)c3)cc2)cc1] | 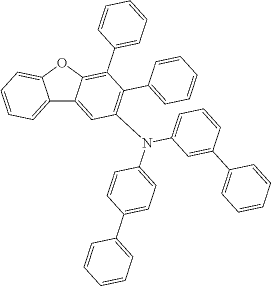 [SMILES: c1ccc(-c2ccc(N(c3cccc(-c4ccccc4)c3)c3cc4c(oc5ccccc54)c(-c4ccccc4)c3-c3ccccc3)cc2)cc1] | 63 | 640.26 |
| | | B-1-5 | | |
| 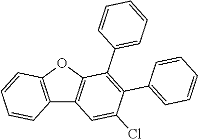 [SMILES: Clc1cc2c(oc3ccccc32)c(-c2ccccc2)c1-c1ccccc1] IM G-1 | 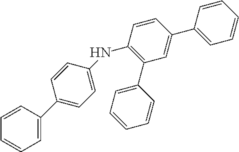 [SMILES: c1ccc(-c2ccc(Nc3ccc(-c4ccccc4)cc3-c3ccccc3)cc2)cc1] | 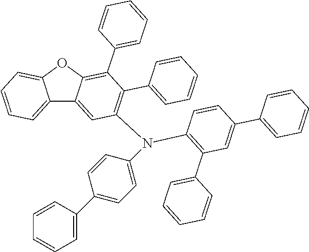 [SMILES: c1ccc(-c2ccc(N(c3ccc(-c4ccccc4)cc3-c3ccccc3)c3cc4c(oc5ccccc54)c(-c4ccccc4)c3-c3ccccc3)cc2)cc1] | 64 | 716.29 |
| | | B-1-6 | | |
| 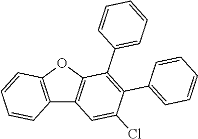 [SMILES: Clc1cc2c(oc3ccccc32)c(-c2ccccc2)c1-c1ccccc1] IM G-1 | 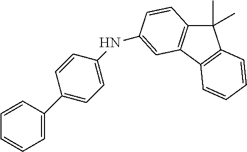 [SMILES: CC1(C)c2ccccc2-c2cc(Nc3ccc(-c4ccccc4)cc3)ccc21] | 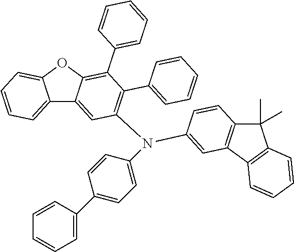 [SMILES: CC1(C)c2ccccc2-c2cc(N(c3ccc(-c4ccccc4)cc3)c3cc4c(oc5ccccc54)c(-c4ccccc4)c3-c3ccccc3)ccc21] | 60 | 680.29 |
| | | B-1-19 | | |
| 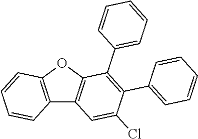 [SMILES: Clc1cc2c(oc3ccccc32)c(-c2ccccc2)c1-c1ccccc1] IM G-1 | 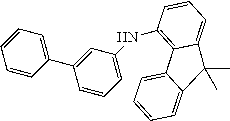 [SMILES: CC1(C)c2ccccc2-c2c(Nc3cccc(-c4ccccc4)c3)cccc21] | 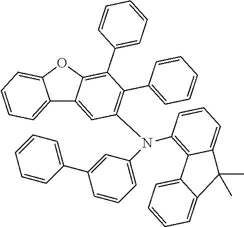 [SMILES: CC1(C)c2ccccc2-c2c(N(c3cccc(-c4ccccc4)c3)c3cc4c(oc5ccccc54)c(-c4ccccc4)c3-c3ccccc3)cccc21] | 57 | 680.29 |
| | | B-1-20 | | |
| 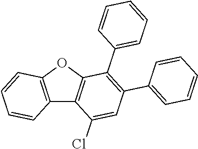 [SMILES: Clc1cc(-c2ccccc2)c(-c2ccccc2)c2oc3ccccc3c12] IM G-2 | 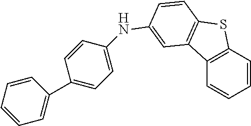 [SMILES: c1ccc(-c2ccc(Nc3ccc4sc5ccccc5c4c3)cc2)cc1] | 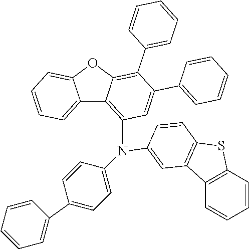 [SMILES: c1ccc(-c2ccc(N(c3ccc4sc5ccccc5c4c3)c3cc(-c4ccccc4)c(-c4ccccc4)c4oc5ccccc5c34)cc2)cc1] | 58 | 670.22 |
| | | B-1-29 | | |
| 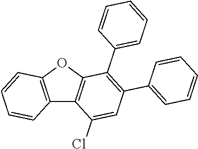 [SMILES: Clc1cc(-c2ccccc2)c(-c2ccccc2)c2oc3ccccc3c12] IM G-2 | 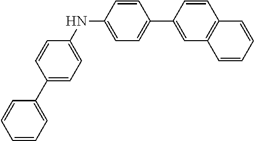 [SMILES: c1ccc(-c2ccc(Nc3ccc(-c4ccc5ccccc5c4)cc3)cc2)cc1] | 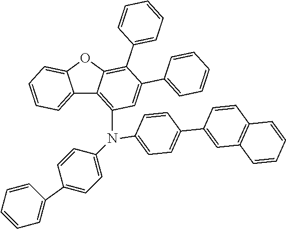 [SMILES: c1ccc(-c2ccc(N(c3ccc(-c4ccc5ccccc5c4)cc3)c3cc(-c4ccccc4)c(-c4ccccc4)c4oc5ccccc5c34)cc2)cc1] | 65 | 690.28 |
| | | B-1-36 | | |
| 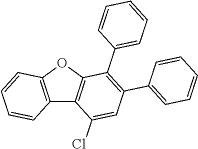 [SMILES: Clc1cc(-c2ccccc2)c(-c2ccccc2)c2oc3ccccc3c12] IM G-2 | 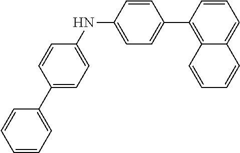 [SMILES: c1ccc(-c2ccc(Nc3ccc(-c4cccc5ccccc45)cc3)cc2)cc1] | 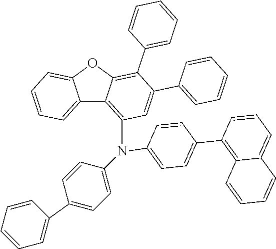 [SMILES: c1ccc(-c2ccc(N(c3ccc(-c4cccc5ccccc45)cc3)c3cc(-c4ccccc4)c(-c4ccccc4)c4oc5ccccc5c34)cc2)cc1] | 60 | 690.28 |
| | | B-1-37 | | |
| 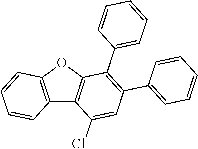 [SMILES: Clc1cc(-c2ccccc2)c(-c2ccccc2)c2oc3ccccc3c12] IM G-2 | 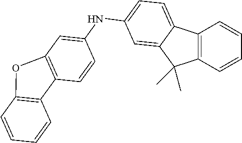 [SMILES: CC1(C)c2ccccc2-c2ccc(Nc3ccc4c(c3)oc3ccccc34)cc21] | 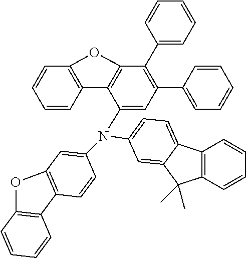 [SMILES: CC1(C)c2ccccc2-c2ccc(N(c3ccc4c(c3)oc3ccccc34)c3cc(-c4ccccc4)c(-c4ccccc4)c4oc5ccccc5c34)cc21] | 62 | 694.27 |
| | | B-1-43 | | |
| 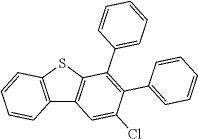 [SMILES: Clc1cc2c(sc3ccccc32)c(-c2ccccc2)c1-c1ccccc1] IM H-1 | 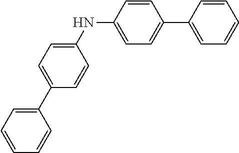 [SMILES: c1ccc(-c2ccc(Nc3ccc(-c4ccccc4)cc3)cc2)cc1] | 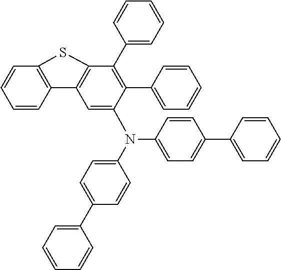 [SMILES: c1ccc(-c2ccc(N(c3ccc(-c4ccccc4)cc3)c3cc4c(sc5ccccc54)c(-c4ccccc4)c3-c3ccccc3)cc2)cc1] | 65 | 656.24 |
| | | C-1-1 | | |
| 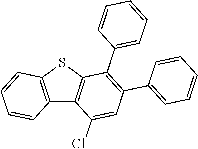 [SMILES: Clc1cc(-c2ccccc2)c(-c2ccccc2)c2sc3ccccc3c12] IM H-2 | 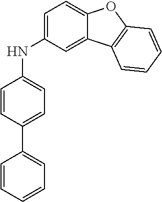 [SMILES: c1ccc(-c2ccc(Nc3ccc4oc5ccccc5c4c3)cc2)cc1] | 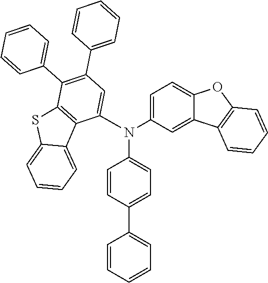 [SMILES: c1ccc(-c2ccc(N(c3ccc4oc5ccccc5c4c3)c3cc(-c4ccccc4)c(-c4ccccc4)c4sc5ccccc5c34)cc2)cc1] | 64 | 670.22 |
| | | C-1-7 | | |
| 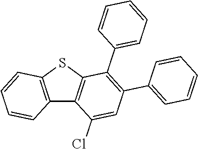 [SMILES: Clc1cc(-c2ccccc2)c(-c2ccccc2)c2sc3ccccc3c12] IM H-2 | 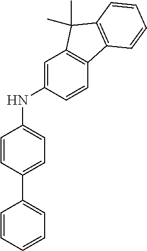 [SMILES: CC1(C)c2ccccc2-c2ccc(Nc3ccc(-c4ccccc4)cc3)cc21] | 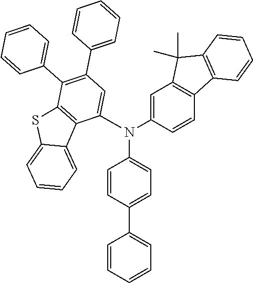 [SMILES: CC1(C)c2ccccc2-c2ccc(N(c3ccc(-c4ccccc4)cc3)c3cc(-c4ccccc4)c(-c4ccccc4)c4sc5ccccc5c34)cc21] | 57 | 696.27 |
| | | C-1-10 | | |
| 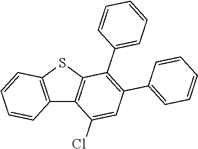 [SMILES: Clc1cc(-c2ccccc2)c(-c2ccccc2)c2sc3ccccc3c12] IM H-2 | 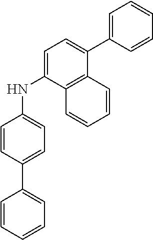 [SMILES: c1ccc(-c2ccc(Nc3ccc(-c4ccccc4)c4ccccc34)cc2)cc1] | 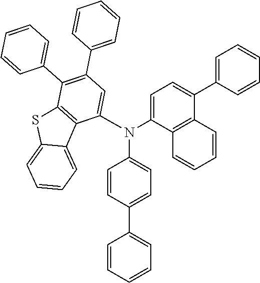 [SMILES: c1ccc(-c2ccc(N(c3ccc(-c4ccccc4)c4ccccc34)c3cc(-c4ccccc4)c(-c4ccccc4)c4sc5ccccc5c34)cc2)cc1] | 60 | 706.25 |
| | | C-1-14 | | |
| 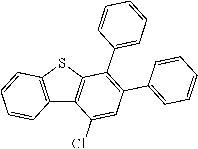 [SMILES: Clc1cc(-c2ccccc2)c(-c2ccccc2)c2sc3ccccc3c12] IM H-2 | 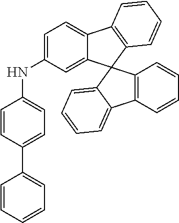 [SMILES: c1ccc(-c2ccc(Nc3ccc4c(c3)C3(c5ccccc5-c5ccccc53)c3ccccc3-4)cc2)cc1] | 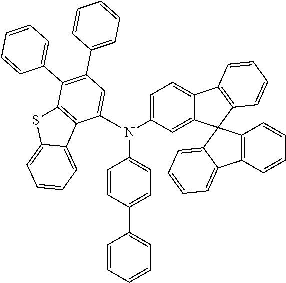 [SMILES: c1ccc(-c2ccc(N(c3ccc4c(c3)C3(c5ccccc5-c5ccccc53)c3ccccc3-4)c3cc(-c4ccccc4)c(-c4ccccc4)c4sc5ccccc5c34)cc2)cc1] | 56 | 818.28 |
| | | C-1-18 | | |
| 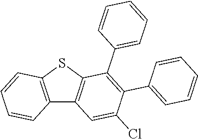 [SMILES: Clc1cc2c(sc3ccccc32)c(-c2ccccc2)c1-c1ccccc1] IM H-1 | 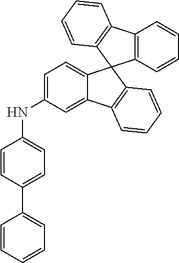 [SMILES: c1ccc(-c2ccc(Nc3ccc4c(c3)-c3ccccc3C43c4ccccc4-c4ccccc43)cc2)cc1] | 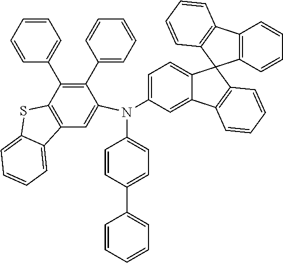 [SMILES: c1ccc(-c2ccc(N(c3ccc4c(c3)-c3ccccc3C43c4ccccc4-c4ccccc43)c3cc4c(sc5ccccc54)c(-c4ccccc4)c3-c3ccccc3)cc2)cc1] | 59 | 818.28 |
| | | C-1-19 | | |
| 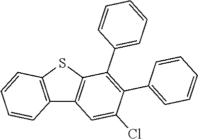 [SMILES: Clc1cc2c(sc3ccccc32)c(-c2ccccc2)c1-c1ccccc1] IM H-1 | 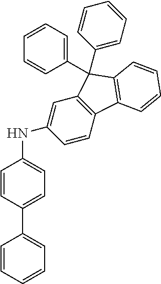 [SMILES: c1ccc(-c2ccc(Nc3ccc4c(c3)C(c3ccccc3)(c3ccccc3)c3ccccc3-4)cc2)cc1] | 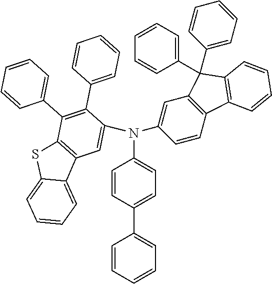 [SMILES: c1ccc(-c2ccc(N(c3ccc4c(c3)C(c3ccccc3)(c3ccccc3)c3ccccc3-4)c3cc4c(sc5ccccc54)c(-c4ccccc4)c3-c3ccccc3)cc2)cc1] | 66 | 820.30 |
| | | C-1-23 | | |
| 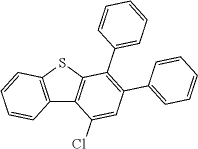 [SMILES: Clc1cc(-c2ccccc2)c(-c2ccccc2)c2sc3ccccc3c12] IM H-2 | 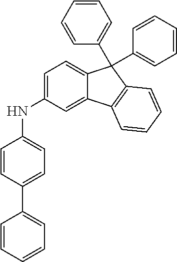 [SMILES: c1ccc(-c2ccc(Nc3ccc4c(c3)-c3ccccc3C4(c3ccccc3)c3ccccc3)cc2)cc1] | 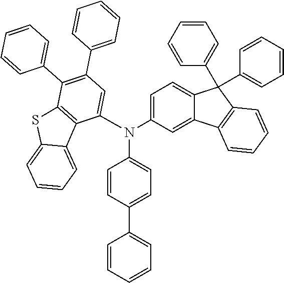 [SMILES: c1ccc(-c2ccc(N(c3ccc4c(c3)-c3ccccc3C4(c3ccccc3)c3ccccc3)c3cc(-c4ccccc4)c(-c4ccccc4)c4sc5ccccc5c34)cc2)cc1] | 63 | 820.30 |
| | | C-1-24 | | |
| 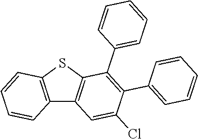 [SMILES: Clc1cc2c(sc3ccccc32)c(-c2ccccc2)c1-c1ccccc1] IM H-1 | 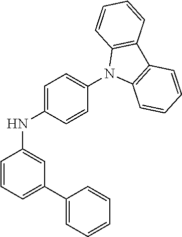 [SMILES: c1ccc(-c2cccc(Nc3ccc(-n4c5ccccc5c5ccccc54)cc3)c2)cc1] | 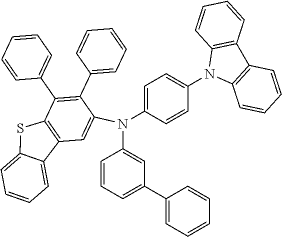 [SMILES: c1ccc(-c2cccc(N(c3ccc(-n4c5ccccc5c5ccccc54)cc3)c3cc4c(sc5ccccc54)c(-c4ccccc4)c3-c3ccccc3)c2)cc1] | 56 | 745.26 |
| | | C-1-32 | | |
| 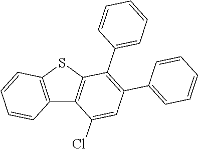 [SMILES: Clc1cc(-c2ccccc2)c(-c2ccccc2)c2sc3ccccc3c12] IM H-2 | 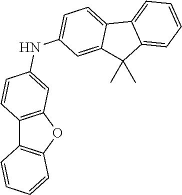 [SMILES: CC1(C)c2ccccc2-c2ccc(Nc3ccc4c(c3)oc3ccccc34)cc21] | 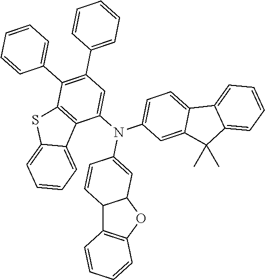 [SMILES: CC1(C)c2ccccc2-c2ccc(N(C3=CC4Oc5ccccc5C4C=C3)c3cc(-c4ccccc4)c(-c4ccccc4)c4sc5ccccc5c34)cc21] | 57 | 710.24 |
| | | C-1-35 | | |
| 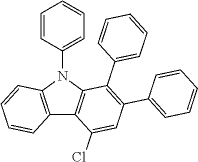 [SMILES: Clc1cc(-c2ccccc2)c(-c2ccccc2)c2c1c1ccccc1n2-c1ccccc1] IM I-1 | 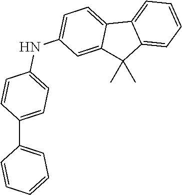 [SMILES: CC1(C)c2ccccc2-c2ccc(Nc3ccc(-c4ccccc4)cc3)cc21] | 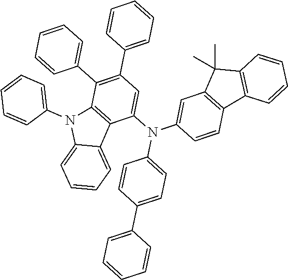 [SMILES: CC1(C)c2ccccc2-c2ccc(N(c3ccc(-c4ccccc4)cc3)c3cc(-c4ccccc4)c(-c4ccccc4)c4c3c3ccccc3n4-c3ccccc3)cc21] | 47 | 755.33 |
| | | D-1-6 | | |
| 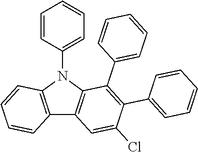 [SMILES: Clc1cc2c3ccccc3n(-c3ccccc3)c2c(-c2ccccc2)c1-c1ccccc1] IM J-2 | 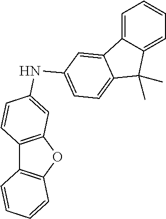 [SMILES: CC1(C)c2ccccc2-c2cc(Nc3ccc4c(c3)oc3ccccc34)ccc21] | 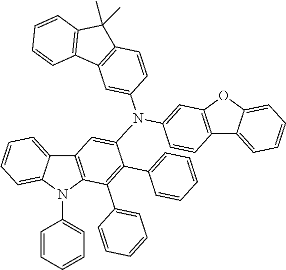 [SMILES: CC1(C)c2ccccc2-c2cc(N(c3ccc4c(c3)oc3ccccc34)c3cc4c5ccccc5n(-c5ccccc5)c4c(-c4ccccc4)c3-c3ccccc3)ccc21] | 46 | 769.31 |
| | | D-1-27 | | |

NMR data for some of the compounds are shown in Table 9 below:

TABLE 9

| Compound | NMR data |
|---|---|
| Compound | ¹H-NMR(400 Hz, CDCl₃): 7.85(d, 1H), 7.55-7.38(m, 21H), |
| A-1-3 | 7.33-7.23(m, 2H), 7.14-7.09(m, 3H), 7.00(d, 2H), 6.48(d, 4H), 1.68(s, 6H). |
| Compound | ¹H-NMR(400 Hz, CDCl₃): 8.21(d, 1H), 7.98(d, 1H), 7.69-7.63(m, 3H), |
| C-1-1 | 7.56-7.38(m, 21H), 7.32-7.28(m, 1H), 7.21-7.15(m, 2H), 6.48(d, 4H). |

### Example 15: Fabrication of Red Organic Electroluminescent Device

The anode is prepared by the following process: the ITO/Ag/ITO substrate with a sequential thickness of 200 Å, 1000 Å, 200 Å was cut into dimensions of 40 mm (length)×40 mm (width)×0.7 mm (thickness), and the substrate was fabricated into an experimental substrate patterned with cathode, and insulating layer through a photolithography process. The experimental substrate was subjected to a surface treatment using UV ozone and O₂:N₂ plasma to enhance the work function of anode (experimental substrate) and to eliminate scum.

F4-TCNQ was vacuum-evaporated on the experimental substrate (anode) to form a hole injection layer with a thickness of 100 Å, and HT-21 was vacuum-evaporated on the hole injection layer to form a first hole transport layer with a thickness of 900 Å.

The compound B-1-2 was vacuum-evaporated on the first hole transport layer to form a second hole transport layer with a thickness of 850 Å.

On the second hole transport layer, CBP and Ir (piq) (acac) were co-evaporated at an evaporation rate ratio of 95%:5%, to form an organic light-emitting layer with a thickness of 360 Å.

On the organic light-emitting layer, ET-21 and LiQ were co-evaporated at an evaporation rate ratio of 1:1 to form an electron transport layer with a thickness of 300 Å. LiQ was vacuum-evaporated on the electron transport layer to form an electron injection layer with a thickness of 15 Å. Then, magnesium (Mg) and silver (Ag) were co-evaporated on the electron injection layer at an evaporation rate ratio of 1:9 to form a cathode with a thickness of 110 Å.

In addition, CP-1 was evaporated on the cathode described above to form a cover layer (CPL) with a thickness of 650 Å, thereby completing the fabrication of organic electroluminescent device.

### Examples 16~35

Organic electroluminescent devices were fabricated using the same method as in Example 15, except that the compounds shown in Table 11 were used instead of the Compound B-1-2 in the formation of the second hole transport layer.

### Comparative Examples 1~3

Organic electroluminescent devices were fabricated using the same method as in Example 15, except that the Compounds A, B, C, and D were used instead of the Compound B-1-2 in the formation of the second hole transport layer.

The structures of the main materials used in the above examples and comparative examples are shown in Table 10 below:

TABLE 10

| | |
|---|---|
| 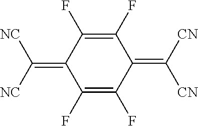 [SMILES: N#CC(C#N)=c1c(F)c(F)c(=C(C#N)C#N)c(F)c1F] | |
| F4-TCNQ | |
| 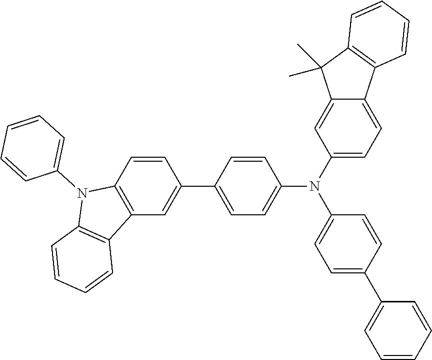 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21] | |
| HT-21 | |
| 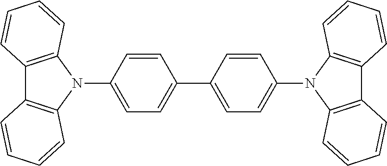 [SMILES: c1ccc2c(c1)c1ccccc1n2-c1ccc(-c2ccc(-n3c4ccccc4c4ccccc43)cc2)cc1] | |
| CBP | |
| 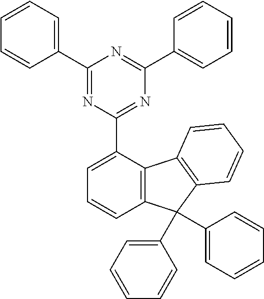 [SMILES: c1ccc(-c2nc(-c3ccccc3)nc(-c3cccc4c3-c3ccccc3C4(c3ccccc3)c3ccccc3)n2)cc1] | |
| ET-21 | |
| 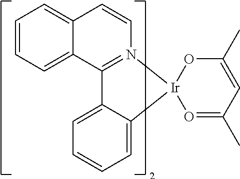 | |
| Ir(piq)₂(acac) | |
| 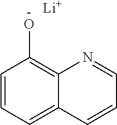 [SMILES: [Li+] ; Oc1cccc2cccnc12] | |
| LiQ | |
| 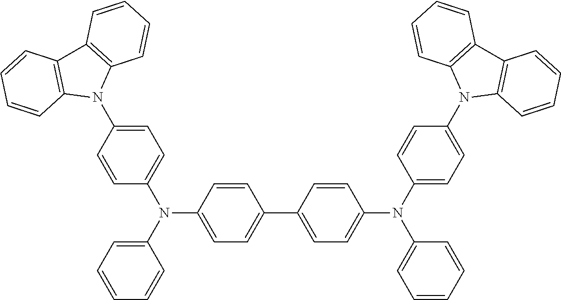 [SMILES: c1ccc(N(c2ccc(-c3ccc(N(c4ccccc4)c4ccc(-n5c6ccccc6c6ccccc65)cc4)cc3)cc2)c2ccc(-n3c4ccccc4c4ccccc43)cc2)cc1] | |
| CP-1 | |
| 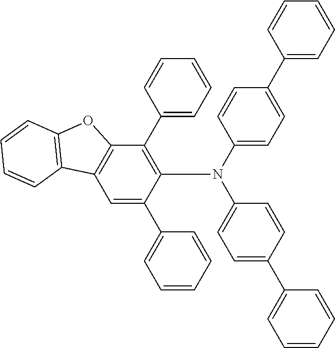 [SMILES: c1ccc(-c2ccc(N(c3ccc(-c4ccccc4)cc3)c3c(-c4ccccc4)cc4c(oc5ccccc54)c3-c3ccccc3)cc2)cc1] | Compound A |
| 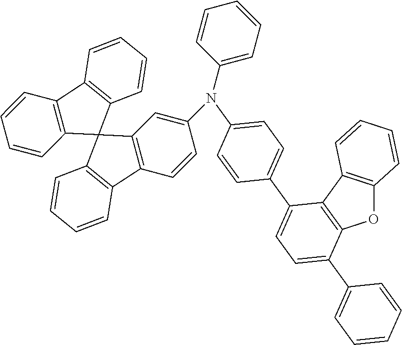 [SMILES: c1ccc(-c2ccc(-c3ccc(N(c4ccccc4)c4ccc5c(c4)C4(c6ccccc6-c6ccccc64)c4ccccc4-5)cc3)c3c2oc2ccccc23)cc1] | Compound B |
| 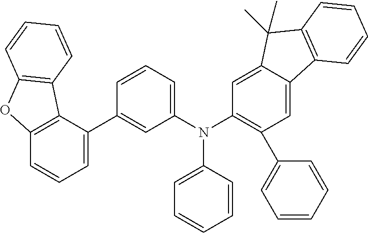 [SMILES: CC1(C)c2ccccc2-c2cc(-c3ccccc3)c(N(c3ccccc3)c3cccc(-c4cccc5oc6ccccc6c45)c3)cc21] | Compound C |
| 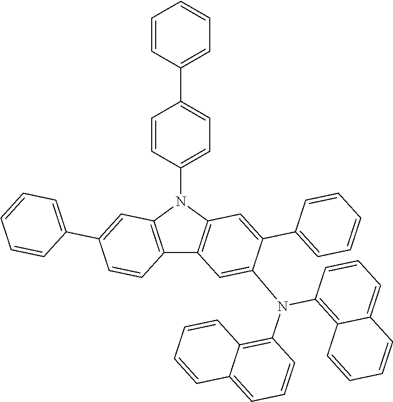 [SMILES: c1ccc(-c2ccc(-n3c4cc(-c5ccccc5)ccc4c4cc(N(c5cccc6ccccc56)c5cccc6ccccc56)c(-c5ccccc5)cc43)cc2)cc1] | Compound D |

The devices prepared in the Examples and the Comparative Examples were tested for performance, wherein the IVL (operating voltage, current efficiency, and chromaticity coordinates) data was tested at a current density of 10 mA/cm² and the T95 lifetime was tested at a current density of 20 mA/cm², and the results are shown in Table 11.

TABLE 11

| | Compound | | | | |
|---|---|---|---|---|---|
| | in second | | | | |
| | hole | Operating | Current | Chromaticity | |
| Example | transport | voltage | efficiency | coordinate | lifetime |
| number | layer | (V) | (Cd/A) | CIEx, CIEy | T95 (h) |
| Example 15 | B-1-2 | 3.90 | 57.65 | 0.680, 0.321 | 485 |
| Example 16 | B-1-5 | 3.82 | 57.87 | 0.679, 0.320 | 487 |
| Example 17 | B-1-6 | 3.87 | 59.16 | 0.680, 0.321 | 489 |
| Example 18 | B-1-19 | 3.80 | 58.66 | 0.679, 0.321 | 483 |
| Example 19 | B-1-20 | 3.82 | 58.79 | 0.679, 0.321 | 488 |
| Example 20 | B-1-29 | 3.83 | 57.74 | 0.679, 0.320 | 481 |
| Example 21 | B-1-36 | 3.85 | 58.15 | 0.680, 0.321 | 486 |
| Example 22 | B-1-37 | 3.92 | 58.32 | 0.679, 0.322 | 476 |
| Example 23 | B-1-43 | 3.86 | 58.44 | 0.679, 0.321 | 482 |
| Example 24 | C-1-1 | 3.85 | 58.02 | 0.680, 0.321 | 480 |
| Example 25 | C-1-7 | 3.93 | 58.59 | 0.680, 0.320 | 486 |
| Example 26 | C-1-10 | 3.88 | 59.06 | 0.680, 0.320 | 478 |
| Example 27 | C-1-14 | 3.94 | 57.97 | 0.679, 0.322 | 484 |
| Example 28 | C-1-18 | 3.91 | 58.05 | 0.679, 0.320 | 473 |
| Example 29 | C-1-19 | 3.89 | 58.91 | 0.681, 0.322 | 477 |
| Example 30 | C-1-23 | 3.92 | 57.75 | 0.679, 0.321 | 490 |
| Example 31 | C-1-24 | 3.84 | 58.72 | 0.680, 0.321 | 475 |
| Example 32 | C-1-32 | 3.80 | 58.64 | 0.679, 0.321 | 479 |
| Example 33 | C-1-35 | 3.97 | 58.83 | 0.680, 0.321 | 474 |
| Example 34 | D-1-6 | 3.93 | 57.82 | 0.680, 0.321 | 467 |
| Example 35 | D-1-27 | 3.97 | 57.67 | 0.681, 0.320 | 469 |
| Comparative | Compound A | 4.09 | 52.11 | 0.679, 0.320 | 420 |
| example 1 | | | | | |
| Comparative | Compound B | 4.14 | 51.19 | 0.679, 0.321 | 415 |
| example 2 | | | | | |
| Comparative | Compound C | 4.27 | 50.08 | 0.680, 0.321 | 408 |
| example 3 | | | | | |
| Comparative | Compound D | 4.20 | 51.04 | 0.680, 0.321 | 411 |
| example 4 | | | | | |

According to the results in Table 11, it can be seen that Examples 15 to 35 employing the organic compounds of the present application as the second hole transport material in the red organic electroluminescent devices, show at least 10.63% increase in the current efficiency and at least 11.2% increase in the lifetime as compared to Comparative Examples 1 to 4 of the device corresponding to the compounds which have been known in the prior art.

Thus, the compounds of the present application have the characteristics of improving luminescence efficiency.

The preferred embodiments of the present application are described in detail above in conjunction with the accompanying drawings. However, the present application is not limited to the specific details of the above embodiments. Within the scope of the technical concept of the present application, various simple modifications can be made to the technical solutions of the present application, and these simple modifications all fall within the scope of protection of the present application.

1. An organic compound having a structure shown by formula I: 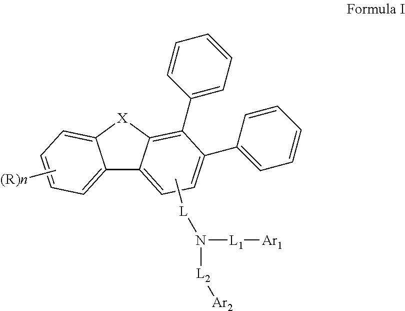
wherein, X is selected from O, S, or N(R₃);
R₃ is selected from an alkyl having 1 to 5 carbon atoms, a deuterated alkyl having 1 to 5 carbon atoms, a haloalkyl having 1 to 5 carbon atoms, an aryl having 6 to 20 carbon atoms, a deuterated aryl having 6 to 20 carbon atoms, or a heteroaryl having 3 to 20 carbon atoms;
L, L₁, and L₂ are the same or different, and are each independently selected from a single bond, a substituted or unsubstituted arylene having 6 to 30 carbon atoms, or a substituted or unsubstituted heteroarylene having 3 to 30 carbon atoms;
Ar₁ and Ar₂ are the same or different, and are each independently selected from a substituted or unsubstituted aryl having 6 to 30 carbon atoms, or a substituted or unsubstituted heteroaryl having 3 to 30 carbon atoms;
the substituent(s) of L, L₁, L₂, Ar₁, and Ar₂ are the same or different, and are each independently selected from a deuterium, a halogen, a cyano, an alkyl having 1 to 10 carbon atoms, a deuterated alkyl having 1 to 10 carbon atoms, a trialkylsilyl having 3 to 12 carbon atoms, a haloalkyl having 1 to 10 carbon atoms, an aryl having 6 to 20 carbon atoms, or a heteroaryl having 3 to 20 carbon atoms;
each R is independently selected from a deuterium, a halogen, a cyano, an alkyl having 1 to 10 carbon atoms, a haloalkyl having 1 to 10 carbon atoms, a deuterated alkyl having 1 to 10 carbon atoms, a trialkylsilyl having 3 to 12 carbon atoms, an aryl having 6 to 20 carbon atoms, or a heteroaryl having 3 to 20 carbon atoms; and
n represents the number of R and n is selected from 0, 1, 2, 3 or 4; when n is greater than 1, any two Rs are the same or different.

2. The organic compound according to claim 1, wherein, each R is independently selected from a deuterium, a fluorine, a cyano, a methyl, an ethyl, an isopropyl, a tert-butyl, a trifluoromethyl, a trimethylsilyl, a phenyl, a naphthyl, a biphenyl, a dibenzofuranyl, a dibenzothienyl, or a carbazolyl.

3. The organic compound according to claim 1, wherein, R₃ is selected from a methyl, an ethyl, an isopropyl, a tert-butyl, a trideuteromethyl, a trifluoromethyl, a phenyl, a deuterophenyl, a naphthyl, a biphenyl, a dibenzofuranyl, a dibenzothienyl, or a carbazolyl.

4. The organic compound according to claim 1, wherein, L, L₁, and L₂ are each independently selected from a single bond, a substituted or unsubstituted arylene having 6 to 18 carbon atoms, or a substituted or unsubstituted heteroarylene having 12 to 18 carbon atoms;
the substituent(s) of L, L₁, and L₂ are each independently selected from a deuterium, a fluorine, a cyano, a trifluoromethyl, a trimethylsilyl, an alkyl having 1 to 5 carbon atoms, or a phenyl.

5. The organic compound according to claim 1, wherein, L, L₁, and L₂ are each independently selected from a single bond, a substituted or unsubstituted phenylene, a substituted or unsubstituted naphthylene, a substituted or unsubstituted biphenylene, a substituted or unsubstituted dibenzofuranylene, a substituted or unsubstituted dibenzothienylene, or a substituted or unsubstituted carbazolylene;
the substituent(s) of L, L₁, and L₂ are each independently selected from a deuterium, a fluorine, a cyano, a trifluoromethyl, a trimethylsilyl, a methyl, an ethyl, an isopropyl, a tert-butyl, or a phenyl.

6. The organic compound according to claim 1, wherein Ar₁ and Ar₂ are each independently selected from a substituted or unsubstituted aryl having 6 to 25 carbon atoms, or a substituted or unsubstituted heteroaryl having 12 to 18 carbon atoms;
the substituent(s) of Ar₁ and Ar₂ are each independently selected from a deuterium, a fluorine, a cyano, a trialkylsilyl having 3 to 6 carbon atoms, a haloalkyl having 1 to 5 carbon atoms, a deuterated alkyl having 1 to 5 carbon atoms, an alkyl having 1 to 5 carbon atoms, an aryl having 6 to 12 carbon atoms, or a heteroaryl having 5 to 12 carbon atoms.

7. The organic compound according to claim 1, wherein, Ar₁ and Ar₂ are each independently selected from a substituted or unsubstituted phenyl, a substituted or unsubstituted naphthyl, a substituted or unsubstituted biphenyl, a substituted or unsubstituted terphenyl, a substituted or unsubstituted fluorenyl, a substituted or unsubstituted 9,9-spirobifluorenyl, a substituted or unsubstituted triphenylene, a substituted or unsubstituted phenanthryl, a substituted or unsubstituted cyclopentanespirofluorene, a substituted or unsubstituted cyclohexanespirofluorene, a substituted or unsubstituted dibenzofuranyl, a substituted or unsubstituted dibenzothienyl, or a substituted or unsubstituted carbazolyl;
the substituent(s) of Ar₁ and Ar₂ are each independently selected from a deuterium, a fluorine, a cyano, trimethylsilyl, a trifluoromethyl, a methyl, an ethyl, an isopropyl, a tert-butyl, a trideuteromethyl, a phenyl, a naphthyl, a biphenyl, a dibenzofuranyl, a dibenzothienyl, or a carbazolyl.

8. The organic compound according to claim 1, wherein, Ar₁ and Ar₂ are each independently selected from the group consisting of the following groups:

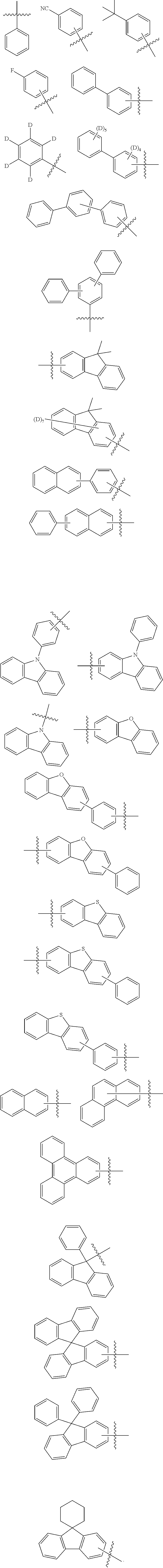

9. The organic compound according to claim 1, wherein, 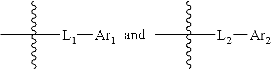
are each independently selected from the group consisting of the following groups:

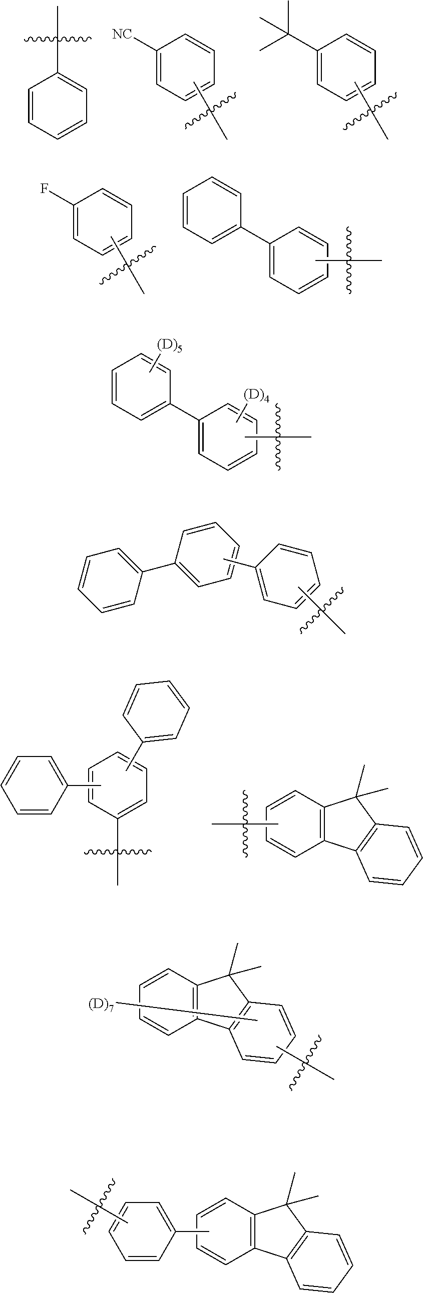

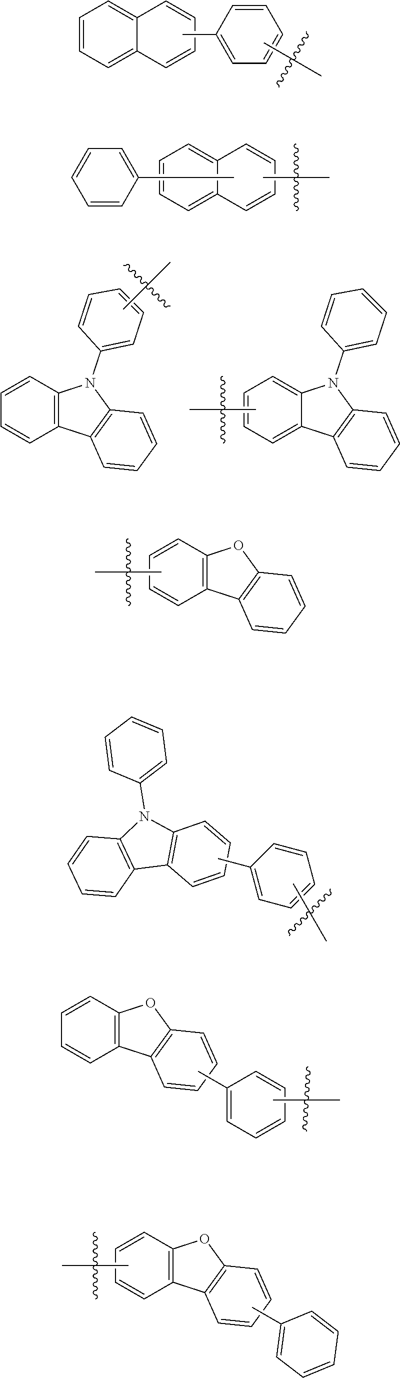

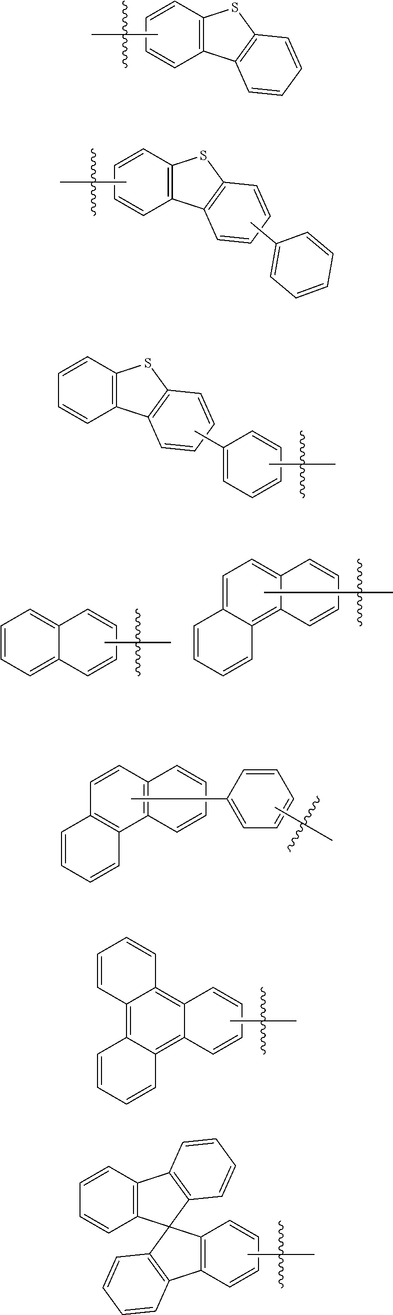

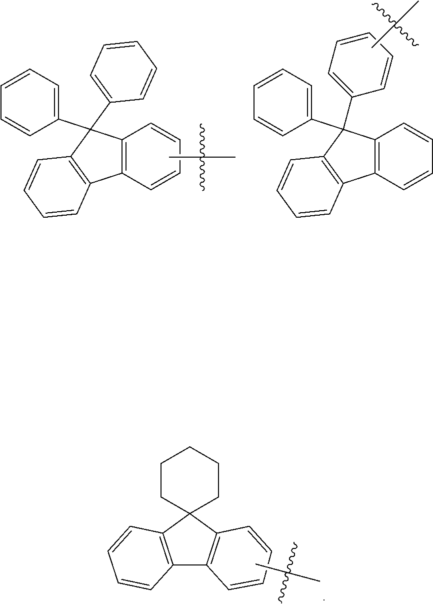

10. The organic compound according to claim 1, wherein the organic compound is selected from the group consisting of the following compounds:

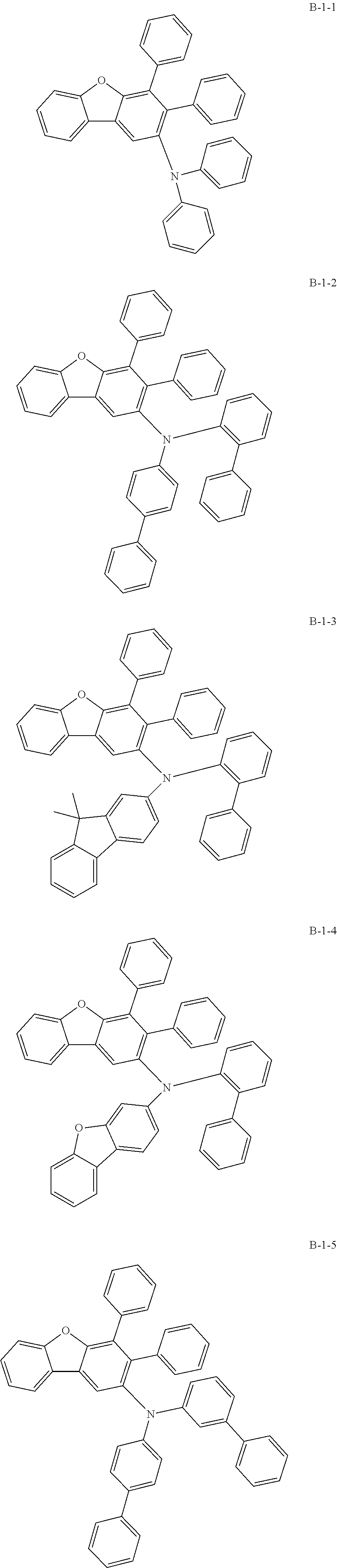 [SMILES: c1ccc(-c2c(N(c3ccccc3)c3ccccc3)cc3c(oc4ccccc43)c2-c2ccccc2)cc1 ; c1ccc(-c2ccc(N(c3ccccc3-c3ccccc3)c3cc4c(oc5ccccc54)c(-c4ccccc4)c3-c3ccccc3)cc2)cc1 ; CC1(C)c2ccccc2-c2ccc(N(c3ccccc3-c3ccccc3)c3cc4c(oc5ccccc54)c(-c4ccccc4)c3-c3ccccc3)cc21 ; c1ccc(-c2ccccc2N(c2ccc3c(c2)oc2ccccc23)c2cc3c(oc4ccccc43)c(-c3ccccc3)c2-c2ccccc2)cc1 ; c1ccc(-c2ccc(N(c3cccc(-c4ccccc4)c3)c3cc4c(oc5ccccc54)c(-c4ccccc4)c3-c3ccccc3)cc2)cc1]

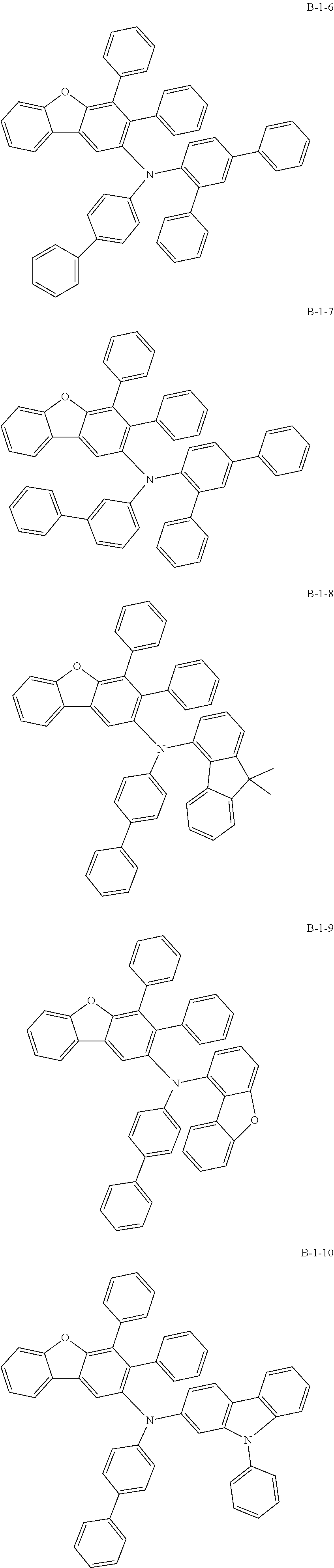 [SMILES: c1ccc(-c2ccc(N(c3ccc(-c4ccccc4)cc3-c3ccccc3)c3cc4c(oc5ccccc54)c(-c4ccccc4)c3-c3ccccc3)cc2)cc1 ; c1ccc(-c2cccc(N(c3ccc(-c4ccccc4)cc3-c3ccccc3)c3cc4c(oc5ccccc54)c(-c4ccccc4)c3-c3ccccc3)c2)cc1 ; CC1(C)c2ccccc2-c2c(N(c3ccc(-c4ccccc4)cc3)c3cc4c(oc5ccccc54)c(-c4ccccc4)c3-c3ccccc3)cccc21 ; c1ccc(-c2ccc(N(c3cc4c(oc5ccccc54)c(-c4ccccc4)c3-c3ccccc3)c3cccc4oc5ccccc5c34)cc2)cc1 ; c1ccc(-c2ccc(N(c3ccc4c5ccccc5n(-c5ccccc5)c4c3)c3cc4c(oc5ccccc54)c(-c4ccccc4)c3-c3ccccc3)cc2)cc1]

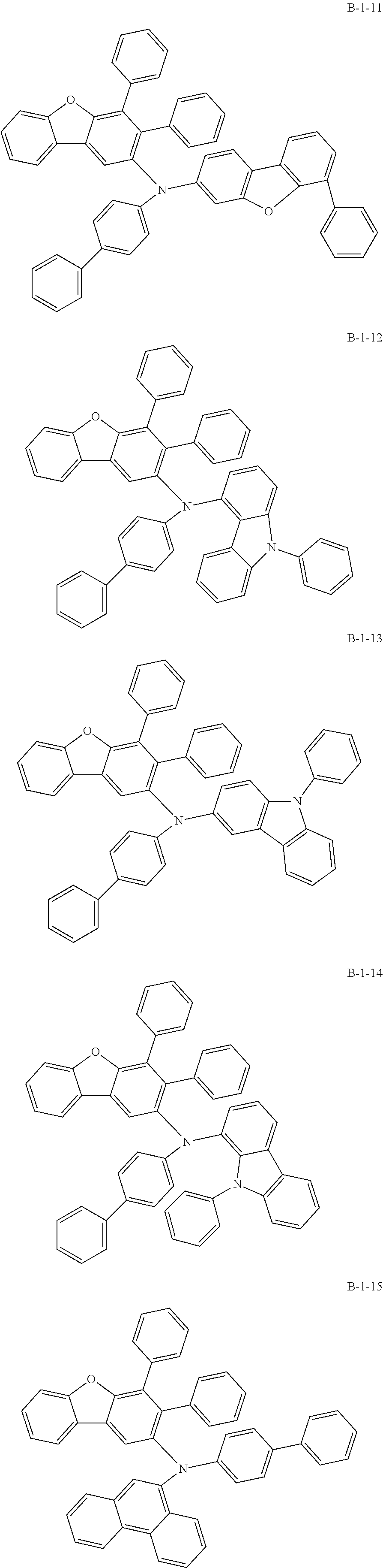 [SMILES: c1ccc(-c2ccc(N(c3ccc4c(c3)oc3c(-c5ccccc5)cccc34)c3cc4c(oc5ccccc54)c(-c4ccccc4)c3-c3ccccc3)cc2)cc1 ; c1ccc(-c2ccc(N(c3cc4c(oc5ccccc54)c(-c4ccccc4)c3-c3ccccc3)c3cccc4c3c3ccccc3n4-c3ccccc3)cc2)cc1 ; c1ccc(-c2ccc(N(c3ccc4c(c3)c3ccccc3n4-c3ccccc3)c3cc4c(oc5ccccc54)c(-c4ccccc4)c3-c3ccccc3)cc2)cc1 ; c1ccc(-c2ccc(N(c3cc4c(oc5ccccc54)c(-c4ccccc4)c3-c3ccccc3)c3cccc4c5ccccc5n(-c5ccccc5)c34)cc2)cc1 ; c1ccc(-c2ccc(N(c3cc4c(oc5ccccc54)c(-c4ccccc4)c3-c3ccccc3)c3cc4ccccc4c4ccccc34)cc2)cc1]

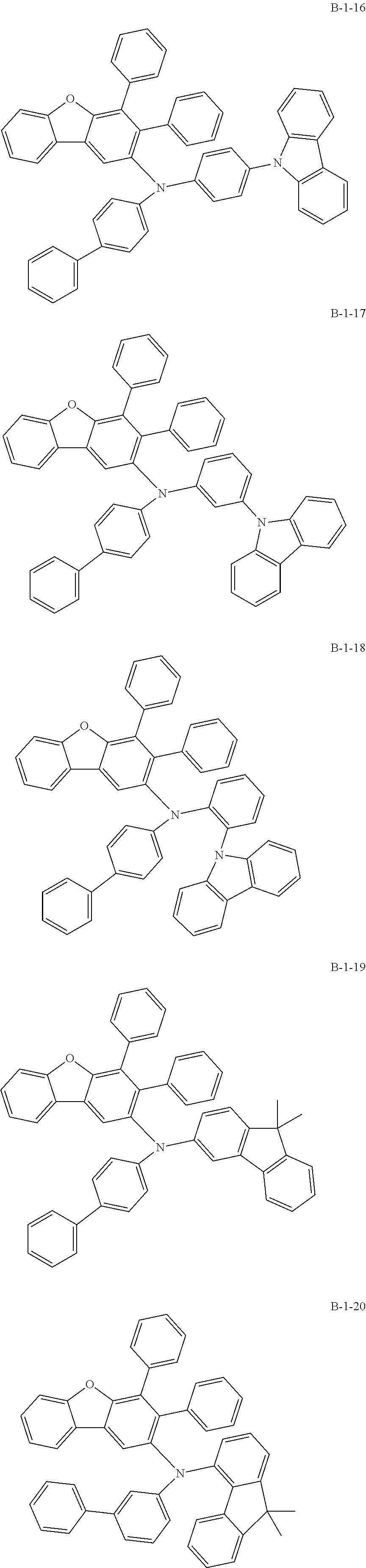 [SMILES: c1ccc(-c2ccc(N(c3ccc(-n4c5ccccc5c5ccccc54)cc3)c3cc4c(oc5ccccc54)c(-c4ccccc4)c3-c3ccccc3)cc2)cc1 ; c1ccc(-c2ccc(N(c3cccc(-n4c5ccccc5c5ccccc54)c3)c3cc4c(oc5ccccc54)c(-c4ccccc4)c3-c3ccccc3)cc2)cc1 ; c1ccc(-c2ccc(N(c3ccccc3-n3c4ccccc4c4ccccc43)c3cc4c(oc5ccccc54)c(-c4ccccc4)c3-c3ccccc3)cc2)cc1 ; CC1(C)c2ccccc2-c2cc(N(c3ccc(-c4ccccc4)cc3)c3cc4c(oc5ccccc54)c(-c4ccccc4)c3-c3ccccc3)ccc21 ; CC1(C)c2ccccc2-c2c(N(c3cccc(-c4ccccc4)c3)c3cc4c(oc5ccccc54)c(-c4ccccc4)c3-c3ccccc3)cccc21]

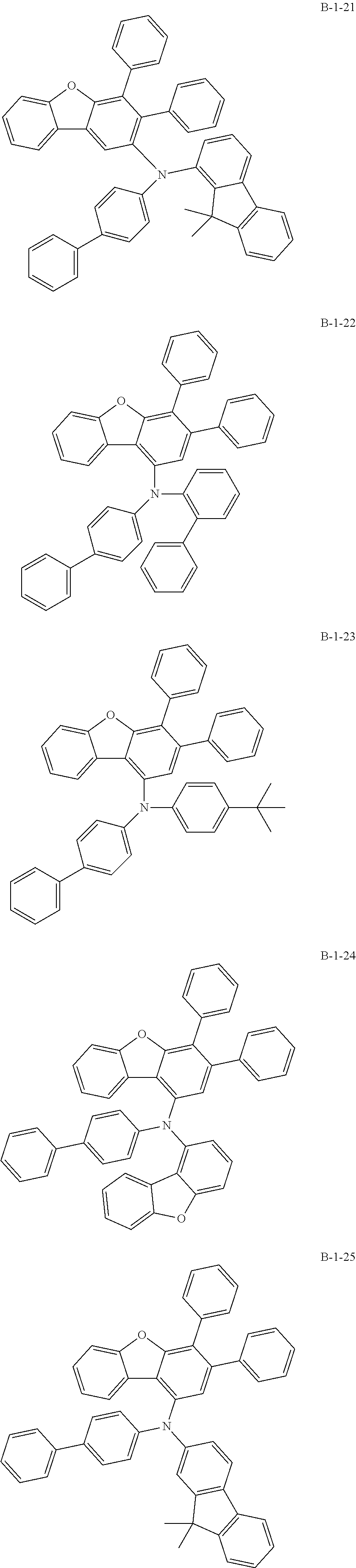 [SMILES: CC1(C)c2ccccc2-c2cccc(N(c3ccc(-c4ccccc4)cc3)c3cc4c(oc5ccccc54)c(-c4ccccc4)c3-c3ccccc3)c21 ; c1ccc(-c2ccc(N(c3ccccc3-c3ccccc3)c3cc(-c4ccccc4)c(-c4ccccc4)c4oc5ccccc5c34)cc2)cc1 ; CC(C)(C)c1ccc(N(c2ccc(-c3ccccc3)cc2)c2cc(-c3ccccc3)c(-c3ccccc3)c3oc4ccccc4c23)cc1 ; c1ccc(-c2ccc(N(c3cccc4oc5ccccc5c34)c3cc(-c4ccccc4)c(-c4ccccc4)c4oc5ccccc5c34)cc2)cc1 ; CC1(C)c2ccccc2-c2ccc(N(c3ccc(-c4ccccc4)cc3)c3cc(-c4ccccc4)c(-c4ccccc4)c4oc5ccccc5c34)cc21]

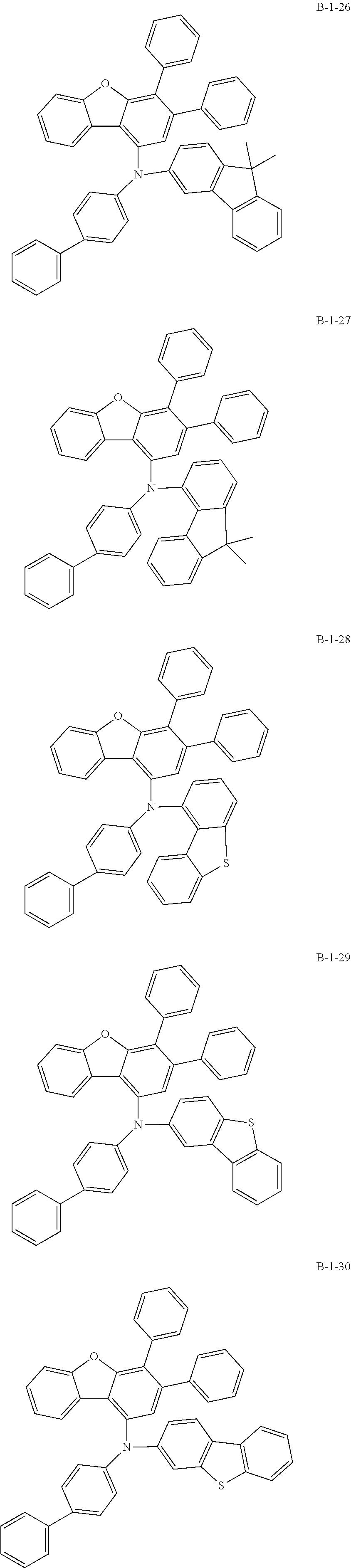 [SMILES: CC1(C)c2ccccc2-c2cc(N(c3ccc(-c4ccccc4)cc3)c3cc(-c4ccccc4)c(-c4ccccc4)c4oc5ccccc5c34)ccc21 ; CC1(C)c2ccccc2-c2c(N(c3ccc(-c4ccccc4)cc3)c3cc(-c4ccccc4)c(-c4ccccc4)c4oc5ccccc5c34)cccc21 ; c1ccc(-c2ccc(N(c3cccc4sc5ccccc5c34)c3cc(-c4ccccc4)c(-c4ccccc4)c4oc5ccccc5c34)cc2)cc1 ; c1ccc(-c2ccc(N(c3ccc4sc5ccccc5c4c3)c3cc(-c4ccccc4)c(-c4ccccc4)c4oc5ccccc5c34)cc2)cc1 ; c1ccc(-c2ccc(N(c3ccc4c(c3)sc3ccccc34)c3cc(-c4ccccc4)c(-c4ccccc4)c4oc5ccccc5c34)cc2)cc1]

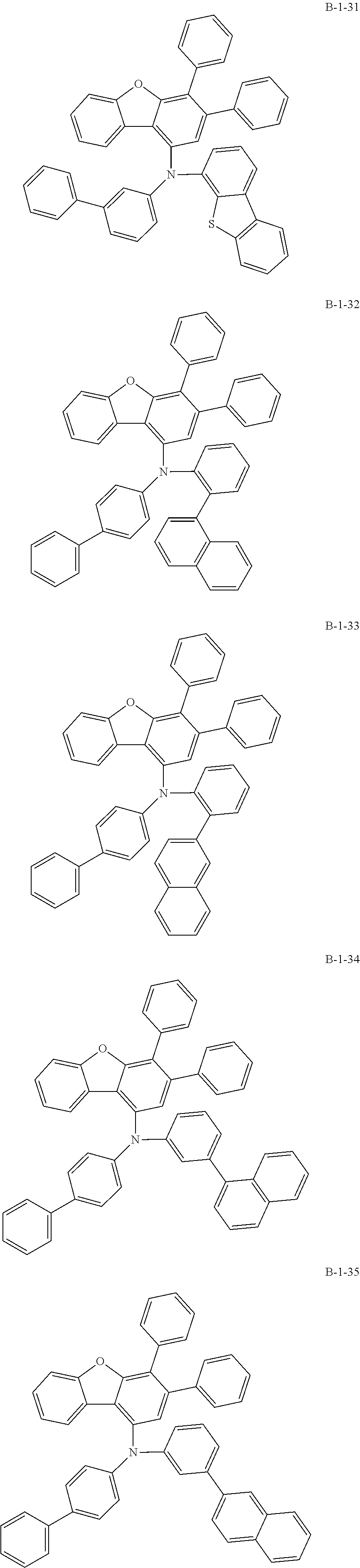 [SMILES: c1ccc(-c2cccc(N(c3cccc4c3sc3ccccc34)c3cc(-c4ccccc4)c(-c4ccccc4)c4oc5ccccc5c34)c2)cc1 ; c1ccc(-c2ccc(N(c3ccccc3-c3cccc4ccccc34)c3cc(-c4ccccc4)c(-c4ccccc4)c4oc5ccccc5c34)cc2)cc1 ; c1ccc(-c2ccc(N(c3ccccc3-c3ccc4ccccc4c3)c3cc(-c4ccccc4)c(-c4ccccc4)c4oc5ccccc5c34)cc2)cc1 ; c1ccc(-c2ccc(N(c3cccc(-c4cccc5ccccc45)c3)c3cc(-c4ccccc4)c(-c4ccccc4)c4oc5ccccc5c34)cc2)cc1 ; c1ccc(-c2ccc(N(c3cccc(-c4ccc5ccccc5c4)c3)c3cc(-c4ccccc4)c(-c4ccccc4)c4oc5ccccc5c34)cc2)cc1]

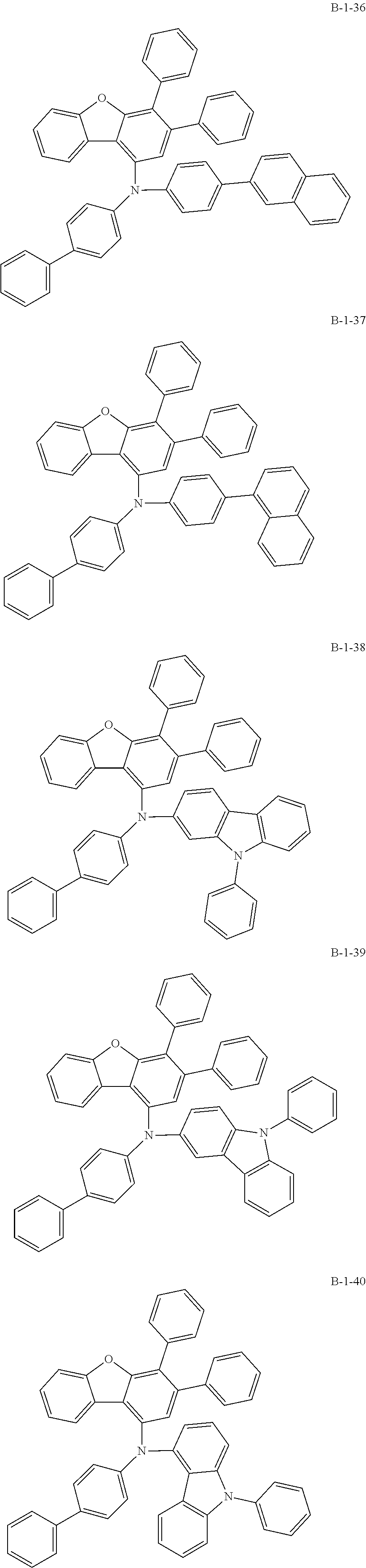 [SMILES: c1ccc(-c2ccc(N(c3ccc(-c4ccc5ccccc5c4)cc3)c3cc(-c4ccccc4)c(-c4ccccc4)c4oc5ccccc5c34)cc2)cc1 ; c1ccc(-c2ccc(N(c3ccc(-c4cccc5ccccc45)cc3)c3cc(-c4ccccc4)c(-c4ccccc4)c4oc5ccccc5c34)cc2)cc1 ; c1ccc(-c2ccc(N(c3ccc4c5ccccc5n(-c5ccccc5)c4c3)c3cc(-c4ccccc4)c(-c4ccccc4)c4oc5ccccc5c34)cc2)cc1 ; c1ccc(-c2ccc(N(c3ccc4c(c3)c3ccccc3n4-c3ccccc3)c3cc(-c4ccccc4)c(-c4ccccc4)c4oc5ccccc5c34)cc2)cc1 ; c1ccc(-c2ccc(N(c3cc(-c4ccccc4)c(-c4ccccc4)c4oc5ccccc5c34)c3cccc4c3c3ccccc3n4-c3ccccc3)cc2)cc1]

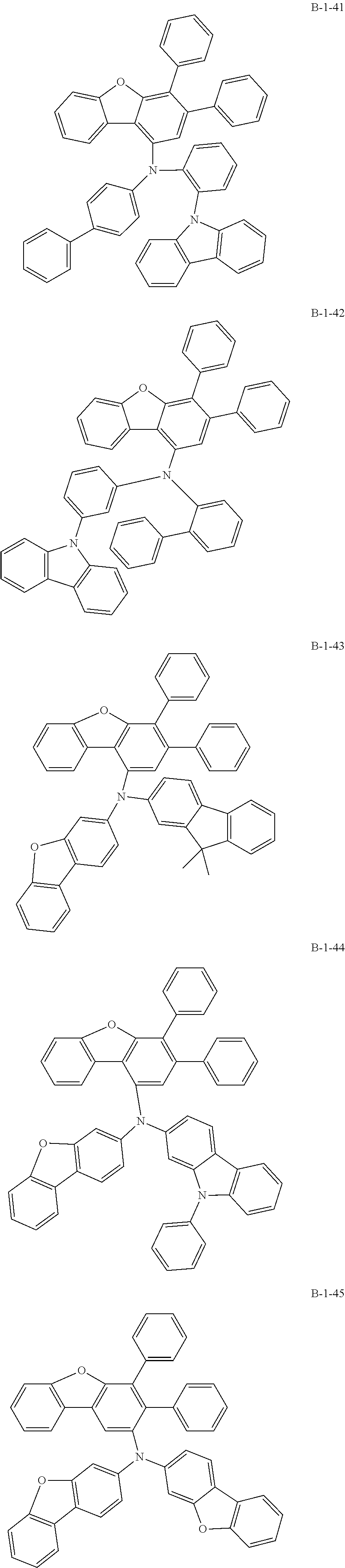 [SMILES: c1ccc(-c2ccc(N(c3ccccc3-n3c4ccccc4c4ccccc43)c3cc(-c4ccccc4)c(-c4ccccc4)c4oc5ccccc5c34)cc2)cc1 ; c1ccc(-c2ccccc2N(c2cccc(-n3c4ccccc4c4ccccc43)c2)c2cc(-c3ccccc3)c(-c3ccccc3)c3oc4ccccc4c23)cc1 ; CC1(C)c2ccccc2-c2ccc(N(c3ccc4c(c3)oc3ccccc34)c3cc(-c4ccccc4)c(-c4ccccc4)c4oc5ccccc5c34)cc21 ; c1ccc(-c2cc(N(c3ccc4c(c3)oc3ccccc34)c3ccc4c5ccccc5n(-c5ccccc5)c4c3)c3c(oc4ccccc43)c2-c2ccccc2)cc1 ; c1ccc(-c2c(N(c3ccc4c(c3)oc3ccccc34)c3ccc4c(c3)oc3ccccc34)cc3c(oc4ccccc43)c2-c2ccccc2)cc1]

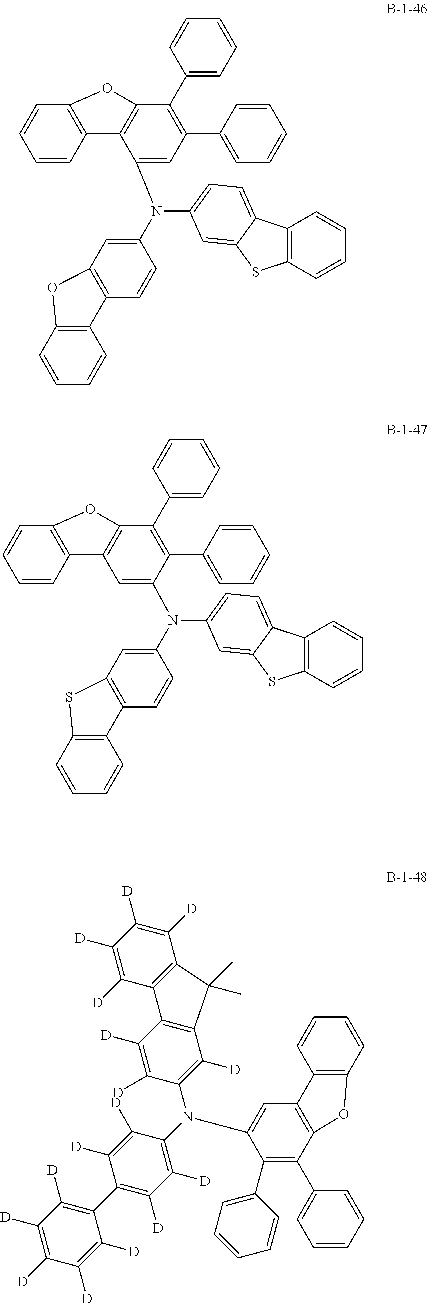 [SMILES: c1ccc(-c2cc(N(c3ccc4c(c3)oc3ccccc34)c3ccc4c(c3)sc3ccccc34)c3c(oc4ccccc43)c2-c2ccccc2)cc1 ; c1ccc(-c2c(N(c3ccc4c(c3)sc3ccccc34)c3ccc4c(c3)sc3ccccc34)cc3c(oc4ccccc43)c2-c2ccccc2)cc1 ; [2H]c1c([2H])c([2H])c(-c2c([2H])c([2H])c(N(c3cc4c(oc5ccccc54)c(-c4ccccc4)c3-c3ccccc3)c3c([2H])c([2H])c4c(c3[2H])C(C)(C)c3c([2H])c([2H])c([2H])c([2H])c3-4)c([2H])c2[2H])c([2H])c1[2H]]

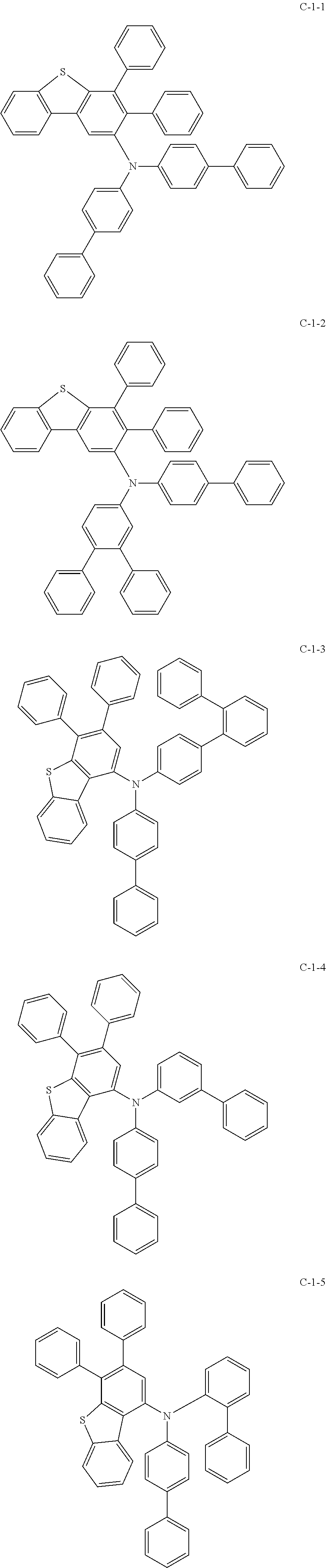 [SMILES: c1ccc(-c2ccc(N(c3ccc(-c4ccccc4)cc3)c3cc4c(sc5ccccc54)c(-c4ccccc4)c3-c3ccccc3)cc2)cc1 ; c1ccc(-c2ccc(N(c3ccc(-c4ccccc4)c(-c4ccccc4)c3)c3cc4c(sc5ccccc54)c(-c4ccccc4)c3-c3ccccc3)cc2)cc1 ; c1ccc(-c2ccc(N(c3ccc(-c4ccccc4-c4ccccc4)cc3)c3cc(-c4ccccc4)c(-c4ccccc4)c4sc5ccccc5c34)cc2)cc1 ; c1ccc(-c2ccc(N(c3cccc(-c4ccccc4)c3)c3cc(-c4ccccc4)c(-c4ccccc4)c4sc5ccccc5c34)cc2)cc1 ; c1ccc(-c2ccc(N(c3ccccc3-c3ccccc3)c3cc(-c4ccccc4)c(-c4ccccc4)c4sc5ccccc5c34)cc2)cc1]

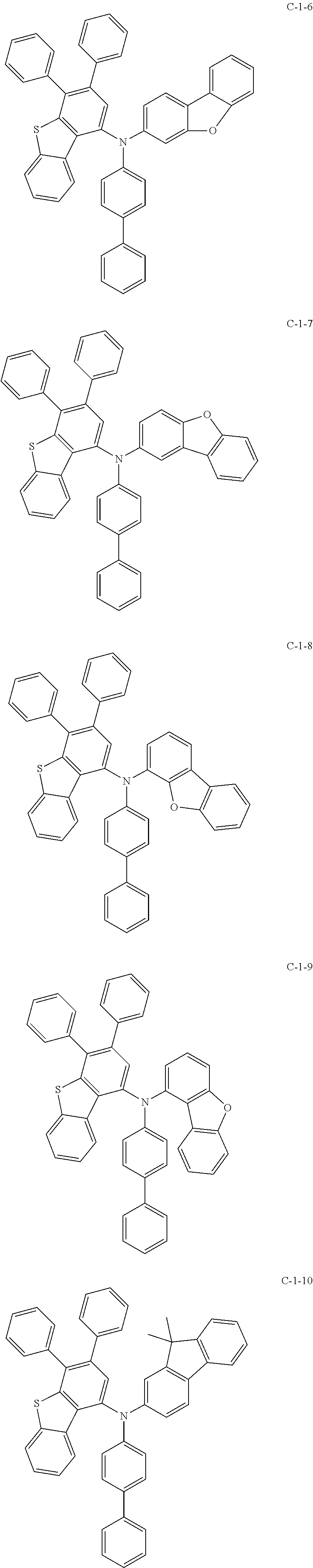 [SMILES: c1ccc(-c2ccc(N(c3ccc4c(c3)oc3ccccc34)c3cc(-c4ccccc4)c(-c4ccccc4)c4sc5ccccc5c34)cc2)cc1 ; c1ccc(-c2ccc(N(c3ccc4oc5ccccc5c4c3)c3cc(-c4ccccc4)c(-c4ccccc4)c4sc5ccccc5c34)cc2)cc1 ; c1ccc(-c2ccc(N(c3cccc4c3oc3ccccc34)c3cc(-c4ccccc4)c(-c4ccccc4)c4sc5ccccc5c34)cc2)cc1 ; c1ccc(-c2ccc(N(c3cccc4oc5ccccc5c34)c3cc(-c4ccccc4)c(-c4ccccc4)c4sc5ccccc5c34)cc2)cc1 ; CC1(C)c2ccccc2-c2ccc(N(c3ccc(-c4ccccc4)cc3)c3cc(-c4ccccc4)c(-c4ccccc4)c4sc5ccccc5c34)cc21]

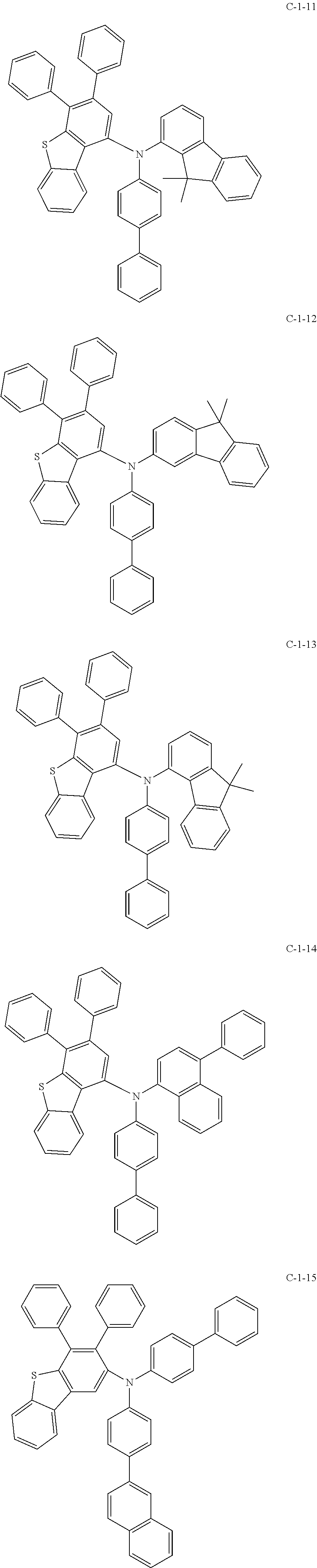 [SMILES: CC1(C)c2ccccc2-c2cccc(N(c3ccc(-c4ccccc4)cc3)c3cc(-c4ccccc4)c(-c4ccccc4)c4sc5ccccc5c34)c21 ; CC1(C)c2ccccc2-c2cc(N(c3ccc(-c4ccccc4)cc3)c3cc(-c4ccccc4)c(-c4ccccc4)c4sc5ccccc5c34)ccc21 ; CC1(C)c2ccccc2-c2c(N(c3ccc(-c4ccccc4)cc3)c3cc(-c4ccccc4)c(-c4ccccc4)c4sc5ccccc5c34)cccc21 ; c1ccc(-c2ccc(N(c3ccc(-c4ccccc4)c4ccccc34)c3cc(-c4ccccc4)c(-c4ccccc4)c4sc5ccccc5c34)cc2)cc1 ; c1ccc(-c2ccc(N(c3ccc(-c4ccc5ccccc5c4)cc3)c3cc4c(sc5ccccc54)c(-c4ccccc4)c3-c3ccccc3)cc2)cc1]

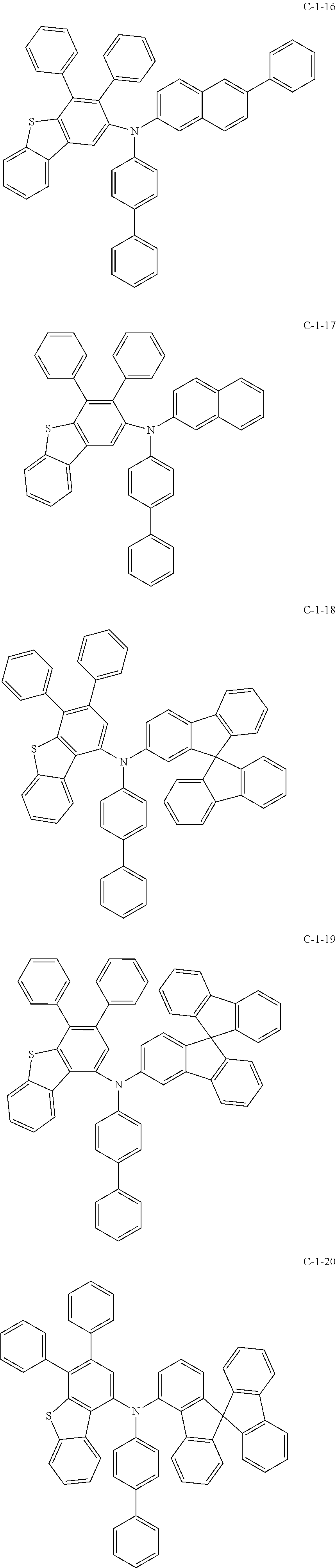 [SMILES: c1ccc(-c2ccc(N(c3ccc4cc(-c5ccccc5)ccc4c3)c3cc4c(sc5ccccc54)c(-c4ccccc4)c3-c3ccccc3)cc2)cc1 ; c1ccc(-c2ccc(N(c3ccc4ccccc4c3)c3cc4c(sc5ccccc54)c(-c4ccccc4)c3-c3ccccc3)cc2)cc1 ; c1ccc(-c2ccc(N(c3ccc4c(c3)C3(c5ccccc5-c5ccccc53)c3ccccc3-4)c3cc(-c4ccccc4)c(-c4ccccc4)c4sc5ccccc5c34)cc2)cc1 ; c1ccc(-c2ccc(N(c3ccc4c(c3)-c3ccccc3C43c4ccccc4-c4ccccc43)c3cc(-c4ccccc4)c(-c4ccccc4)c4sc5ccccc5c34)cc2)cc1 ; c1ccc(-c2ccc(N(c3cccc4c3-c3ccccc3C43c4ccccc4-c4ccccc43)c3cc(-c4ccccc4)c(-c4ccccc4)c4sc5ccccc5c34)cc2)cc1]

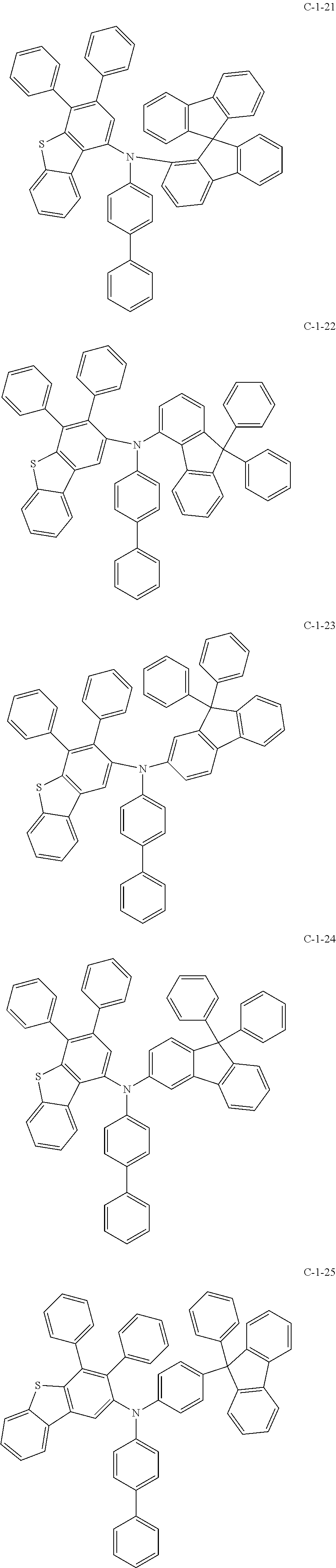

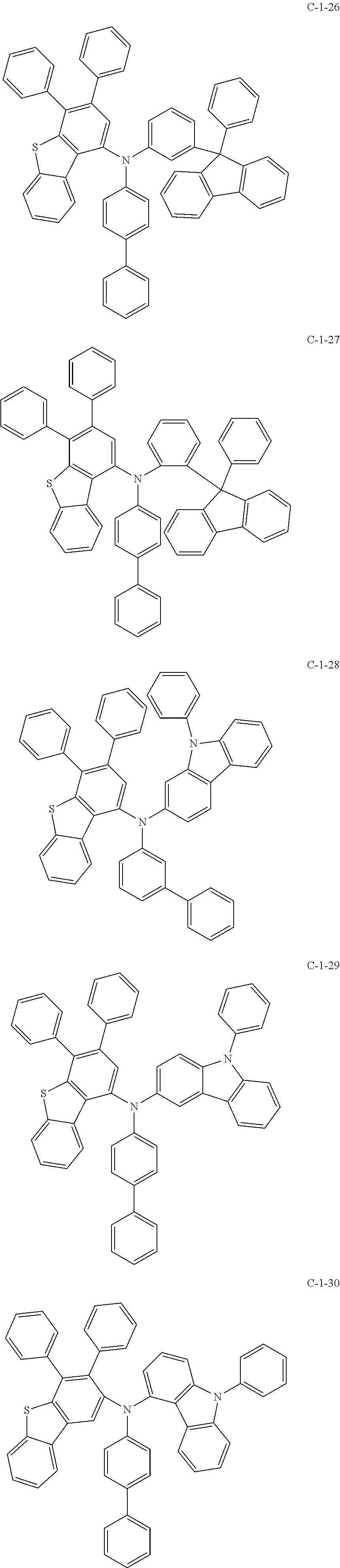

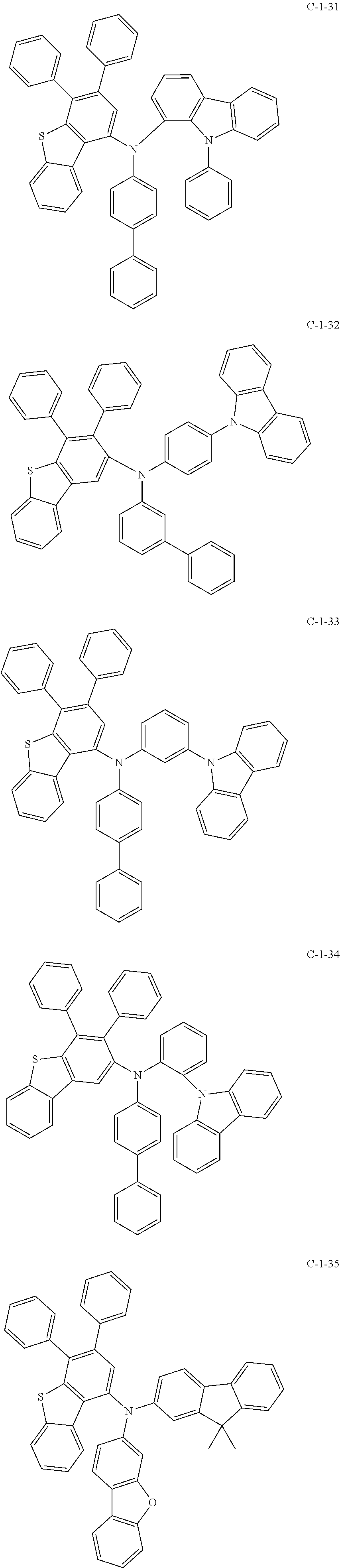 [SMILES: c1ccc(-c2ccc(N(c3cc(-c4ccccc4)c(-c4ccccc4)c4sc5ccccc5c34)c3cccc4c5ccccc5n(-c5ccccc5)c34)cc2)cc1 ; c1ccc(-c2cccc(N(c3ccc(-n4c5ccccc5c5ccccc54)cc3)c3cc4c(sc5ccccc54)c(-c4ccccc4)c3-c3ccccc3)c2)cc1 ; c1ccc(-c2ccc(N(c3cccc(-n4c5ccccc5c5ccccc54)c3)c3cc(-c4ccccc4)c(-c4ccccc4)c4sc5ccccc5c34)cc2)cc1 ; c1ccc(-c2ccc(N(c3ccccc3-n3c4ccccc4c4ccccc43)c3cc4c(sc5ccccc54)c(-c4ccccc4)c3-c3ccccc3)cc2)cc1 ; CC1(C)c2ccccc2-c2ccc(N(c3ccc4c(c3)oc3ccccc34)c3cc(-c4ccccc4)c(-c4ccccc4)c4sc5ccccc5c34)cc21]

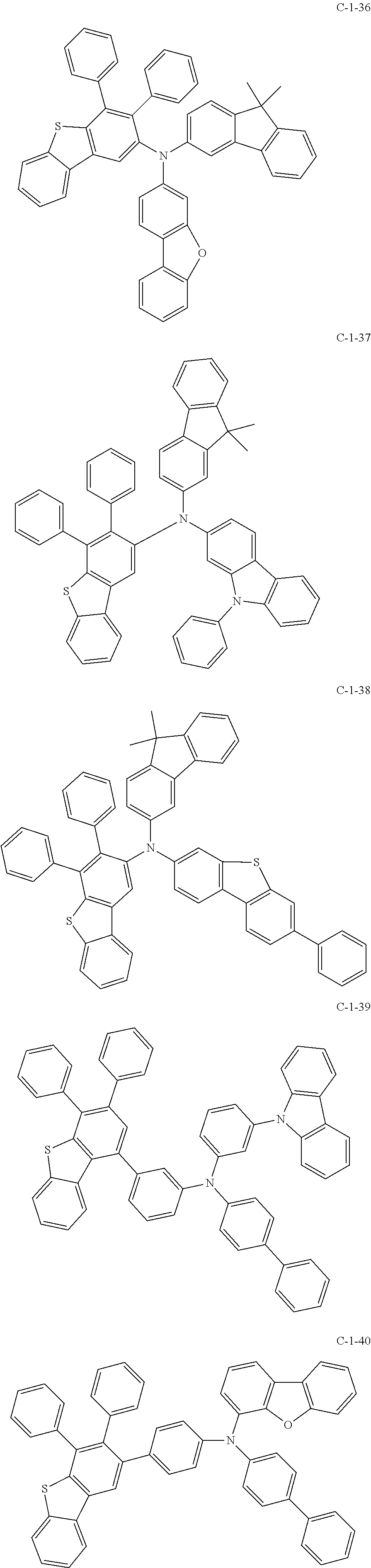

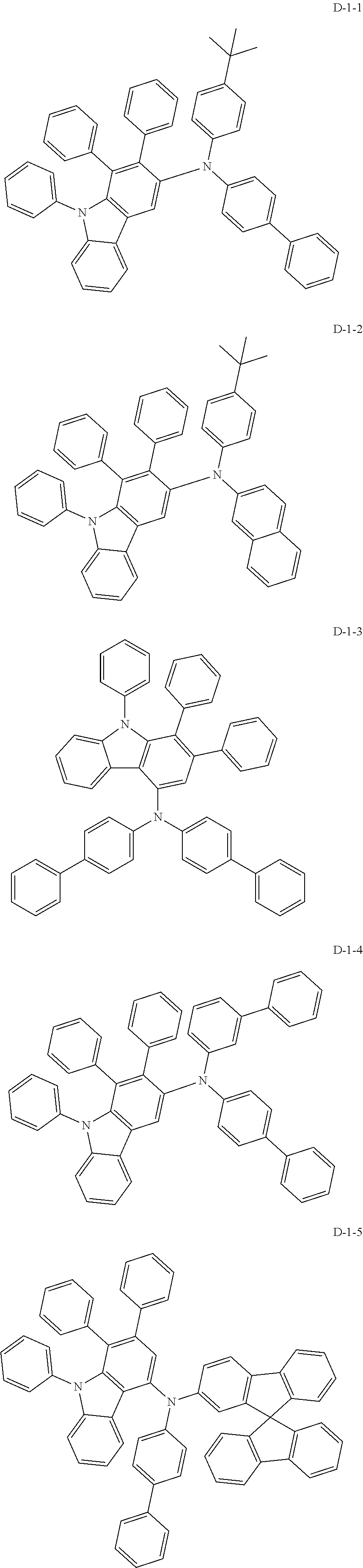

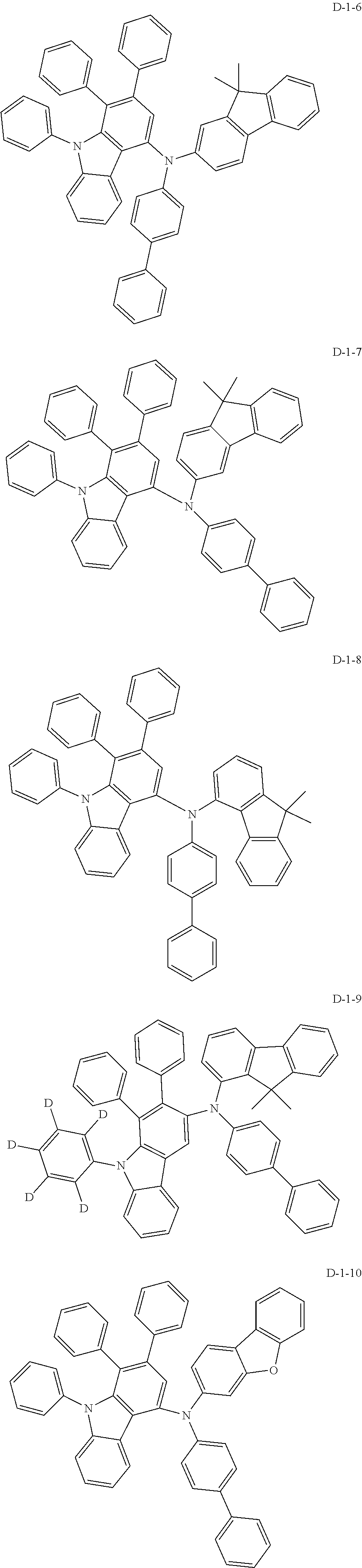

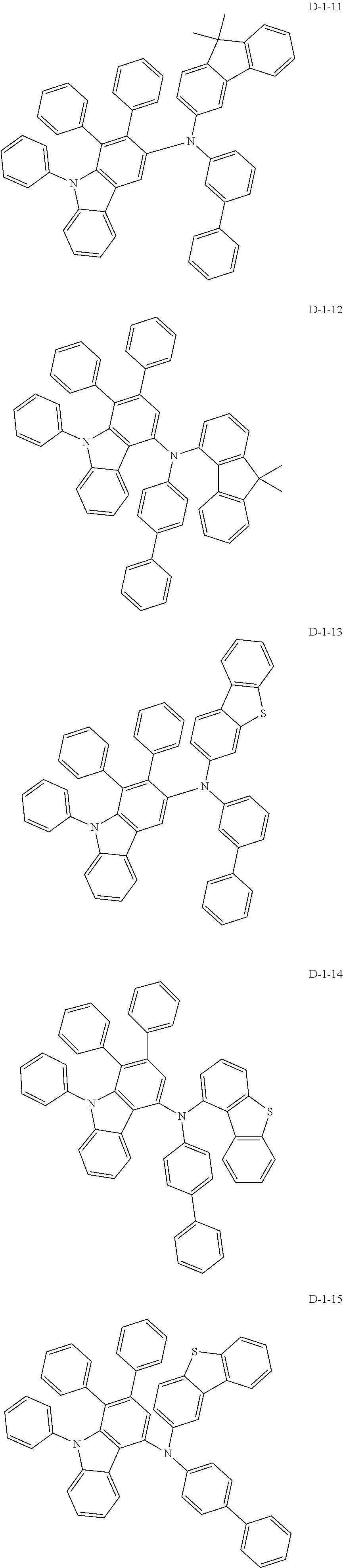 [SMILES: CC1(C)c2ccccc2-c2cc(N(c3cccc(-c4ccccc4)c3)c3cc4c5ccccc5n(-c5ccccc5)c4c(-c4ccccc4)c3-c3ccccc3)ccc21 ; CC1(C)c2ccccc2-c2c(N(c3ccc(-c4ccccc4)cc3)c3cc(-c4ccccc4)c(-c4ccccc4)c4c3c3ccccc3n4-c3ccccc3)cccc21 ; c1ccc(-c2cccc(N(c3ccc4c(c3)sc3ccccc34)c3cc4c5ccccc5n(-c5ccccc5)c4c(-c4ccccc4)c3-c3ccccc3)c2)cc1 ; c1ccc(-c2ccc(N(c3cccc4sc5ccccc5c34)c3cc(-c4ccccc4)c(-c4ccccc4)c4c3c3ccccc3n4-c3ccccc3)cc2)cc1 ; c1ccc(-c2ccc(N(c3ccc4sc5ccccc5c4c3)c3cc(-c4ccccc4)c(-c4ccccc4)c4c3c3ccccc3n4-c3ccccc3)cc2)cc1]

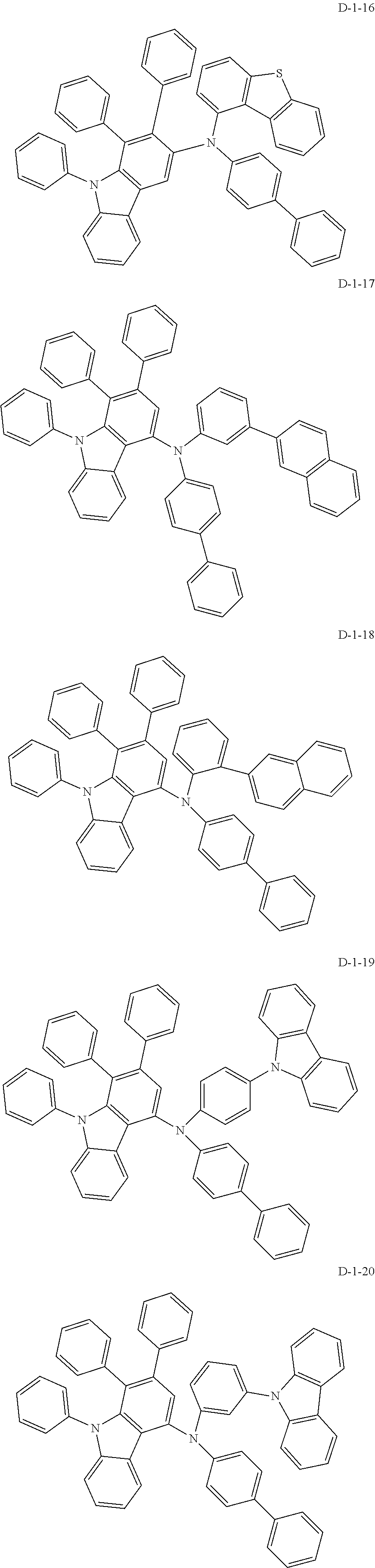

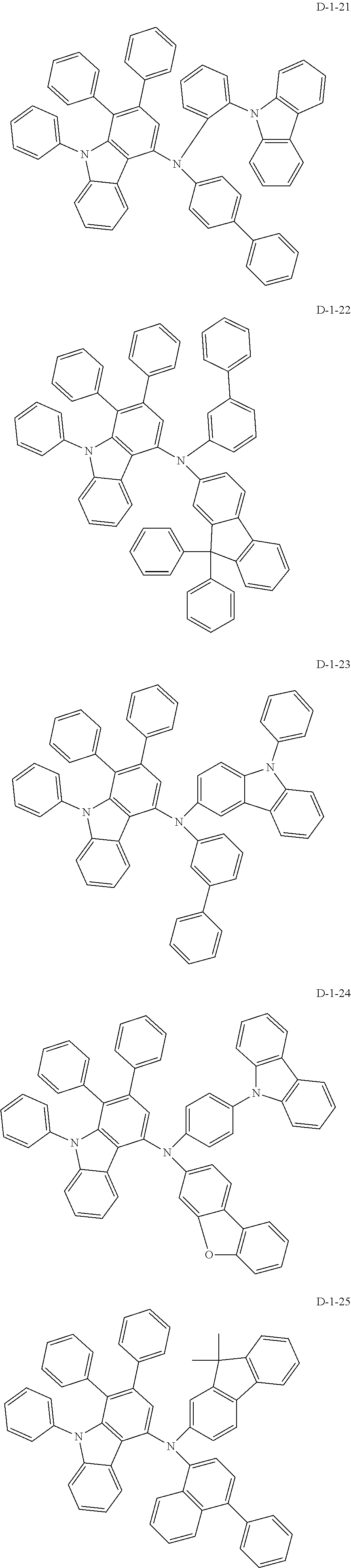

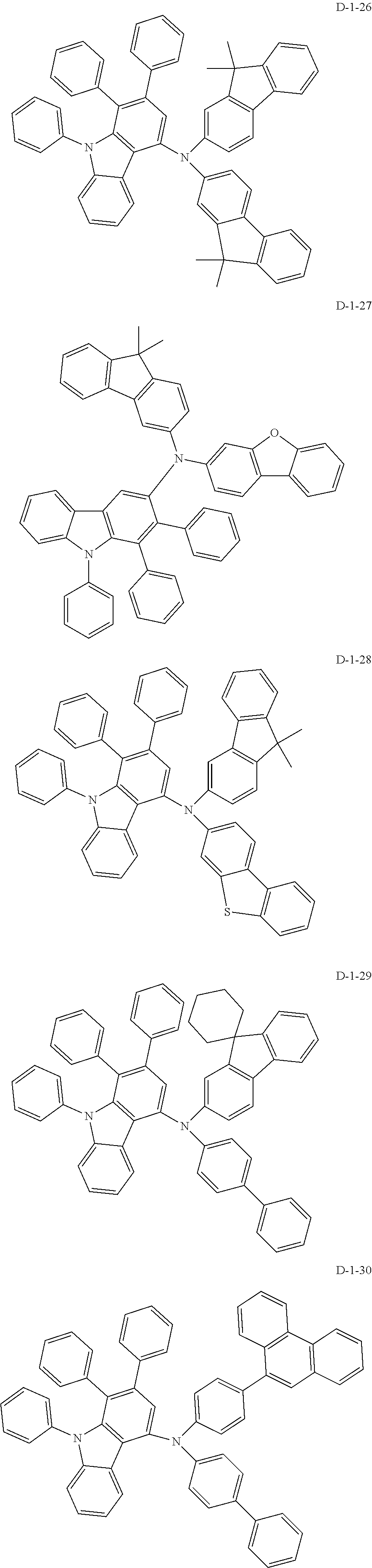

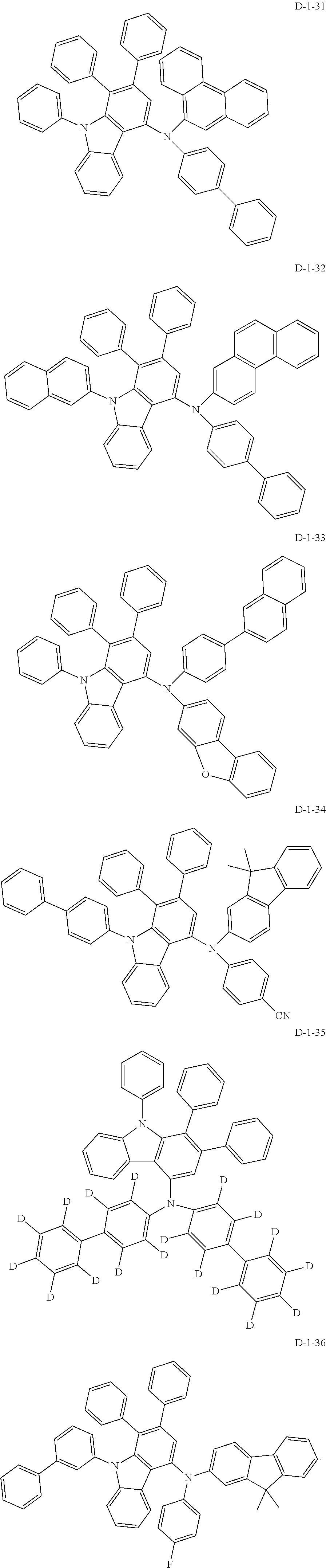

11. An electronic element, comprising an anode and a cathode disposed opposite each other, and a functional layer disposed between the anode and the cathode; wherein the functional layer comprises the organic compound according to claim 1.

12. The electronic element according to claim 11, wherein the functional layer comprises a hole transport layer, comprising the organic compound;
alternatively, the electronic element is an organic electroluminescent device or a photoelectric conversion device;
alternatively, the organic electroluminescent device is a red-light organic electroluminescent device.

13. An electronic apparatus, comprising the electronic element according to claim 11.
